(19) United States
(12) Patent Application Publication
(10) Pub. No.: US 2021/0190798 A1
(43) Pub. Date: Jun. 24, 2021

(54) METHODS OF QUANTIFYING LIF AND USES THEREOF

(71) Applicants: MEDIMMUNE LIMITED, Camebridge (GB); FUNDACIO PRIVADA INSTITUT D'INVESTIGACIO ONCOLOGICA DE VALL HEBRON, Barcelona (ES); FUNDACIO PRIVADA INSTITUCIO CATALANA DE RECERCA ESTUDIS AVANCATS, Barcelona (ES)

(72) Inventors: Joan SEOANE SUAREZ, Barcelona (ES); Judit ANIDO FOLGUEIRA, Barcelona (ES); Peter Edward BAYLISS, Toronto, ON (CA); Patricia Anne GIBLIN, Boston, MA (US); Johan FRANSSON, Toronto, ON (CA); Arif JETHA, Toronto, ON (CA)

(21) Appl. No.: 17/252,476

(22) PCT Filed: Jun. 17, 2019
(86) PCT No.: PCT/IB2019/000756

§ 371 Date: Dec. 15, 2020

(30) Foreign Application Priority Data
Jun. 18, 2018 (EP) 18382433.3

(51) Int. Cl. G01N 33/68; C07K 14/54; C07K 16/24

(52) U.S. Cl. CPC G01N 33/6869; C07K 14/5415; G01N 2333/5415; C07K 2317/24; C07K 2317/92; C07K 16/244

(57) ABSTRACT

Described herein are assays useful for the quantitation of total LIF from biological samples.

### CROSS REFERENCE TO RELATED APPLICATIONS

The present application claims the benefit of European Application Serial Number 18382433.3, filed Jun. 18, 2018, the contents of which is hereby incorporated by reference in its entirety.

### BACKGROUND

Leukemia inhibitory factor (LIF) is a member of the interleukin-6 (IL6) family of cytokines. Based on the known physiological activities of LIF, clinical features of LIF deficiency, and nonclinical studies of LIF expression in healthy tissues and tumors, inhibition of LIF is expected to be well tolerated and to result in anti-tumor efficacy in a range of solid tumors, including but not limited to non-small cell lung cancer (NSCLC), pancreatic cancer, ovarian cancer and glioblastoma multiforme (GBM).

LIF signals by binding to LIFR and then recruiting gp130 to form the ternary complex capable of initiating intracellular JAK-STAT activation. Thus, LIF has binding sites for both LIFR and gp130, providing multiple options for inhibiting LIF signaling. One strategy would be to directly block LIF cytokine binding to LIFR. Alternatively, an additional strategy to inhibit LIF signaling is to block LIF binding to gp130. In this scenario, a mAb that binds an epitope overlapping with the gp130 binding site of LIF prevents downstream signaling by inhibiting recruitment of gp130 to the LIF/LIFR complex, which is necessary for signal transduction.

A typical plasma or serum concentration of LIF in normal healthy individuals is between 0-10 pg/ml. However, since the clearance of antibody/ligand complex is slower than the clearance of free ligand, the levels of total LIF in plasma may increase once the cytokine is bound to an antibody. See e.g., Chakraborty A, Tannenbaum S, Rordorf C, et al. (2012) “Pharmacokinetic and Pharmacodynamic Properties of Canakinumab, a Human Anti-Interleukin-1 β Monoclonal Antibody” Clin Pharmacokinet. 51:e1-e18; Dudai S, Subramanian K, Flandre T, et al. (2015) “Integrated pharmacokinetic, pharmacodynamics and immunogenicity profiling of an anti-CCL21 monoclonal antibody in cynomolgus monkeys” mAbs. 7:829-837. Thus, the accumulation of either the drug-ligand complex or total ligand, and the duration of saturation, can be incorporated into PK-PD modeling for prediction of target engagement and efficacy in the periphery.

### SUMMARY

The present disclosure relates to the use of anti-leukemia inhibitory factor (LIF) antibodies for the detection of total LIF levels in patient samples, such as blood, plasma or serum, following administration of a therapeutic antibody, which binds at or near the gp130-binding site of LIF. In an enzyme linked immunosorbent assay (ELISA) using the sandwich method described herein, LIF can be quantitatively detected by sandwiching it between an immobilized capture antibody and another antibody which is conjugated to a detectable labelling substance (detection antibody), wherein these antibodies bind to non-overlapping epitopes of LIF. In the present disclosure, the capture and detection antibody epitopes of LIF are additionally both non-overlapping with the binding site of the therapeutic mAb h5D8. Thus, three distinct non-overlapping epitopes of LIF have been identified such that three antibodies (capture, detection and therapeutic) can bind LIF simultaneously. An advantage of this format is that LIF can be detected whether or not it is bound by the therapeutic antibody, and thus quantifies the “total LW” present in the sample, both bound and unbound. The assay is capable of accurately measuring total LIF levels between, but not limited to 20 pg/ml (1 pM) and 2 ng/ml (100 pM). Total LIF levels can serve as an important treatment indicator, and allow clinicians to closely monitor the PK/PD dynamics of a LIF therapeutic antibody in an individual.

In a certain aspect described herein is a Leukemia Inhibitory Factor (LIF) complex, the complex comprising: LIF, a LIF capture antibody that specifically binds to LIF, a LIF detecting antibody that specifically binds to LIF, and optionally a LIF therapeutic antibody that specifically binds LIF, wherein the LIF detecting or the LIF capture antibody comprises A4 or a LIF binding fragment thereof. In certain embodiments, use of the LIF complex is an in vitro assay to quantify LIF. In certain embodiments, the LIF capture antibody or the LIF detecting antibody does not compete for binding with the LIF therapeutic antibody. In certain embodiments, the LIF is human LIF. In certain embodiments, the LIF therapeutic antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the LIF therapeutic antibody comprises: (a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and (b) an immunoglobulin light chain variable region (VL) sequence with an amino acid sequence at least about 80%, 90%, 95%, 97%, 98%, or 99% identical to the amino acid sequence set forth in any one of SEQ ID NOs: 18-21. In certain embodiments, the LIF therapeutic antibody comprises: (a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and (b) an immunoglobulin light chain sequence with an amino acid sequence at least about 80%, 90%, 95%, 97%, 98%, or 99% identical to the amino acid sequence set forth in any one of SEQ ID NOs: 34-37. In certain embodiments, the LIF capture antibody is coupled to a surface. In certain embodiments, the surface comprises an electrically conductive substance. In certain embodiments, the electrically conductive substance is an electrode. In certain embodiments, the LIF detecting antibody is coupled to a detectable moiety. In certain embodiments, the detectable moiety that generates a chemical signal, an electrochemical signal, a luminescent signal, or a fluorescent signal. In certain embodiments, the detectable moiety generates an electrochemical signal. In certain embodiments, the LIF detecting antibody and the LIF capture antibody do not bind to a region of LIF that physically interacts with gp130. In certain embodiments, the LIF capture antibody of the LIF detecting antibody comprises: (a) an immunoglobulin heavy chain variable region sequence with an amino acid sequence at least about 80%, 90%, 95%, 97%, 98%, or 99% identical to the amino acid sequence set forth in SEQ ID NO: 41; and (b) an immunoglobulin light chain variable region sequence with an amino acid sequence at least about 80%, 90%, 95%, 97%, 98%, or 99% identical to the amino acid sequence set forth in SEQ ID NO: 42. In certain embodiments, the complex is in a fluid. In certain embodiments, the complex is contained in at least one well of a multi-well plate. In certain embodiments, the complex is contained in at least one well of a 96-well plate, a 384-well plate, or a 1536-well plate. In certain embodiments, the complex is detectable at a level of 1 nanogram per milliliter. In certain embodiments, the assay has internal variability of less than 20% or 10%.

In another aspect, described herein, is a method of quantifying Leukemia Inhibitory Factor (LIF) in a sample from an individual comprising LIF comprising: (a) contacting the sample comprising LIF to a capture antibody that specifically binds to LIF; (b) contacting the sample comprising LIF to a detecting antibody that specifically binds LIF; (c) detecting the LIF in the sample that is bound to the capture antibody and the detecting antibody. In certain embodiments, the method is performed in vitro. In certain embodiments, the LIF is human LIF. In certain embodiments, the individual has been treated with a LIF therapeutic antibody. In certain embodiments, the LIF therapeutic antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the LIF therapeutic antibody comprises: (a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and (b) an immunoglobulin light chain variable region (VL) sequence with an amino acid sequence at least about 80%, 90%, 95%, 97%, 98%, or 99% identical to the amino acid sequence set forth in any one of SEQ ID NOs: 18-21. In certain embodiments, the LIF therapeutic antibody comprises: (a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and (b) an immunoglobulin light chain sequence with an amino acid sequence at least about 80%, 90%, 95%, 97%, 98%, or 99% identical to the amino acid sequence set forth in any one of SEQ ID NOs: 34-37. In certain embodiments, the LIF capture antibody is coupled to a surface. In certain embodiments, surface comprises an electrically conductive substance. In certain embodiments, the electrically conductive substance is an electrode. In certain embodiments, the LIF detecting antibody is coupled to a detectable moiety. In certain embodiments, the detectable moiety that generates a chemical signal, an electrochemical signal, a luminescent signal, or a fluorescent signal. In certain embodiments, the detectable moiety that generates an electrochemical signal. In certain embodiments, the LIF capture antibody and the LIF detecting antibody do not bind to a region of LIF that physically interacts with gp130. In certain embodiments, the LIF capture antibody or the LIF detecting antibody comprises: (a) an immunoglobulin heavy chain variable region sequence with an amino acid sequence at least about 80%, 90%, 95%, 97%, 98%, or 99% identical to the amino acid sequence set forth in SEQ ID NO: 41; and (b) an immunoglobulin light chain variable region sequence with an amino acid sequence at least about 80%, 90%, 95%, 97%, 98%, or 99% identical to the amino acid sequence set forth in SEQ ID NO: 42. In certain embodiments, the sample comprising LIF is in a fluid. In certain embodiments, the sample comprising LIF is contained in at least one well of a multi-well plate. In certain embodiments, the sample comprising LIF is contained in at least one well of a 96-well plate, a 384-well plate, or a 1536-well plate. In certain embodiments, the LIF is detectable at a level of 1 nanogram per milliliter. In certain embodiments, the assay has internal variability of less than 20%. In certain embodiments, the individual is a human individual. In certain embodiments, the method further comprises quantifying the LIF in the sample. In certain embodiments, the method further comprises transmitting a report comprising information on a quantity of LIF in the sample.

In another aspect, described herein, is a method of treating an individual with cancer comprising: (a) administering to the individual an initial dose of an antibody that binds Leukemia Inhibitory Factor (LIF); (b) determining a post-initial dose level of Leukemia Inhibitory Factor (LIF) in a sample from the individual with cancer. In certain embodiments, the method further comprises administering a subsequent dose of the antibody that binds Leukemia Inhibitory Factor (LIF). In certain embodiments, determining a post initial dose level of LIF is performed by a method according to this disclosure. In certain embodiments, the LIF therapeutic antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8;(d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the LIF therapeutic antibody comprises: (a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and (b) an immunoglobulin light chain variable region (VL) sequence with an amino acid sequence at least about 80%, 90%, 95%, 97%, 98%, or 99% identical to the amino acid sequence set forth in any one of SEQ ID NOs: 18-21. In certain embodiments, the LIF therapeutic antibody comprises: (a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and (b) an immunoglobulin light chain sequence with an amino acid sequence at least about 80%, 90%, 95%, 97%, 98%, or 99% identical to the amino acid sequence set forth in any one of SEQ ID NOs: 34-37. In certain embodiments, the post-initial dose level of LIF is not increased compared to a pre-initial dose level of LIF in the individual, and wherein the subsequent dose is administered at an increased amount compared to the initial dose. In certain embodiments, the post- initial dose level of LIF is increased by 2-fold or less compared to a pre-initial dose level of LIF in the individual, and wherein the subsequent dose is administered at an increased amount compared to the initial dose. In certain embodiments, the post- initial dose level of LIF is increased by 2-fold or less compared to a pre-initial dose level of LIF in the individual, and wherein the subsequent dose is administered at an increased amount compared to the initial dose. In certain embodiments, the post- initial dose level of LIF is not increased compared to a pre-initial dose level of LIF in the individual, and wherein the subsequent dose is administered at an earlier point in a treatment schedule. In certain embodiments, the post- initial dose level of LIF is increased by 2-fold or less compared to a pre-initial dose level of LIF in the individual, and wherein the subsequent dose is administered at an earlier point in a treatment schedule. In certain embodiments, the post-initial dose level of LIF is increased by 2-fold or less compared to a pre-initial dose level of LIF in the individual, and wherein the subsequent dose is administered at an earlier point in a treatment schedule. In certain embodiments, the initial dose is a first dose of the antibody that binds Leukemia Inhibitory Factor (LIF). In certain embodiments, the initial dose is any dose in a plurality of doses of the antibody that binds Leukemia Inhibitory Factor (LIF).

In another aspect, described herein, is a method of treating an individual with cancer comprising: (a) administering to the individual an initial dose comprising an antibody that binds Leukemia Inhibitory Factor (LIF); (b) receiving a post-initial dose level of Leukemia Inhibitory Factor (LIF) in a sample from the individual with cancer. In certain embodiments, the method further comprises administering a subsequent dose of the antibody that binds Leukemia Inhibitory Factor (LIF). In certain embodiments determining a post initial dose level of LIF is performed by a method according to this disclosure. In certain embodiments, the LIF therapeutic antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8;(d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. In certain embodiments, the LIF therapeutic antibody comprises: (a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and (b) an immunoglobulin light chain variable region (VL) sequence with an amino acid sequence at least about 80%, 90%, 95%, 97%, 98%, or 99% identical to the amino acid sequence set forth in any one of SEQ ID NOs: 18-21. In certain embodiments, the LIF therapeutic antibody comprises: (a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and (b) an immunoglobulin light chain sequence with an amino acid sequence at least about 80%, 90%, 95%, 97%, 98%, or 99% identical to the amino acid sequence set forth in any one of SEQ ID NOs: 34-37. In certain embodiments, the post-initial dose level of LIF is not increased compared to a pre-initial dose level of LIF in the individual, and wherein the subsequent dose is administered at an increased amount compared to the initial dose. In certain embodiments, the post- initial dose level of LIF is increased by 2-fold or less compared to a pre-initial dose level of LIF in the individual, and wherein the subsequent dose is administered at an increased amount compared to the initial dose. In certain embodiments, the post- initial dose level of LIF is increased by 2-fold or less compared to a pre-initial dose level of LIF in the individual, and wherein the subsequent dose is administered at an increased amount compared to the initial dose. In certain embodiments, the post- initial dose level of LIF is not increased compared to a pre-initial dose level of LIF in the individual, and wherein the subsequent dose is administered at an earlier point in a treatment schedule. In certain embodiments, the post- initial dose level of LIF is increased by 2-fold or less compared to a pre-initial dose level of LIF in the individual, and wherein the subsequent dose is administered at an earlier point in a treatment schedule. In certain embodiments, the post- initial dose level of LIF is increased by 2-fold or less compared to a pre-initial dose level of LIF in the individual, and wherein the subsequent dose is administered at an earlier point in a treatment schedule. In certain embodiments, the initial dose is a first dose of the antibody that binds Leukemia Inhibitory Factor (LIF). In certain embodiments, the initial dose is any dose in a plurality of doses of the antibody that binds Leukemia Inhibitory Factor (LIF).

In another aspect described herein is a Leukemia Inhibitory Factor (LIF) binding antibody or fragment thereof, wherein the LIF binding antibody or fragment thereof comprises: an immunoglobulin heavy chain variable region sequence with an amino acid sequence at least about 90% identical to the amino acid sequence set forth in SEQ ID NO: 41; and an immunoglobulin light chain variable region sequence with an amino acid sequence at least about 90% identical to the amino acid sequence set forth in SEQ ID NO: 42. In some embodiments, the LIF binding antibody or fragment thereof comprises: an immunoglobulin heavy chain variable region sequence with an amino acid sequence at least about 95% identical to the amino acid sequence set forth in SEQ ID NO: 41; and an immunoglobulin light chain variable region sequence with an amino acid sequence at least about 95% identical to the amino acid sequence set forth in SEQ ID NO: 42. In some embodiments, the LIF binding antibody or fragment thereof comprises: an immunoglobulin heavy chain variable region sequence with an amino acid sequence identical to the amino acid sequence set forth in SEQ ID NO: 41; and an immunoglobulin light chain variable region sequence with an amino acid sequence identical to the amino acid sequence set forth in SEQ ID NO: 42. In some embodiments, the use of the LIF binding antibody is an in vitro assay to quantify LIF. In some embodiments, the LIF binding antibody is coupled to a detectable moiety. In some embodiments, the detectable moiety that generates a chemical signal, an electrochemical signal, a luminescent signal, or a fluorescent signal. In some embodiments, the detectable moiety generates an electrochemical signal.

In some aspects, described herein comprises a Leukemia Inhibitory Factor (LIF) complex, the complex comprising: LIF, a LIF capture antibody that specifically binds to LIF, a LIF detecting antibody that specifically binds to LIF, and optionally a LIF therapeutic antibody that specifically binds LIF, wherein the LIF detecting or the LIF capture antibody comprises A4 or a LIF binding fragment thereof In certain embodiments, the LIF capture antibody or the LIF detecting antibody does not compete for binding with the LIF therapeutic antibody. In certain embodiments, the LIF therapeutic antibody comprises: an immunoglobulin heavy chain complementarity determining region 1 (VH-CDR1) comprising the amino acid sequence set forth in any one of SEQ ID NOs: 1-3;an immunoglobulin heavy chain complementarity determining region 2 (VH-CDR2) comprising the amino acid sequence set forth in any one of SEQ ID NOs: 4 or 5; an immunoglobulin heavy chain complementarity determining region 3 (VH-CDR3) comprising the amino acid sequence set forth in any one of SEQ ID NOs: 6-8;an immunoglobulin light chain complementarity determining region 1 (VL-CDR1) comprising the amino acid sequence set forth in any one of SEQ ID NOs: 9 or 10; an immunoglobulin light chain complementarity determining region 2 (VL-CDR2) comprising the amino acid sequence set forth in any one of SEQ ID NOs: 11 or 12; and an immunoglobulin light chain complementarity determining region 3 (VL-CDR3) comprising the amino acid sequence set forth in SEQ ID NO: 13.In certain embodiments, the LIF therapeutic antibody comprises: an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and an immunoglobulin light chain variable region (VL) sequence with an amino acid sequence at least about 80%, 90%, 95%, 97%, 98%, or 99% identical to the amino acid sequence set forth in any one of SEQ ID NOs: 18-21.In certain embodiments, the LIF therapeutic antibody comprises: an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and an immunoglobulin light chain sequence with an amino acid sequence at least about 80%, 90%, 95%, 97%, 98%, or 99% identical to the amino acid sequence set forth in any one of SEQ ID NOs: 34-37.In certain embodiments, the LIF capture antibody is coupled to a surface, wherein the surface comprises an electrically conductive substance, or wherein the electrically conductive substance is an electrode. In certain embodiments, the LIF detecting antibody is coupled to a detectable moiety, wherein the detectable moiety that generates a chemical signal, an electrochemical signal, a luminescent signal, or a fluorescent signal, or wherein the detectable moiety generates an electrochemical signal In certain embodiments, the LIF detecting antibody and the LIF capture antibody do not bind to a region of LIF that physically interacts with gp130.In certain embodiments, the LIF capture antibody of the LIF detecting antibody comprises: an immunoglobulin heavy chain variable region sequence with an amino acid sequence at least about 80%, 90%, 95%, 97%, 98%, or 99% identical to the amino acid sequence set forth in SEQ ID NO: 41; and an immunoglobulin light chain variable region sequence with an amino acid sequence at least about 80%, 90%, 95%, 97%, 98%, or 99% identical to the amino acid sequence set forth in SEQ ID NO: 42. In certain embodiments, the complex is contained in at least one well of a multi-well plate, wherein the complex is contained in at least one well of a 96-well plate, a 384-well plate, or a 1536-well plate, or wherein the complex is detectable at a level of 1 nanogram per milliliter.

In another aspect, described herein comprises a method of quantifying Leukemia Inhibitory Factor (LIF) in a sample from an individual comprising LIF comprising: contacting the sample comprising LIF to a capture antibody that specifically binds to LIF; contacting the sample comprising LIF to a detecting antibody that specifically binds LIF; detecting the LIF in the sample that is bound to the capture antibody and the detecting antibody; wherein the LIF detecting or the LIF capture antibody comprises A4 or a LIF binding fragment thereof . In some embodiments, the individual has been treated with a LIF therapeutic antibody. In some embodiments, the LIF therapeutic antibody comprises: an immunoglobulin heavy chain complementarity determining region 1 (VH-CDR1) comprising the amino acid sequence set forth in any one of SEQ ID NOs: 1-3;an immunoglobulin heavy chain complementarity determining region 2 (VH-CDR2) comprising the amino acid sequence set forth in any one of SEQ ID NOs: 4 or 5; an immunoglobulin heavy chain complementarity determining region 3 (VH-CDR3) comprising the amino acid sequence set forth in any one of SEQ ID NOs: 6-8;an immunoglobulin light chain complementarity determining region 1 (VL-CDR1) comprising the amino acid sequence set forth in any one of SEQ ID NOs: 9 or 10; an immunoglobulin light chain complementarity determining region 2 (VL-CDR2) comprising the amino acid sequence set forth in any one of SEQ ID NOs: 11 or 12; and an immunoglobulin light chain complementarity determining region 3 (VL-CDR3) comprising the amino acid sequence set forth in SEQ ID NO: 13.In some embodiments, the LIF therapeutic antibody comprises: an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and an immunoglobulin light chain variable region (VL) sequence with an amino acid sequence at least about 80%, 90%, 95%, 97%, 98%, or 99% identical to the amino acid sequence set forth in any one of SEQ ID NOs: 18-21. In some embodiments, the LIF therapeutic antibody comprises: an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and an immunoglobulin light chain sequence with an amino acid sequence at least about 80%, 90%, 95%, 97%, 98%, or 99% identical to the amino acid sequence set forth in any one of SEQ ID NOs: 34-37.In some embodiments, the LIF capture antibody is coupled to a surface, wherein the surface comprises an electrically conductive substance, or wherein the electrically conductive substance is an electrode. In some embodiments, the LIF detecting antibody is coupled to a detectable moiety, wherein the detectable moiety that generates a chemical signal, an electrochemical signal, a luminescent signal, or a fluorescent signal, or wherein the detectable moiety that generates an electrochemical signal. In some embodiments, the LIF capture antibody and the LIF detecting antibody do not bind to a region of LIF that physically interacts with gp130. In some embodiments, the LIF capture antibody or the LIF detecting antibody comprises: an immunoglobulin heavy chain variable region sequence with an amino acid sequence at least about 80%, 90%, 95%, 97%, 98%, or 99% identical to the amino acid sequence set forth in SEQ ID NO: 41; and an immunoglobulin light chain variable region sequence with an amino acid sequence at least about 80%, 90%, 95%, 97%, 98%, or 99% identical to the amino acid sequence set forth in SEQ ID NO: 42.In some embodiments, the sample comprising LIF is contained in at least one well of a multi-well plate, or wherein the sample comprising LIF is contained in at least one well of a 96-well plate, a 384-well plate, or a 1536-well plate.

In another aspect, described herein comprises a Leukemia Inhibitory Factor (LIF) binding antibody or fragment thereof, wherein the LIF binding antibody or fragment thereof comprises: an immunoglobulin heavy chain variable region with an amino acid sequence at least about 90% identical to the amino acid sequence set forth in SEQ ID NO: 41; and an immunoglobulin light chain variable region with an amino acid sequence at least about 90% identical to the amino acid sequence set forth in SEQ ID NO: 42. In some embodiments, the immunoglobulin heavy chain variable region comprises an amino acid sequence at least about 95% identical to the amino acid sequence set forth in SEQ ID NO: 41; and the immunoglobulin light chain variable region comprises an amino acid sequence at least about 95% identical to the amino acid sequence set forth in SEQ ID NO: 42. In some embodiments, the immunoglobulin heavy chain variable region comprises the amino acid sequence set forth in SEQ ID NO: 41; and the immunoglobulin light chain variable region comprises the amino acid sequence set forth in SEQ ID NO: 42. In some embodiments the LIF binding antibody is coupled to a detectable moiety, wherein the detectable moiety generates a chemical signal, an electrochemical signal, a luminescent signal, or a fluorescent signal, or wherein the detectable moiety generates an electrochemical signal. In some embodiments, the LIF binding antibody is specifically bound to LIF.

### BRIEF DESCRIPTION OF THE DRAWINGS

FIG. 1 depicts a western blot showing inhibition of LIF-induced STAT3 phosphorylation of different anti-LIF humanized antibodies.

FIG. 2A and 2B depicts a western blot showing inhibition of LIF-induced STAT3 phosphorylation humanized and parental 5D8 antibody.

FIG. 3A shows an IC₅₀ for LIF inhibition in U-251 cells using the h5D8 antibody.

FIG. 3B shows representative IC₅₀ dose response curves of r5D8 and h5D8 inhibition of pSTAT3 under endogenous LIF stimulation conditions. Shown are the representative curves (n=1 h5D8, n=2 r5D8).

FIG. 4 depicts a western blot showing inhibition of LIF-induced STAT3 phosphorylation of different monoclonal antibodies described in this disclosure.

FIG. 5 depicts immunohistochemistry staining and quantitation of LIF expression in glioblastoma multiforme (GBM), NSCLC (non-small cell lung carcinoma), ovarian cancer, colorectal cancer tumors, and pancreatic tumors from human patients. Bars represent mean+/−SEM.

FIG. 6 is a graph showing an experiment conducted in a mouse model of non-small cell lung cancer using the humanized 5D8 antibody.

FIG. 7A shows the effect of r5D8 on inhibition of U251 cells in an orthotopic mouse model of GBM. Quantitation shown at day 26.

FIG. 7B shows data from mice inoculated with luciferase expressing human U251 GBM cells and then treated with 100, 200 or 300 μg of h5D8 or vehicle twice a week. Tumor size was determined by bioluminescence (Xenogen IVIS Spectrum) on day 7. The graph shows individual tumor measurements with horizontal bars indicating mean±SEM. Statistical significance was calculated using the unpaired non-parametric Mann-Whitney U-test.

FIG. 8A shows the effect of r5D8 on inhibition of growth of ovarian cancer cells in a syngeneic mouse model.

FIG. 8B shows the individual measurements of tumors at day 25.

FIG. 8C illustrates that h5D8 shows a significant reduction in tumor growth when administered at 200 μg/mouse twice weekly (p<0.05). Symbols are mean+SEM, statistical significance compared with vehicle (with unpaired non-parametric Mann-Whitney U-test).

FIG. 9A shows the effect of r5D8 on inhibition of growth of colorectal cancer cells in a syngeneic mouse model.

FIG. 9B shows the individual measurements of tumors at day 17.

FIG. 10A shows reduction of macrophage infiltration to tumor sites in an orthotopic mouse model of GBM with a representative image and quantitation of CCL22+ cells.

FIG. 10B shows reduction of macrophage infiltration in a human organotypic tissue slice culture model. Shown are a representative image (left) and quantitation (right).

FIG. 10C shows reduction of macrophage infiltration to tumor sites in a syngeneic mouse model of ovarian cancer with a representative image and quantitation of CCL22+ cells.

FIG. 10D shows reduction of macrophage infiltration to tumor sites in a syngeneic mouse model of colorectal cancer with a representative image and quantitation of CCL22+ cells.

FIG. 11A shows increases in non-myeloid effector cells in a syngeneic mouse model of ovarian cancer after treatment with r5D8.

FIG. 11B shows increases in non-myeloid effector cells in a syngeneic mouse model of colorectal cancer after treatment with r5D8.

FIG. 11C shows decreases in percentage of CD4+ T_{REG} cells in a mouse model of NSCLC cancer after treatment with r5D8.

FIG. 12 shows data from mice bearing CT26 tumors treated twice weekly with PBS (control) or r5D8 administered intraperitoneally in the presence or absence of anti-CD4 and anti-CD8 depleting antibodies. The graph shows individual tumor measurements at d13 expressed as mean tumor volume+SEM. Statistical differences between groups was determined by unpaired non-parametric Mann-Whitney U-test. R5D8 inhibited the growth of CT26 tumors (*p<0.05). The tumor growth inhibition by r5D8 was significantly reduced in the presence of anti-CD4 and anti-CD8 depleting antibodies (****p<0.0001).

FIG. 13A illustrates an overview of the co-crystal structure of h5D8 Fab in complex with LIF. The gp130 interacting site is mapped on the surface of LIF (dark shaded).

FIG. 13B illustrates detailed interactions between LIF and h5D8, showing residues forming salt bridges and h5D8 residues with buried surface areas greater than 100 AÅ².

FIG. 14A illustrates superposition of the five h5D8 Fab crystal structures and indicates a high degree of similarity despite being crystallized in different chemical conditions.

FIG. 14B illustrates an extensive network of Van der Waals interactions mediated by unpaired Cys100. This residue is well-ordered, partakes in shaping the conformations of HCDR1 and HCDR3 and is not involved in undesired disulfide scrambling. Distances between residues are shown as dashed lines and labeled.

FIG. 15A illustrates binding of h5D8 C100 mutants to human LIF by ELISA.

FIG. 15B illustrates binding of h5D8 C100 mutants to mouse LIF by ELISA.

FIG. 16A illustrates that h5D8 does not block binding between LIF and LIFR by Octet. Sequential binding of h5D8 to LIF followed by LIFR.

FIG. 16B and 16C illustrate ELISA analysis of LIF/mAb complexes binding to immobilized LIFR or gp130. Signals of species-specific peroxidase conjugated anti-IgG antibodies (anti-human for (-) and h5D8, anti-rat for r5d8 and B09) detecting the antibody portion of mAb/LIF complexes binding immobilized LIFR (FIG. 16B) or gp130 (FIG. 16C) coated plates.

FIG. 17A and 17B illustrate mRNA expression of LIF (FIG. 17A) or LIFR (FIG. 17B) in 72 different human tissues.

FIG. 18 shows a schematic of the total LIF assay described herein.

FIG. 19A demonstrates the signal detection curve of the total LIF assay over a wide range of concentrations of total LIF bound to a therapeutic antibody.

FIG. 19B depicts the signal detection curve of the total LIF level assay of from 20 pg/ml to 1.25 ng/ml total LIF bound to a therapeutic antibody which overlaps, but is not limited to the range of potential levels of total LIF in human plasma or serum of patients given a therapeutic anti-LIF antibody.

FIGS. 20A and 20B demonstrates the uniformity of the total LIF level assay of from 20 pg/ml to 1.25 ng/m1 total LIF bound to a therapeutic antibody in human serum from six different donors both individually (FIG. 20A) and overlaid (FIG. 20B).

FIG. 21 shows that the total LIF assay is stable over a range of concentrations between 10 ug/ml to 810 ug/ml of a therapeutic anti-LIF mAb (h5D8) that binds an epitope overlapping with the gp130 binding site of LIF.

FIG. 22A depicts optimization testing for diluents that might reduce the HAMA interference that was observed with some serum samples. HAMA (human anti-mouse antibodies) are anti-animal antibodies that cross-react with the capture and detection reagents to produce a false positive signal, or high background.

FIG. 22B demonstrates the performance comparison of two candidate diluents during the optimization phase of assay development.

FIG. 23 shows optimization testing of different spot-coating concentrations of the capture antibody.

FIG. 24 shows optimization testing of different capture antibodies. mAb1=rabbit monoclonal A4 antibody; m mAb2=Creative Diagnostics; rat monoclonal antibody, clone JNH4008E21; Cat. No. DCABH-3367; mAb3=chimeric rabbit/human A4 antibody.

FIGS. 25 A-D show ELISA analysis of LIF/mAb complexes binding to immobilized LIFR or gp130. (FIGS. 25A and B) Signals of species-specific peroxidase conjugated anti-IgG antibodies detecting the antibody portion of mAb/LIF complexes binding immobilized LIFR (FIG. 25A) or gp130 (FIG. 25B) coated plates. (FIGS. 25C and D) Signals of Avidin-HRP detecting the Bt-LIF portion of mAb/Bt-LIF complexes binding immobilized LIFR (FIG. 25C) or gp130 (FIG. 25D) coated plates. [1C7 binds LIFR and blocks LIF binding. 28105 binds gp130 and blocks LIF binding. B09 binds an epitope of LIF outside of the LIFR and gp130 binding sites].

FIGS. 26 A-C show the LIF level relative to time of 1st dose in three subjects: Subject A (FIG. 26A), Subject B (FIG. 26B), Subject C (FIG. 26C).

### DETAILED DESCRIPTION

In one aspect, described herein, is a Leukemia Inhibitory Factor (LIF) complex, the complex comprising: LIF; a LIF capture antibody that specifically binds to LIF; a LIF detecting antibody that specifically binds to LIF; and optionally a LIF therapeutic antibody that specifically binds LIF.

In another aspect, described herein, is a method of quantifying Leukemia Inhibitory Factor (LIF) in a sample from an individual comprising: (a) contacting the sample comprising LIF to a capture antibody that specifically binds to LIF; (b) contacting the sample comprising LIF to a detecting antibody that specifically binds LIF; and (c) detecting the LIF in the sample that is bound to the capture antibody and the detecting antibody.

In another aspect, described herein, is a method of treating an individual with cancer comprising: (a) administering to the individual an initial dose of an antibody that binds Leukemia Inhibitory Factor (LIF); (b) determining a post-treatment level of Leukemia Inhibitory Factor (LIF) in a sample from the individual with cancer.

In another aspect, described herein, is a method of treating an individual with cancer comprising: (a) administering to the individual an initial dose comprising an antibody that binds Leukemia Inhibitory Factor (LIF); (b) receiving a post-treatment level of Leukemia Inhibitory Factor (LIF) in a sample from the individual with cancer.

As used herein the terms “individual,” “subject,” and “patient” are used interchangeably and include humans diagnosed with or suspected of being afflicted with a tumor, a cancer, or other neoplasm.

As used herein, unless otherwise indicated, the term “antibody” includes antigen binding fragments of antibodies, i.e. antibody fragments that retain the ability to bind specifically to the antigen bound by the full-length antibody, e.g. fragments that retain one or more CDR regions. Examples of antibody fragments include, but are not limited to, Fab, Fab′, F(ab′)2, and Fv fragments; diabodies; linear antibodies; heavy chain antibodies, single-chain antibody molecules, e.g. single-chain variable region fragments (scFv), nanobodies and multispecific antibodies formed from antibody fragments with separate specificities, such as a bispecific antibody. In certain embodiments, the antibodies are humanized in such a way as to reduce an individual's immune response to the antibody. For example, the antibodies may be chimeric, e.g. non-human variable region with human constant region, or CDR grafted, e.g. non-human CDR regions with human constant region and variable region framework sequences. In certain embodiments, antibodies are deimmunized after humanization. Deimmunization involves removing or mutating one or more T-cell epitopes in the constant region of the antibody. In certain embodiments, the antibodies described herein are monoclonal. As used herein a “recombinant antibody” is an antibody that comprises an amino acid sequence derived from two different species or, or two different sources, and includes synthetic molecules, for example, an antibody that comprises a non-human CDR and a human framework or constant region. In certain embodiments, recombinant antibodies of the present invention are produced from a recombinant DNA molecule or synthesized. The terms “cancer” and “tumor” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion, and metastasis formation are also termed malignant properties of cancers. These malignant properties differentiate cancers from benign tumors, which typically do not invade or metastasize.

As used herein a “therapeutic antibody” is one administered to an individual and intended to produce one or more beneficial effects useful in the treatment of cancer. Therapeutic antibodies of the current disclosure include antibodies that have CDR sequences identical to h5D8, or CDRs that vary from h5D8 but that possess similar binding characteristics (epitope, affinity, or biological effect) and can produce one or more beneficial effects useful to treat cancer.

As used herein “reference level” refers to a level of LIF protein detected in a biological sample that corresponds to a level of LIF corresponding to an elevated level that is consistent with therapeutic antibody engagement with the target (e.g., LIF). A reference level can be defined based on a population of individuals that has been treated and corresponds to a level that indicates maximal target engagement in at least 50%, 55%, 60%, 65%, 70%, 75%, 80%, 85%, 90%, 95% of the individuals in the population.

Percent (%) sequence identity with respect to a reference polypeptide or antibody sequence is the percentage of amino acid residues in a candidate sequence that are identical with the amino acid residues in the reference polypeptide or antibody sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN, or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to Unless specifically stated otherwise, all % amino acid sequence identity values used herein are obtained as described in the immediately preceding paragraph using the ALIGN-2 computer program.

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that directly contact the antigen binding protein. Most often, epitopes reside on proteins, but in some instances can reside on other kinds of molecules, such as saccharides or lipids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules. Structural attributes of the antibodies described herein

A complementarity determining region (“CDR”) is a part of an immunoglobulin (antibody) variable region that is primarily responsible for the antigen binding specificity of the antibody. CDR regions are highly variable from one antibody to the next even when the antibody specifically binds the same target or epitope. A heavy chain variable region comprises three CDR regions, abbreviated VH-CDR1, VH-CDR2, and VH-CDR3; and a light chain variable region comprises three CDR regions, abbreviated VL-CDR1, VL-CDR2, and VL-CDR3. These CDR regions are ordered consecutively in the variable region with the CDR1 being the most N-terminal and the CDR3 being the most C-terminal. Interspersed between the CDRs are framework regions which contribute to the structure and display much less variability than the CDR regions. A heavy chain variable region comprises four framework regions, abbreviated VH-FR1, VH-FR2, VH-FR3, and VH-FR4; and a light chain variable region comprises four framework regions, abbreviated VL-FR1, VL-FR2, VL-FR3, and VL-FR4. Complete full-sized bivalent antibodies comprising two heavy and light chains will comprise: 12 CDRs, with three unique heavy chain CDRs and three unique light chain CDRs; 16 FR regions, with four unique heavy chain FR regions and four unique light chain FR regions. In certain embodiments, the antibodies described herein minimally comprise three heavy chain CDRs. In certain embodiments, the antibodies described herein minimally comprise three light chain CDRs. In certain embodiments, the antibodies described herein minimally comprise three heavy chain CDRs and three light chain CDRs. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” (“Contact” numbering scheme); Lefranc MP et al.,“IMGT unique numbering for immunoglobulin and T cell receptor variable domains and Ig superfamily V-like domains,” Dev Comp Immunol, 2003 Jan;27(1):55-77 (“IMGT” numbering scheme); and Honegger A and Pluckthun A, “Yet another numbering scheme for immunoglobulin variable domains: an automatic modeling and analysis tool,” J Mot Biol, 2001 Jun 8;309(3):657-70, (“Aho” numbering scheme). CDRs are identified herein from variable sequences provided using different numbering systems, herein with the Kabat, the IMGT, the Chothia numbering system, or any combination of the three. The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)). In certain embodiments, the antibodies described herein comprise variable regions of rat origin. In certain embodiments, the antibodies described herein comprise CDRs of rat origin. In certain embodiments, the antibodies described herein comprise variable regions of mouse origin. In certain embodiments, the antibodies described herein comprise CDRs of mouse origin.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex is analyzed to identify contact points between the antibody and antigen. Such contact residues and neighboring residues may be targeted or eliminated as candidates for substitution. Variants may be screened to determine whether they contain the desired properties.

In certain embodiments, the antibodies described herein comprise CDRs selected from any one or more of SEQ ID NO: 41 or SEQ ID NO: 42. In certain embodiments, the CDRs are selcted from the sequences using the Kabat, the IMGT, the Chothia numbering system, or any combination of the three. The boundaries of a given CDR or FR may vary depending on the scheme used for identification.

In certain embodiments, the antibodies described herein comprise a constant region in addition to a variable region. The heavy chain constant region (C_{H}) comprises four domains abbreviated C_{H}1, C_{H}2, C_{H}3, and C_{H}4, located at the C-terminal end of the full heavy chain polypeptide, C-terminal to the variable region. The light chain constant region (C_{L}) is much smaller than the C_{H} and is located at the C-terminal end of the full light chain polypeptide, C-terminal to the variable region. The constant region is highly conserved and comprises different isotypes that are associated with slightly different functions and properties. In certain embodiments, the constant region is dispensable for antibody binding to a target antigen. In certain embodiments, the constant regions of the antibody, both heavy and light chains are dispensable for antibody binding. In certain embodiments, the antibodies described herein lack one or more of a light chain constant region, heavy chain constant region, or both. Most monoclonal antibodies are of an IgG isotype; which is further divided into four subclasses IgG₁, IgG₂, IgG₃, and IgG₄. In certain embodiments, the antibodies described herein comprise any IgG subclass. In certain embodiments, the IgG subclass comprises IgG₁. In certain embodiments, the IgG subclass comprises IgG₂. In certain embodiments, the IgG subclass comprises IgG₃. In certain embodiments, the IgG subclass comprises IgG_{4.}

Antibodies comprise a fragment crystallizable region (Fc region) that is responsible for binding to complement and Fc receptors. The Fc region comprises the C_{H}2, C_{H}3, and C_{H}4 regions of the antibody molecule. The Fc region of an antibody is responsible for activating complement and antibody dependent cell cytotoxicity (ADCC). The Fc region also contributes to an antibody's serum half-life. In certain embodiments, the Fc region of the therapeutic antibodies described herein comprise one or more amino acid substitutions that promote complement mediated cell lysis. In certain embodiments, the Fc region of the therapeutic antibodies described herein comprises one or more amino acid substitutions that promote ADCC. In certain embodiments, the Fc region of the therapeutic antibodies described herein comprises one or more amino acid substitutions that reduce complement mediated cell lysis. In certain embodiments, the Fc region of the therapeutic antibodies described herein comprises one or more amino acid substitutions that increase binding of the antibody to an Fc receptor. In certain embodiments, the Fc receptor comprises FcγRI (CD64), FcγRIIA (CD32), FcγRIIIA (CD16a), FcγRIIIB (CD16b), or any combination thereof In certain embodiments, the Fc region of the the therapeutic antibodies described herein comprise one or more amino acid substitutions that increase the serum half-life of the antibody. In certain embodiments, the one or more amino acid substitutions that increase the serum half-life of the therapeutic antibody increase affinity of the antibody to the neonatal Fc receptor (FcRn).

Antibodies useful in the clinic are often “humanized” to reduce immunogenicity in human individuals. Humanized antibodies improve safety and efficacy of monoclonal antibody therapy. One common method of humanization is to produce a monoclonal antibody in any suitable animal (e.g., mouse, rat, hamster) and replace the constant region with a human constant region, antibodies engineered in this way are termed “chimeric”. Another common method is “CDR grafting” which replaces the non-human V-FRs with human V-FRs. In the CDR grafting method all residues except for the CDR region are of human origin. In certain embodiments, the antibodies described herein are humanized. In certain embodiments, the antibodies described herein are chimeric. In certain embodiments, the antibodies described herein are CDR grafted.

### Methods of Determining a Total LIF Level

In an unbound state most soluble cytokines have a short half-life in the blood, which leads to low-levels at steady-state. However, when bound by an antibody this half-life can increase, leading to higher steady-state levels. Assaying for levels of total LIF in a patient sample can be used to define peripheral target engagement (e.g., LIF binding by a therapeutic anti-LIF antibody), and to inform further doses of a LIF-binding therapeutic antibody. Thus, the methods described herein are useful for determining LIF levels in an individual in response to treatment after one or more doses of a therapeutic antibody that binds LIF. The method can be used in determining a response to treatment, and for adjusting subsequent treatments. In certain embodiments, a level of LIF can be determined before treatment with a LIF therapeutic antibody to serve as a base-line LIF level, wherein a treatment is expected to increase LIF levels in the patient. In certain embodiments, no base-line is obtained, and a treatment modification can be effected based upon an absolute level of LIF.

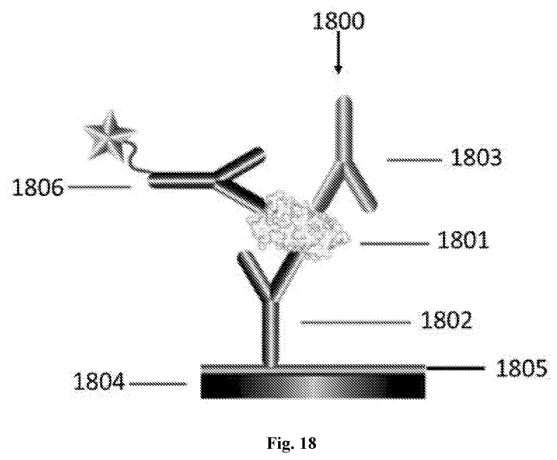

The LIF assay described herein measures LIF both bound and unbound by a therapeutic antibody, thus, determining a total LIF level. As shown in FIG. 18, the assay forms a molecular complex 1800, comprising LIF 1801, bound by a capture antibody 1802, which is immobilized to a surface 1804, optionally comprising an electrically conductive substance 1805 such that an electrical signal produced by the detecting antibody can be measured. A detecting antibody 1806 is then added. This detecting antibody can be coupled to, either directly or indirectly, a detectable moiety. In this complex, a therapeutic antibody 1803 can be bound to LIF 1801. Because capture antibody 1802 and detecting antibody 1806 bind to distinct portions of the LIF molecule the assay measures total LIF, either bound or unbound by a therapeutic antibody. For the purposes of the current assay, the therapeutic antibody is one that either possesses the CDR residues of 5D8 or has a similar binding region as an antibody with the 5D8 CDRs. This assay is also compatible with other therapeutic antibodies that bind the same or substantially the same portion of LIF. The region of LIF bound by an antibody with the 5D8 CDRs is detailed herein. While the residues bound by 5D8 herein are determined by crystal structure, any antibody that competes with 5D8 for binding can serve as the therapeutic antibody, as long as the therapeutic antibody does not compete for binding with either the capture or detection antibody. Further, any given therapeutic antibody may differ slightly in the LIF amino acid residues bound when compared to 5D8, such that 1, 2, 3, 4, 5, 6, 7, 8, 9, 10, 11, 12, 13, 14, 15, 16, 17, 18, 19, or more residues of the 5D8 epitope may be bound.

The general steps of conducting the assay comprise: 1) adsorbing or conjugating a capture antibody to a substrate; 2) incubating the substrate conjugated with capture antibody with a biological sample comprising LIF; 3) incubating the capture antibody-LIF complex formed with a LIF detecting antibody; and 4) measuring a signal produced from the detecting antibody. LIF levels can then be quantitated by comparison to a standard curve of known LIF concentration. Exact incubation parameters are variable but will generally range from 30 minutes to 120 minutes at room temperature, or 2 hours to overnight at 4° C., although longer incubation times are compatible with the assay. In some embodiments, the incubation parameters may range from 1 minute to 30 minutes at 37° C., 1 minute to 30 minutes at room temperature, 120 minutes to 240 minutes at room temperature, 30 minutes to 2 hours at 4° C., or 8 hours to 24 hours at 4° C. Wash steps can be added after any of the steps to remove excess unbound antibody or plasma/serum components. In certain embodiments, a wash buffer comprises detergent or salt concentrations that reduce non-specific binding. In certain embodiments, depending on the detectable moiety the measuring step may comprise an additional step of adding a substrate for the detectable moiety to convert into a detectable signal.

In certain embodiments, the LIF capture antibody is coupled to a surface. In certain embodiments, the surface comprises an electrically conductive substance. In certain embodiments, the electrically conductive substance is an electrode. In certain embodiments, the surface is a substrate. In some embodiments, the substrate is derived from natural sources. In some embodiments, the substrate is synthetic. In some embodiments, the substrate derived from natural sources is extracellular matrix (“ECM”). In some embodiments, the ECM comprises collagen, fibronectin, laminin, or a combination thereof. In some embodiments, the ECM comprises a hydrogel. In certain embodiments, the synthetic substrate comprises poly-L-lysine.

The assay described herein can be performed on various types of biological samples collected from an individual treated with or to be treated with a therapeutic antibody. The biological sample can be a tissue sample, a tumor biopsy, a blood sample, or a urine sample. In certain embodiments, the sample is a blood sample. In certain embodiments, the sample is a plasma sample. In certain embodiments, the sample is a serum sample. The sample may be diluted prior to addition to the assay.

The assay described herein can be used to determine an individual's response to treatment with a therapeutic antibody. The assay can be conducted on a biological sample from the individual before a therapeutic antibody has been administered, after a therapeutic antibody has been administered, or both before and after a therapeutic antibody has been administered. The assay can be conducted before an initial dose of an antibody that binds Leukemia Inhibitory Factor (LIF). The assay can be conducted after an initial dose of an antibody that binds Leukemia Inhibitory Factor (LIF). The assay can be conducted after a post-initial dose of an antibody that binds LIF. In certain embodiments, the post-initial dose level of LIF is increased from the pre-initial dose level of LIF by 2 -fold or less. In certain embodiments, the post-initial dose level of LIF is increased from the pre-initial dose level of LIF by 2 -fold to 10 -fold. In certain embodiments, the post-initial dose level of LIF is increased from the pre-initial dose level of LIF by 2 -fold to 3 -fold, 2 -fold to 4 -fold, 2 -fold to 5 -fold, 2 -fold to 6 -fold, 2 -fold to 7 -fold, 2 -fold to 8 -fold, 2 -fold to 9 -fold, 2 -fold to 10 -fold, 3 -fold to 4 -fold, 3 -fold to 5-fold, 3 -fold to 6 -fold, 3 -fold to 7 -fold, 3 -fold to 8 -fold, 3 -fold to 9 -fold, 3 -fold to 10 -fold, 4 -fold to 5 -fold, 4 -fold to 6 -fold, 4 -fold to 7 -fold, 4 -fold to 8 -fold, 4 -fold to 9 -fold, 4 -fold to 10 -fold, 5 -fold to 6 -fold, 5 -fold to 7 -fold, 5 -fold to 8 -fold, 5 -fold to 9 -fold, 5 -fold to 10 -fold, 6 -fold to 7 -fold, 6 -fold to 8 -fold, 6 -fold to 9 -fold, 6 -fold to 10 -fold, 7 -fold to 8 -fold, 7 -fold to 9 -fold, 7 -fold to 10 -fold, 8 -fold to 9 -fold, 8 -fold to 10 -fold, or 9 -fold to 10 -fold. In certain embodiments, the post-initial dose level of LIF is increased from the pre-initial dose level of LIF by 2 -fold, 3 -fold, 4 -fold, 5 -fold, 6 -fold, 7 -fold, 8 -fold, 9 -fold, or 10 -fold. In certain embodiments, the post-initial dose level of LIF is increased from the pre-initial dose level of LIF by at least 2 -fold, 3 -fold, 4 -fold, 5 -fold, 6 -fold, 7 -fold, 8 -fold, or 9 -fold. In certain embodiments, the post-initial dose level of LIF is increased from the pre-initial dose level of LIF by at most 3 -fold, 4 -fold, 5 -fold, 6 -fold, 7 -fold, 8 -fold, 9 -fold, or 10 -fold.

If the assay is conducted after treatment and no increase in LIF level is observed compared to a pre-treatment sample or compared to a reference level, then a subsequent dose can be administered in a higher amount or on a shorter schedule. In certain embodiments, if the amount of LIF detected in the biological sample is less than about 100, 200, 300, 400, 500, 600, 700, 800, 900 pg/mL, or less than about 1, 2, 3, 4, 5, 6, 7, 8, 9, 10, 15, 20, 25, 30, 35, 40, 45, 50, 60, 70, 80, 90, or 100 ng/mL, then a subsequent dose can be administered in an increased amount compared to a previous dose. In certain embodiments, if the amount of LIF detected in the biological sample is less than about 5, 6, 7, 8, 9, 10, 15, 20, 25, 30, 35, 40, 45, 50, 60, 70, 80, 90, 100 ng/mL, then a subsequent dose can be administered on a shorter schedule compared to a previous dose. In certain embodiments, if the amount of LIF detected in the biological sample is less than about 1, 2, 3, 4, 5, 6, 7, 8, or 9 ng/mL, then a subsequent dose can be administered in an increased amount compared to a previous dose. In certain embodiments, if the amount of LIF detected in the biological sample is less than about 1, 2, 3, 4, 5, 6, 7, 8, or 9 ng/mL, then a subsequent dose can be administered on a shorter schedule compared to a previous dose.

In certain embodiments, if the increase in LIF detected in the biological sample compared to the level observed before a previous dose, or no dose, is less than about 1.5-fold, 2-fold, 3-fold, 4-fold, 5-fold, 6-fold, 7-fold, 8-fold, 9-fold, or 10-fold, then a subsequent dose can be administered in an increased amount compared to the previous dose. In certain embodiments, if the increase in LIF detected in the biological sample compared to the level observed before a previous dose, or no dose, is less than about 1.5-fold, 2-fold, 3-fold, 4-fold, 5-fold, 6-fold, 7-fold, 8-fold, 9-fold or 10-fold, then a subsequent dose can be administered on a shorter schedule compared to the previous dose.

### Capture and Detection Antibodies

The capture antibodies of the current disclosure are useful when immobilized on a solid support, and specifically bind to an epitope or region of LIF distinct from the detecting antibody and/or the therapeutic antibody. The solid support can be a plate, column, resin, or a bead in solution. Suitable plates include well plates of the kind that can be used to assay several samples at once, such as, 96-well plates, 384-well plates, or 1,536 well plates. Common plates include polystyrene plates (e.g., medium or high-binding polystyrene). In some embodiments, the solid support is an analytical protein array, such as an antibody array, with the capture antibody arrayed. Resins or column matrices can also be used which comprise protein A, Protein G, Protein A/G, amine coupling resins, or sulfhydryl coupling resins. Beads can be agarose beads or magnetic beads of a size that allows for separation by gravity, centrifugation, or magnetic field. Beads can be used which comprise protein A, Protein G, Protein A/G, amine groups for coupling, or sulfhydryl groups for coupling. When immobilized to a solid support at least a fraction of the capture antibody is immobilized such that the antibody can bind LIF through the Fab, and produce a signal when paired with an appropriately labeled detecting antibody. The attachment of the antibody can be through a covalent interaction, such as, thiol-cross-linking, N-oxysuccinimide, maleimide, and hydrazide groups; or non-covalent interaction, such as, streptavidin-biotin coupling, passive absorption, or an affinity interaction. In certain embodiments, the solid support comprises an electrically conductive substance capable of inducing an electrochemical signal (e.g., an MSD plate; Meso Scale Diagnostics; Rockville, Md.; Cat. No. L15XA-3). The solid support can be configured such that addition of sample, detecting antibodies, and washes can be applied through the use of an automated system such as a plate washer or a fluidic device, such a syringe pump, peristaltic device, or a microfluidic device.

The detecting antibodies of the current disclosure are useful when able to bind to an epitope or region of LIF distinct from the capture antibody and/or the therapeutic antibody. The detecting antibody can be an unmodified antibody that is capable of being specifically bound by a labelled secondary antibody. The detecting antibody can be coupled to a detectable moiety, meaning that the detectable moiety is covalently coupled to the antibody, or by a small molecule affinity interaction (e.g., biotin-streptavidin). The detecting antibody can be an antibody labeled by a small molecule that is capable of being specifically bound by a labelled molecule able to bind the small molecule. For example, the detecting antibody can be coupled to biotin, which can be bound by streptavidin coupled to an appropriate detectable moiety. In some embodiments, the detecting antibody is coupled directly to a detectable moiety. The detecting antibody can be labeled with an enzymatic, luminescent, chemiluminescent, fluorescent, phosphorescent, radioactive, or nucleic acid labeling moiety. Enzymatic labeling moieties include, without limitation, horseradish peroxidase, alkaline phosphatase, beta-galactosidase, and glucose oxidase. Fluorescent labeling moieties include, without limitation, fluorescein isothiocyanate (FITC), Rhodamine, Hoechst, 4′, 6-diamidino-2-phenylindole (DAPI), sulforhodamine 101 acid chloride (Texas Red), Phycoerythrin (PE), Allophycocyanin (APC), Alexa Flour series dyes, and combinations thereof. Radioactive labeling moieties include, without limitation, ³²P, ³⁵S, ¹²⁵I, ³H, and ¹⁴C. Nucleic acid detectable moieties can be unique nucleic acid sequences (bar codes) that can be quantified by amplification or sequencing. Electrochemiluminescence detection moieties include, without limitation, Ruthenium (II) tris-bipyridine, and Ruthenium (II) tris-bipyridine-(4-methylsulfonate), Ruthenium (II) tris-bipyridine (4-methylsulfonate), conjugated via N-hydroxysuccinimide-ester to an antibody.

The antibody A4 is one antibody that can be usefully deployed in the current method of determining total LIF levels. While exemplified for use as a capture antibody, the antibody is also available for use as a detecting antibody if paired with a different capture antibody (e.g., the 7C3 of this disclosure). The A4 antibody is a rabbit monoclonal antibody. The A4 antibody does not interfere with binding of h5D8. In certain embodiments, described herein, the A4 antibody is an antibody that specifically binds LIF comprising a heavy chain variable region comprising an amino acid sequence at least about 80%, about 90%, about 95%, about 97%, about 98%, or about 99% identical to the amino acid sequence set forth in SEQ ID NO:41; and a light chain variable region comprising an amino acid sequence at least about 80%, about 90%, about 95%, about 97%, about 98%, or about 99% identical to the amino acid sequence set forth in SEQ ID NO: 42. In certain embodiments, described herein, is an antibody that specifically binds LIF comprising a heavy chain variable region comprising an amino acid sequence identical to the amino acid sequence set forth in SEQ ID NO: 41; and a light chain variable region comprising an amino acid sequence identical to the amino acid sequence set forth in SEQ ID NO: 42. The A4 antibody also encompasses the use of the CDRs of the A4 antibody, defined as detailed by any of the methods in this disclosure, engineered into other framework regions (e.g., humanized, murinized, etc.); or the variable regions mated with constant regions of other species (e.g., chimeric antibodies). Additionally, antibodies that bind the same or the similar epitope as the A4 antibody can be used. E21 is one such antibody, rat monoclonal clone JNH40O8E21 (Creative Diagnostics; Shirley, N.Y.). E21 binds a similar epitope as A4 and is compatible with the assay described herein.

Another antibody that can be usefully deployed in the current method is the 7C3 antibody. 7C3 is a mouse monoclonal antibody, clone M017C3 (BioLegend, San Diego). While exemplified for use as a detecting antibody, the antibody is also available for use as a capture antibody if paired with a different detecting antibody (e.g., the A4 of this disclosure). The 7C3 antibody also encompasses the use of the CDRs of the 7C3 antibody, defined as detailed by any of the methods in this disclosure, engineered into other framework regions (e.g., humanized, murinized, etc.); or the variable regions mated with constant regions of other species (e.g., chimeric antibodies). Additionally, antibodies that bind the same or the similar epitope as the 7C3 antibody can be used. These antibodies include those that bind human LIF in a region of LIF that interacts with the LIF receptor. One such antibody is the B7 antibody, a rabbit monoclonal antibody, set forth in SEQ ID NOs 43 (heavy chain variable region) and 44 (light chain variable region). In certain embodiments, described herein, the B7 antibody is an antibody that specifically binds LIF comprising a heavy chain variable region comprising an amino acid sequence at least about 80%, about 90%, about 95%, about 97%, about 98%, about 99%, or about 100% identical to the amino acid sequence set forth in SEQ ID NO:43; and a light chain variable region comprising an amino acid sequence at least about 80%, about 90%, about 95%, about 97%, about 98%, about 99%, or about 100% identical to the amino acid sequence set forth in SEQ ID NO: 44. Another such antibody is the D7 antibody, a rabbit monoclonal antibody, set forth in SEQ ID NOs 45 (heavy chain variable region) and 46 (light chain variable region). In certain embodiments, described herein, the D7 antibody is an antibody that specifically binds LIF comprising a heavy chain variable region comprising an amino acid sequence at least about 80%, about 90%, about 95%, about 97%, about 98%, about 99%, or about 100% identical to the amino acid sequence set forth in SEQ ID NO: 45; and a light chain variable region comprising an amino acid sequence at least about 80%, about 90%, about 95%, about 97%, about 98%, about 99%, or about 100% identical to the amino acid sequence set forth in SEQ ID NO: 46.

A desirable trait of the detecting and capture antibodies of the current disclosure is that neither antibody interferes with each other for binding LIF, and that each antibody can bind LIF whether or not a therapeutic antibody (e.g., h5D8) is bound to LIF. In certain embodiments, neither of the capture or detecting antibodies bind an epitope that overlaps with an epitope bound by h5D8. In certain embodiments, neither of the capture or detecting antibodies bind an epitope that is within 5, 10, 15, or 20 angstroms of an epitope bound by h5D8. In certain embodiments, neither of the capture or detecting antibodies bind an epitope that interacts with the LIF co-receptor gp130.

One advantage of the assay described herein is sensitivity. In healthy individuals LIF exists at a low level at steady state. In certain embodiments, the assay has a detection threshold for LIF in plasma or serum to at least about 20 pg/ml (e.g., 1 pM), 30 pg/mL, 40 pg/mL, 50 pg/mL, 60 pg/mL, 70 pg/mL, 80 pg/mL, 90 pg/mL, or 100 pg/mL. Another advantage of the assay described herein is low internal variability. In certain embodiments, the internal variability is less than about 20%, 15%, 10%, 5%, 4%, or 3%.

### Therapeutic Antibodies

The 5D8 antibody described herein was generated from rats immunized with DNA encoding human LIF. The parental rat version of the antibody is referred to as r5D8 the humanized version is referred to as h5D8.

### 5D8

The antibodies described herein were generated from rats immunized with DNA encoding human LIF. One such antibody (5D8) was cloned and sequenced and comprises CDRs (using the combination of the Kabat and IMGT CDR numbering methods) with the following amino acid sequences: a VH-CDR1 corresponding to SEQ ID NO: 1 (GFTFSHAWMH), a VH-CDR2 corresponding to SEQ ID NO: 4 (QIKAKSDDYATYYAESVKG), a VH-CDR3 corresponding to SEQ ID NO: 6 (TCWEWDLDF), a VL-CDR1 corresponding to SEQ ID NO: 9 (RSSQSLLDSDGHTYLN), a VL-CDR2 corresponding to SEQ ID NO: 11 (SVSNLES), and a VL-CDR3 corresponding to SEQ ID NO: 13 (MQATHAPPYT). This antibody has been humanized by CDR grafting and the humanized version is referred to as h5D8. The humanized variable regions of the h5D8 antibody is set forth in SEQ ID NO: 15 and SEQ ID NO: 19.

In certain embodiments, described herein, is a therapeutic antibody that specifically binds LIF comprising a VH-CDR1 at least 80% or 90% identical to that set forth in SEQ ID NO: 1 (GFTFSHAWMH), a VH-CDR2 at least 80%, 90%, or 95% identical to that set forth in SEQ ID NO: 4 (QIKAKSDDYATYYAESVKG), and a VH-CDR3 at least 80% or 90% identical to that set forth in SEQ ID NO: 6 (TCWEWDLDF). In certain embodiments, described herein, is a therapeutic antibody that specifically binds LIF comprising a VL-CDR1 at least 80% or 90% identical to that set forth in SEQ ID NO: 9 (RSSQSLLDSDGHTYLN), a VL-CDR2 at least 80% identical to that set forth in SEQ ID NO: 11 (SVSNLES), and a VL-CDR3 at least 80% or 90% identical to that set forth in SEQ ID NO: 13 (MQATHAPPYT). In certain embodiments, described herein, is a therapeutic antibody that specifically binds LIF comprising a VH-CDR1 set forth in SEQ ID NO: 1 (GFTFSHAWMH), a VH-CDR2 set forth in SEQ ID NO: 4 (QIKAKSDDYATYYAESVKG), a VH-CDR3 set forth in SEQ ID NO: 6 (TCWEWDLDF), a VL-CDR1 set forth in SEQ ID NO: 9 (RSSQSLLDSDGHTYLN), a VL-CDR2 set forth in SEQ ID NO: 11 (SVSNLES), and a VL-CDR3 set forth in SEQ ID NO: 13 (MQATHAPPYT). Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 1, 4, 6, 9, 11, and 13 by 1, 2, 3, or 4 amino acids. In certain embodiments, the antibody comprises CDRs that differ from the amino acid sequence set forth in any one of SEQ ID NOs: 1, 4, 6, 9, 11, and 13 by 1, 2, 3, or 4 amino acids and does not affect the binding affinity by greater than 10%, 20%, or 30%. In certain embodiments, antibodies that specifically bind LIF comprise one or more human heavy chain framework regions.

In certain embodiments, described herein, is a therapeutic antibody that specifically binds LIF comprising a VH-CDR1 amino acid sequence at least 80% or 90% identical to that set forth in SEQ ID NO: 1 (GFTFSHAWMH), a VH-CDR2 amino acid sequence at least 80%, 90%, or 95% identical to that set forth in SEQ ID NO: 4 (QIKAKSDDYATYYAESVKG), and a VH-CDR3 amino acid sequence at least 80% or 90% identical to that set forth in SEQ ID NO: 8 (TSWEWDLDF). In certain embodiments, described herein, is a therapeutic antibody that specifically binds LIF comprising a VL-CDR1 amino acid sequence at least 80% or 90% identical to that set forth in SEQ ID NO: 9 (RSSQSLLDSDGHTYLN), a VL-CDR2 amino acid sequence at least 80% identical to that set forth in SEQ ID NO: 11 (SVSNLES), and a VL-CDR3 amino acid sequence at least 80% or 90% identical to that set forth in SEQ ID NO: 13 (MQATHAPPYT). In certain embodiments, described herein, is a therapeutic antibody that specifically binds LIF comprising a VH-CDR1 amino acid sequence set forth in SEQ ID NO: 1 (GFTFSHAWMH), a VH-CDR2 amino acid sequence set forth in SEQ ID NO: 4 (QIKAKSDDYATYYAESVKG), a VH-CDR3 amino acid sequence set forth in SEQ ID NO: 8 (TSWEWDLDF), a VL-CDR1 amino acid sequence set forth in SEQ ID NO: 9 (RSSQSLLDSDGHTYLN), a VL-CDR2 amino acid sequence set forth in SEQ ID NO: 11 (SVSNLES), and a VL-CDR3 amino acid sequence set forth in SEQ ID NO: 13 (MQATHAPPYT). Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 1, 4, 8, 9, 11, and 13 by 1, 2, 3, or 4 amino acids. In certain embodiments, the antibody comprises CDRs that differ from the amino acid sequence set forth in any one of SEQ ID NOs: 1, 4, 8, 9, 11, and 13 by 1, 2, 3, or 4 amino acids and does not affect the binding affinity by greater than 10%, 20%, or 30%.

In certain embodiments, described herein, is a therapeutic antibody that specifically binds LIF comprising a humanized heavy chain variable region comprising an amino acid sequence at least about 80%, about 90%, about 95%, about 97%, about 98%, or about 99% identical to the amino acid sequence set forth in any one of SEQ ID NOs: 14, 15, or 17. In certain embodiments, described herein, is a therapeutic antibody that specifically binds LIF comprising a humanized heavy chain variable region comprising an amino acid sequence set forth in any one of SEQ ID NOs: 14, 15, and 17. In certain embodiments, described herein, is a therapeutic antibody that specifically binds LIF comprising a humanized light chain variable region comprising an amino acid sequence at least about 80%, about 90%, about 95%, about 97%, about 98%, or about 99% identical to the amino acid sequence set forth in any one of SEQ ID NOs: 18-21. In certain embodiments, described herein, is a therapeutic antibody that specifically binds LIF comprising a humanized light chain variable region comprising an amino acid sequence set forth in any one of SEQ ID NOs: 18-21. In certain embodiments, the antibody specifically binds human LIF.

In certain embodiments, described herein, is a therapeutic antibody that specifically binds LIF comprising a humanized heavy chain variable region comprising an amino acid sequence at least about 80%, about 90%, about 95%, about 97%, about 98%, or about 99% identical to the amino acid sequence set forth in SEQ ID NO:15; and a humanized light chain variable region comprising an amino acid sequence at least about 80%, about 90%, about 95%, about 97%, about 98%, or about 99% identical to the amino acid sequence set forth in SEQ ID NO: 19. In certain embodiments, described herein, is a therapeutic antibody that specifically binds LIF comprising a humanized heavy chain variable region comprising an amino acid sequence set forth in SEQ ID NO: 15; and a humanized light chain variable region comprising an amino acid sequence set forth in SEQ ID NO: 19.

In certain embodiments, described herein, is a therapeutic antibody that specifically binds LIF comprising a humanized heavy chain variable region comprising an amino acid sequence at least about 80%, about 90%, about 95%, about 97%, about 98%, or about 99% identical to the amino acid sequence set forth in SEQ ID NO: 38; and a humanized light chain variable region comprising an amino acid sequence at least about 80%, about 90%, about 95%, about 97%, about 98%, or about 99% identical to the amino acid sequence set forth in SEQ ID NO: 19. In certain embodiments, described herein, is a therapeutic antibody that specifically binds LIF comprising a humanized heavy chain variable region comprising an amino acid sequence set forth in SEQ ID NO: 38; and a humanized light chain variable region comprising an amino acid sequence set forth in SEQ ID NO: 19.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in any one of SEQ ID NOs: 30-33; and a humanized light chain comprising an amino acid sequence at least about 80%, about 90%, about 95%, about 97%, about 98%, or about 99% identical to the amino acid sequence set forth in any one of SEQ ID NOs: 34-37. In certain embodiments, described herein, is a therapeutic antibody that specifically binds LIF comprising a humanized heavy chain comprising an amino acid sequence set forth in any one of SEQ ID NOs: 30-33; and a humanized light chain comprising an amino acid sequence set forth in any one of SEQ ID NOs: 34-37.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 31; and a humanized light chain comprising an amino acid sequence at least about 80%, about 90%, about 95%, about 97%, about 98%, or about 99% identical to the amino acid sequence set forth in SEQ ID NO: 35. In certain embodiments, described herein, is a therapeutic antibody that specifically binds LIF comprising a humanized heavy chain comprising an amino acid sequence set forth in SEQ ID NO: 31; and a humanized light chain comprising an amino acid sequence set forth in SEQ ID NO: 35. In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO:39; and a humanized light chain comprising an amino acid sequence at least about 80%, about 90%, about 95%, about 97%, about 98%, or about 99% identical to the amino acid sequence set forth in SEQ ID NO: 35. In certain embodiments, described herein, is a therapeutic antibody that specifically binds LIF comprising a humanized heavy chain comprising an amino acid sequence set forth in SEQ ID NO: 39; and a humanized light chain comprising an amino acid sequence set forth in SEQ ID NO: 35.

In a certain embodiments, described herein, is a recombinant antibody that specifically binds Leukemia Inhibitory Factor (LIF) comprising: a heavy chain complementarity determining region 1 (VH-CDR1) comprising an amino acid sequence set forth in SEQ ID NO: 3; a heavy chain complementarity determining region 2 (VH-CDR2) comprising an amino acid sequence set forth in SEQ ID NO: 4; a heavy chain complementarity determining region 3 (VH-CDR3) comprising an amino acid sequence set forth in SEQ ID NO: 7; a light chain complementarity determining region 1 (VL-CDR1) comprising an amino acid sequence set forth in SEQ ID NO: 9; and a light chain complementarity determining region 2 (VL-CDR2) comprising an amino acid sequence set forth in SEQ ID NO: 11; and a light chain complementarity determining region 3 (VL-CDR3) comprising an amino acid sequence set forth in SEQ ID NO: 13.

In a certain embodiments, described herein, is a recombinant antibody that specifically binds Leukemia Inhibitory Factor (LIF) comprising: a heavy chain complementarity determining region 1 (VH-CDR1) comprising an amino acid sequence set forth in SEQ ID NO: 2; a heavy chain complementarity determining region 2 (VH-CDR2) comprising an amino acid sequence set forth in SEQ ID NO: 5; a heavy chain complementarity determining region 3 (VH-CDR3) comprising an amino acid sequence set forth in SEQ ID NO: 6; a light chain complementarity determining region 1 (VL-CDR1) comprising an amino acid sequence set forth in SEQ ID NO: 10; and a light chain complementarity determining region 2 (VL-CDR2) comprising an amino acid sequence set forth in SEQ ID NO: 12; and a light chain complementarity determining region 3 (VL-CDR3) comprising an amino acid sequence set forth in SEQ ID NO: 13. Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 2, 5, 6, 10, 12, and 13 by 1, 2, 3, or 4 amino acids. In certain embodiments, the antibody comprises CDRs that differ from the amino acid sequence set forth in any one of SEQ ID NOs: 2, 5, 6, 10, 12, and 13 by 1, 2, 3, or 4 amino acids and does not affect the binding affinity by greater than 10%, 20%, or 30%.

In a certain embodiments, described herein, is a recombinant antibody that specifically binds Leukemia Inhibitory Factor (LIF) comprising: a heavy chain complementarity determining region 1 (VH-CDR1) comprising an amino acid sequence set forth in SEQ ID NO: 3; a heavy chain complementarity determining region 2 (VH-CDR2) comprising an amino acid sequence set forth in SEQ ID NO: 4; a heavy chain complementarity determining region 3 (VH-CDR3) comprising an amino acid sequence set forth in SEQ ID NO: 7; a light chain complementarity determining region 1 (VL-CDR1) comprising an amino acid sequence set forth in SEQ ID NO: 9; and a light chain complementarity determining region 2 (VL-CDR2) comprising an amino acid sequence set forth in SEQ ID NO: 11; and a light chain complementarity determining region 3 (VL-CDR3) comprising an amino acid sequence set forth in SEQ ID NO: 13. Certain conservative amino acid substitutions are envisioned in the amino acid sequences of the CDRs of this disclosure. In certain embodiments, the antibody comprises CDRs that differ from the amino acid sequence set forth in any one of SEQ ID NOs: 3, 4, 7, 9, 11, and 13 by 1, 2, 3, or 4 amino acids. In certain embodiments, the antibody comprises CDRs that differ from the amino acid sequence set forth in any one of SEQ ID NOs: 3, 4, 7, 9, 11, and 13 by 1, 2, 3, or 4 amino acids and does not affect the binding affinity by greater than 10%, 20%, or 30%.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in any one of SEQ ID NOs: 22-25; and a humanized light chain comprising an amino acid sequence at least about 80%, about 90%, about 95%, about 97%, about 98%, or about 99% identical to the amino acid sequence set forth in any one of SEQ ID NOs: 26-29. In certain embodiments, described herein, is a therapeutic antibody that specifically binds LIF comprising a humanized heavy chain comprising an amino acid sequence set forth in any one of SEQ ID NOs: 22-25; and a humanized light chain comprising an amino acid sequence set forth in any one of SEQ ID NOs: 26-29.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 23; and a humanized light chain comprising an amino acid sequence at least about 80%, about 90%, about 95%, about 97%, about 98%, or about 99% identical to the amino acid sequence set forth in of SEQ ID NO: 27. In certain embodiments, described herein, is a therapeutic antibody that specifically binds LIF comprising a humanized heavy chain comprising an amino acid sequence set forth in SEQ ID NO: 23; and a humanized light chain comprising an amino acid sequence set forth in any one of SEQ ID NO: 27.

In certain embodiments, described herein, is a therapeutic antibody that specifically binds LIF comprising a humanized heavy chain comprising an amino acid sequence at least about 80%, about 90%, about 95%, about 97%, about 98%, or about 99% identical to the amino acid sequence set forth in SEQ ID NO: 39; and a humanized light chain comprising an amino acid sequence at least about 80%, about 90%, about 95%, about 97%, about 98%, or about 99% identical to the amino acid sequence set forth in of SEQ ID NO: 27. In certain embodiments, described herein, is a therapeutic antibody that specifically binds LIF comprising a humanized heavy chain comprising an amino acid sequence set forth in SEQ ID NO: 39; and a humanized light chain comprising an amino acid sequence set forth in any one of SEQ ID NO: 27.

Epitopes Bound by Therapeutically useful LIF Antibodies

Described herein is a unique epitope of human LIF that when bound inhibits LIF biological activity (e.g., STAT3 phosphorylation) and inhibits tumor growth in vivo and produces a therapeutic effect. The therapeutic antibody of the current disclosure can be a therapeutic antibody that does not comprise the CDRs of h5D8, but binds to the same or similar epitope (amino acid residues) as h5D8. A similar epitope is one that binds within the bounds of the specified epitope. The epitope described herein consists of two discontinuous stretches of amino acids (from residue 13 to residue 32 and from residue 120 to 138 of human LIF), that are present in two distinct topological domains (alpha helixes A and C) of the human LIF protein. This binding is a combination of weak (Van der Waals attraction), medium (hydrogen binding), and strong (salt bridge) interactions. In certain embodiments, a contact residue is a residue on LIF that forms a hydrogen bond with a residue on an anti-LIF antibody. In certain embodiments, a contact residue is a residue on LIF that forms a salt bridge with a residue on an anti-LIF antibody. In certain embodiments, a contact residue is a residue on LIF that results in a Van der Waals attraction with and is within at least 5, 4, or 3 angstroms of a residue on an anti-LIF antibody. The therapeutic antibody can bind this epitope, bind to less of this epitope, or overlap with this epitope and be utilized in the assay described herein.

In certain embodiments, the therapeutic antibody described herein is an isolated antibody that binds any one, two, three, four, five, six, seven, eight, nine, ten, eleven, twelve, thirteen, fourteen, fifteen, sixteen, seventeen, eighteen, nineteen, or twenty of the following residues: A13, I14, R15, H16, P17, C18, H19, N20, Q25, Q29, Q32, D120, R123, S127, N128, L130, C131, C134, S135, or H138 of SEQ ID NO: 40. In certain embodiments, described herein is an isolated antibody that binds all of the following residues: A13, I14, R15, H16, P17, C18, H19, N20, Q25, Q29, Q32, D120, R123, S127, N128, L130, C131, C134, S135, or H138 of SEQ ID NO: 40. In certain embodiments, described herein is an isolated antibody that binds all of the following residues: A13, I14, R15, H16, P17, C18, H19, N20, Q25, Q29, Q32, D120, R123, S127, N128, L130, C131, C134, S135, or H138 of SEQ ID NO: 40. In certain embodiments, the antibody only binds residues that participate with the antibody in strong or medium interactions. In certain embodiments, the antibody only binds residues that participate with the antibody in strong interactions. In a certain embodiment, the antibody interacts with helix A and C of LIF. In a certain embodiment, the antibody blocks LIF interaction with gp130.

### Therapeutic Indications

In certain embodiments, the therapeutic antibodies disclosed herein inhibit LIF signaling in cells. In certain embodiments, the IC₅₀ for biological inhibition of the antibody under serum starved conditions in U-251 cells is less than or equal to about 100, 75, 50, 40, 30, 20, 10, 5, or 1 nanomolar. In certain embodiments, the IC₅₀ for biological inhibition of the antibody under serum starved conditions in U-251 cells is less than or equal to about 900, 800, 700, 600, 500, 400, 300, 200, or 100 nanomolar.

In certain embodiments, the therapeutic antibodies disclosed herein, are useful for treating tumors and cancers that express LIF. In certain embodiments, an individual treated with the antibodies of this disclosure has been selected for treatment as having a LIF positive tumor/cancer. In certain embodiments, the tumor is LIF positive or produces elevated levels of LIF. In certain embodiments, LIF positivity is determined in comparison to a reference value or a set pathological criteria. In certain embodiments, a LIF positive tumor expresses greater than 2-fold, 3- fold, 5-fold, 10-fold, 100-fold or more LIF than a non-transformed cell from which the tumor is derived. In certain embodiments, the tumor has acquired ectopic expression of LIF. A LIF positive tumor can be determined histologically using, for example, immunohistochemistry with an anti-LIF antibody; by commonly used molecular biology methods such as, for example, mRNA quantitation by real-time PCR or RNA-seq; or protein quantitation, for example, by western blot, flow cytometry, ELISA, or a homogenous protein quantitation assays (e.g., AlphaLISA(⁹). In certain embodiments, the antibodies can be used to treat patients diagnosed with cancer. In certain embodiments, the cancer comprises one or more cancer stem cells or is one or more cancer stem cells.

In certain embodiments, the antibodies disclosed herein, are useful for treating tumors in cancers that express the LIF receptor (CD118). A LIF receptor positive tumor can be determined by histopathology or flow cytometry, and, in certain embodiments, comprises a cell that binds a LIF receptor antibody greater than 2×, 3×, 4×, 5×, 10× or more than an isotype control. In certain embodiments, the tumor has acquired ectopic expression of the LIF receptor. In a certain embodiment, the cancer is a cancer stem cell. In a certain embodiment, a LIF positive tumor or cancer can be determined by immunohistochemistry using anti-LIF an anti-LIF antibody. In a certain embodiment, a LIF positive tumor is determined by IHC analysis with a LIF Level in the top 10%, 20%, 30%, 40%, or top 50% of tumors.

In certain embodiments, the cancer comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In certain embodiments, tumors which can be treated with the antibodies of the invention comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VlPpoma, and Wilm's tumor. In certain embodiments, the tumor/cancer to be treated with one or more antibodies of the invention comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed. In a certain embodiment, the cancer is a relapsed/refractory glioblastoma, pancreatic cancer, ovarian cancer, colon cancer, prostate cancer, or lung cancer. In certain embodiments, the cancer comprises an advanced solid tumor, glioblastoma, stomach cancer, skin cancer, prostate cancer, pancreatic cancer, breast cancer, testicular cancer, thyroid cancer, head and neck cancer, liver cancer, kidney cancer, esophageal cancer, ovarian cancer, colon cancer, lung cancer, lymphoma, or soft tissue cancer. In certain embodiments, the cancer comprises non-small cell lung cancer, epithelial ovarian carcinoma, or pancreatic adenocarcinoma. In certain embodiments, the cancer comprises an advanced solid tumor. In certain embodiments, the cancer comprises appendiceal cancer, rectal cancer, metastatic mixoid liposarcoma, and paraganglioma.

### Therapeutic Methods

In certain embodiments, the therapeutic antibodies can be administered by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered on a suitable dosage schedule, for example, weekly, twice weekly, monthly, twice monthly, etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. The therapeutic antibody can be administered at a flat dose regardless of the weight or mass of the individual to whom the h5D8 antibody is administered. The h5D8 antibody can be administered at a flat dose regardless of the weight or mass of the individual to whom the therapeutic antibody is administered, provided that the individual has a mass of at least about 37.5 kilograms. A flat dose of therapeutic antibody can be administered from about 75 milligrams to about 2000 milligrams. A flat dose of therapeutic antibody can be administered from about 225 milligrams to about 2000 milligrams, from about 750 milligrams to about 2000 milligrams, from about 1125 milligrams to about 2000 milligrams, or from about 1500 milligrams to about 2000 milligrams. A flat dose of therapeutic antibody can be administered at about 75 milligrams. A flat dose of therapeutic antibody can be administered at about 225 milligrams. A flat dose of therapeutic antibody can be administered at about 750 milligrams. A flat dose of therapeutic antibody can be administered at about 1125 milligrams. A flat dose of therapeutic antibody can be administered at about 1500 milligrams. A flat dose of therapeutic antibody can be administered at about 2000 milligrams.

Other dosages of therapeutic antibody are contemplated. A flat dose of therapeutic antibody can be administered at about 50, 100, 150, 175, 200, 250, 300, 325, 350, 375, 400, 425, 450, 475, 500, 525, 550, 575, 600, 625, 650, 675, 700, 725, 775, 800, 825, 850, 875, 900, 925, 950, 975, 1000, 1025, 1050, 1075, 1100, 1150, 1175, 1200, 1225, 1250, 1275, 1300, 1325, 1350, 1375, 1400, 1425, 1450, 1475, 1525, 1550, 1575, 1600, 1625, 1650, 1675, 1700, 1725, 1750, 1775, 1800, 1825, 1850, 1875, 1900, 1925, 1950, 1975, 2025, 2050, 2075, or 2100 milligrams. Any of these doses can be administered once a week, once every two weeks, once every three weeks, or once every four weeks.

The therapeutic antibody can be administered at a dose based on the bodyweight or mass of the individual to whom the therapeutic antibody is administered. A body weight adjusted dose of therapeutic antibody can be administered from about 1 mg/kg to about 25 mg/kg. A body weight adjusted dose of therapeutic antibody can be administered from about 3 mg/kg to about 25 mg/kg, from about 10 mg/kg to about 25 mg/kg, from about 15 mg/kg to about 25 mg/kg, or from about 20 mg/kg to about 25 mg/kg. A body weight adjusted dose of h5D8 can be administered at about 1 mg/kg. A body weight adjusted dose of therapeutic antibody can be administered at about 3 mg/kg. A body weight adjusted dose of therapeutic antibody can be administered at about 10 mg/kg. A body weight adjusted dose of therapeutic antibody can be administered at about 15 mg/kg. A body weight adjusted dose of therapeutic antibody can be administered at about 20 mg/kg. A body weight adjusted dose of therapeutic antibody can be administered at about 25 mg/kg.

The assays described herein can be used to monitor target engagement in an individual receiving treatment with a therapeutic antibody. For example, if an individual is receiving a plurality of doses the total LIF level can be measured periodically to verify target engagement with a therapeutic antibody. In certain embodiments, the assay is deployed periodically throughout a course of treatment, for example weekly, once every two weeks, once every three weeks, once every four weeks, once every five weeks, once every six weeks, once every seven weeks, or once every eight weeks. If the levels of total LIF are maintained above a reference level or are elevated compared to a pre-treatment level of the individual then no treatment modification such as a shorter schedule or increased dosage amount is administered. In certain embodiments, the reference level exceeds 5, 6, 7, 8, 9, 10, 15, 20, 25, 30, 35, 40, 45, 50, 60, 70, 80, 90, or 100 ng/mL.

The assay can also be used to increase a dosage amount or shorten a dosage schedule if the total LIF level falls below a reference level or is not increased compared to a pretreatment LIF level. In certain embodiments, the reference level is 5, 6, 7, 8, 9, 10, 15, 20, 25, 30, 35, 40, 45, 50, 60, 70, 80, 90, or 100 ng/mL.

If the assay described herein returns a result that indicates the total LIF in an individual is below a certain reference level or has increased less than a certain reference amount after a previous dose, then the schedule of antibody administration can be shortened to once a week or once every two weeks, if the initial schedule was once every three weeks. If the assay described herein returns a result that indicates the total LIF in an individual is below a certain reference level or has increased less than a certain reference amount after a previous dose, then the schedule of antibody administration can be shortened to once every one, two, or three weeks, if the initial schedule was once every four weeks.

If the assay described herein returns a result that indicates the total LIF in an individual is below a certain reference level or has increased less than a certain reference amount after a previous dose, then the amount of antibody administered can be increased by about 25%, 50%, 75%, 100%, 125%, 150%, 175%, 200%, 250%, or 300% compared to a previous dose.

If the assay described herein returns a result that indicates the total LIF in an individual is below a certain reference level or has increased less than a certain reference amount after a previous dose, then the amount of antibody administration is increased to about 2000 milligrams, if the previous dose was about 1500 milligrams, about 1125, about 750 milligrams, about 225 milligrams, or about 75 milligrams.

If the assay described herein returns a result that indicates the total LIF in an individual is below a certain reference level or has increased less than a certain reference amount after a previous dose, then the amount of antibody administered is increased to about 2000 milligrams or about 1500 milligrams, if the previous dose was about 1125, about 750 milligrams, about 225 milligrams, or about 75 milligrams.

If the assay described herein returns a result that indicates the total LIF in an individual is below a certain reference level or has increased less than a certain reference amount after a previous dose, then the amount of antibody administered is increased to about 2000 milligrams, about 1500 milligrams, or about 1125 milligrams, if the previous dose was about 750 milligrams, about 225 milligrams, or about 75 milligrams.

If the assay described herein returns a result that indicates the total LIF in an individual is below a certain reference level or has increased less than a certain reference amount after a previous dose, then the amount of antibody administered is increased to about 2000 milligrams, about 1500 milligrams, or about 1125 milligrams, or about 750 milligrams, if the previous dose was about 225 milligrams, or about 75 milligrams.

If the assay described herein returns a result that indicates the total LIF in an individual is below a certain reference level or has increased less than a certain reference amount after a previous dose, then the amount of antibody administered is increased to about 2000 milligrams, about 1500 milligrams, or about 1125 milligrams, or about 750 milligrams, or about 225 milligrams, if the previous dose was about 75 milligrams.

The assay described herein can also be used to select a patient for treatment with an anti-LIF therapeutic antibody. In certain embodiments, an individual that has not been treated with an anti-LIF therapeutic antibody can be treated with an anti-LIF therapeutic antibody if a total LIF level in a biological sample exceeds about 100, 200, 300, 400, 500, 600, 700, 800, 900 pg/mL, or about 1, 2, 3, 4, 5, 6, 7, 8, 9, 10, 15, 20, 25, 30, 35, 40, 45, 50, 60, 70, 80, 90, or 100 ng/mL. In certain embodiments, the biological sample is blood, serum, or plasma. In certain embodiments, the anti-LIF therapeutic antibody is h5D8.

Any of the doses detailed herein can be administered i.v. over a time period of at least about 60 minutes; however, this period can vary somewhat based upon conditions relevant to each individual administration.

### Pharmaceutically Acceptable Excipients, Carriers and Diluents

In certain embodiments, the antibodies of the current disclosure are administered suspended in a sterile solution. In certain embodiments, the solution comprises a physiologically appropriate salt concentration (e.g., NaCl). In certain embodiments, the solution comprises between about 0.6% and 1.2% NaCl. In certain embodiments, the solution comprises between about 0.7% and 1.1% NaCl. In certain embodiments, the solution comprises between about 0.8% and 1.0% NaCl. In certain embodiments, a highly concentrated stock solution of antibody may be diluted in about 0.9% NaCl. In certain embodiments, the solution comprises about 0.9% NaCl. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20),polysorbate and poloxamer 188; polyol/disaccharide/polysaccharides, for example, glucose, dextrose, mannose, mannitol, sorbitol, sucrose, trehalose, and dextran 40; amino acids, for example, histidine, glycine or arginine; antioxidants, for example, ascorbic acid, methionine; and chelating agents, for example, EGTA or EGTA. In certain embodiments, the antibodies of the current disclosure are shipped/stored lyophilized and reconstituted before administration. In certain embodiments, lyophilized antibody formulations comprise a bulking agent such as, mannitol, sorbitol, sucrose, trehalose, and dextran 40. In a certain embodiment, anti-LIF antibodies of this disclosure can be shipped and stored as a concentrated stock solution to be diluted at the treatment site of use. In certain embodiments, the stock solution comprises about 25mM histidine, about 6% sucrose, about 0.01% polysorbate, and about 20mg/mL of anti-LIF antibody. In certain embodiments, the pH of the solution is about 6.0. In certain embodiments, the form administered to an individual is an aqueous solution comprising about 25mM histidine, about 6% sucrose, about 0.01% polysorbate 80, and about 20mg/mL of h5D8 antibody. In certain embodiments, the pH of the solution is about 6.0.

### Embodiments

The following embodiments are further specific examples of aspects of the current disclosure. 1. A method of treating an individual with cancer comprising: (a) administering to the individual an initial dose of an antibody that binds Leukemia Inhibitory Factor (LIF); (b) determining a post-initial dose level of Leukemia Inhibitory Factor (LIF) in a sample from the individual with cancer. 2. The method according to embodiment 1, further comprising administering a subsequent dose of the antibody that binds Leukemia Inhibitory Factor (LIF). 3. The method according to embodiment 1, wherein determining a post initial dose level of LIF is performed by a method according to any one of embodiments 1 to 2. 4. The method of embodiment 1, wherein the LIF therapeutic antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and (f) an immunoglobulin light chain complementarity determining region 3 (VL-CDR3) comprising the amino acid sequence set forth in SEQ ID NO: 13. 5. The method of embodiment 1, wherein the LIF therapeutic antibody comprises: (a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and (b) an immunoglobulin light chain variable region (VL) sequence with an amino acid sequence at least about 80%, 90%, 95%, 97%, 98%, or 99% identical to the amino acid sequence set forth in any one of SEQ ID NOs: 18-21. 6. The method of embodiment 1, wherein the LIF therapeutic antibody comprises: (a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and (b) an immunoglobulin light chain sequence with an amino acid sequence at least about 80%, 90%, 95%, 97%, 98%, or 99% identical to the amino acid sequence set forth in any one of SEQ ID NOs: 34-37. 7. The method of any one of embodiments 2 to 6, wherein the post-initial dose level of LIF is not increased compared to a pre-initial dose level of LIF in the individual, and wherein the subsequent dose is administered at an increased amount compared to the initial dose. 8. The method of any one of embodiments 2 to 6, wherein the post-initial dose level of LIF is increased by 2-fold or less compared to a pre-initial dose level of LIF in the individual, and wherein the subsequent dose is administered at an increased amount compared to the initial dose. 9. The method of any one of embodiments 2 to 6, wherein the post-initial dose level of LIF is increased by 2-fold or less compared to a pre-initial dose level of LIF in the individual, and wherein the subsequent dose is administered at an increased amount compared to the initial dose. 10. The method of any one of embodiments 2 to 6, wherein the post- initial dose level of LIF is not increased compared to a pre-initial dose level of LIF in the individual, and wherein the subsequent dose is administered at an earlier point in a treatment schedule. 11. The method of any one of embodiments 2 to 6, wherein the post- initial dose level of LIF is increased by 2-fold or less compared to a pre-initial dose level of LIF in the individual, and wherein the subsequent dose is administered at an earlier point in a treatment schedule. 12. The method of any one of embodiments 2 to 6, wherein the post- initial dose level of LIF is increased by 2-fold or less compared to a pre-initial dose level of LIF in the individual, and wherein the subsequent dose is administered at an earlier point in a treatment schedule. 13. The method of any one of embodiments 1 to 12, wherein the initial dose is a first dose of the antibody that binds Leukemia Inhibitory Factor (LIF). 14. The method of any one of embodiments 1 to 12, wherein the initial dose is any dose in a plurality of doses of the antibody that binds Leukemia Inhibitory Factor (LIF). 15. A method of treating an individual with cancer comprising: (a) administering to the individual an initial dose comprising an antibody that binds Leukemia Inhibitory Factor (LIF); (b) receiving a post-initial dose level of Leukemia Inhibitory Factor (LIF) in a sample from the individual with cancer. 16. The method of embodiment 15, further comprising administering a subsequent dose of the antibody that binds Leukemia Inhibitory Factor (LIF). 17. The method of embodiment 15, wherein the LIF therapeutic antibody comprises: (a) an immunoglobulin heavy chain complementarity determining region 1 (VH-CDR1) comprising the amino acid sequence set forth in any one of SEQ ID NOs: 1-3; (b) an immunoglobulin heavy chain complementarity determining region 2 (VH-CDR2) comprising the amino acid sequence set forth in any one of SEQ ID NOs: 4 or 5; (c) an immunoglobulin heavy chain complementarity determining region 3 (VH-CDR3) comprising the amino acid sequence set forth in any one of SEQ ID NOs: 6-8; (d) an immunoglobulin light chain complementarity determining region 1 (VL-CDR1) comprising the amino acid sequence set forth in any one of SEQ ID NOs: 9 or 10; (e) an immunoglobulin light chain complementarity determining region 2 (VL-CDR2) comprising the amino acid sequence set forth in any one of SEQ ID NOs: 11 or 12; and f an immunoglobulin light chain complementarity determining region 3 (VL-CDR3) comprising the amino acid sequence set forth in SEQ ID NO: 13. 18. The method of embodiment 15, wherein the LIF therapeutic antibody comprises: (a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and (b) an immunoglobulin light chain variable region (VL) sequence with an amino acid sequence at least about 80%, 90%, 95%, 97%, 98%, or 99% identical to the amino acid sequence set forth in any one of SEQ ID NOs: 18-21. 19. The method of embodiment 15, wherein the LIF therapeutic antibody comprises: (a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and (b) an immunoglobulin light chain sequence with an amino acid sequence at least about 80%, 90%, 95%, 97%, 98%, or 99% identical to the amino acid sequence set forth in any one of SEQ ID NOs: 34-37. 20. The method of any one of embodiments 16 to 19, wherein the post-initial dose level of LIF is not increased compared to a pre-treatment level of LIF in the individual, and wherein the subsequent dose is administered at an increased amount compared to the initial dose. 21. The method of any one of embodiments 16 to 19, wherein the post- initial dose level of LIF is increased by 2-fold or less compared to a pre-initial dose level of LIF in the individual, and wherein the subsequent dose is administered at an increased amount compared to the initial dose. 22. The method of any one of embodiments 16 to 19, wherein the post-initial dose level of LIF is detectable and is less than 2 nanograms per milliliter, and wherein the subsequent dose is administered at an increased amount compared to the initial dose. 23. The method of any one of embodiments 16 to 19, wherein the post-initial dose level of LIF is not increased compared to a pre-treatment level of LIF in the individual, and wherein the subsequent dose is administered at an earlier point in a treatment schedule. 24. The method of any one of embodiments 16 to 19, wherein the post-initial dose level of LIF is increased by 2-fold or less compared to a pre- initial dose level of LIF in the individual, and wherein the subsequent dose is administered at an earlier point in a treatment schedule. 25. The method of any one of embodiments 16 to 19, wherein the post-initial dose level of LIF is detectable and is less than 2 nanograms per milliliter, and wherein the subsequent dose is administered at an earlier point in a treatment schedule. 26. The method of any one of embodiments 15 to 25, wherein the initial dose is a first dose of the antibody that binds Leukemia Inhibitory Factor (LIF). 27. The method of any one of embodiments 15 to 25, wherein the initial dose is any dose in a plurality of doses of the antibody that binds Leukemia Inhibitory Factor (LIF). 28. The use of a LIF complex comprising: LIF, a LIF capture antibody that specifically binds to LIF, a LIF detecting antibody that specifically binds to LIF, and optionally a LIF therapeutic antibody that specifically binds LIF, wherein the LIF detecting or the LIF capture antibody comprises A4 or a LIF binding fragment thereof, wherein the use is an in vitro assay to quantify LIF. 29. The LIF complex of embodiment 28, wherein the complex is in a fluid. 30. The complex of embodiment 28, wherein the assay has internal variability of less than 20%. 31. A method of quantifying Leukemia Inhibitory Factor (LIF) in a sample from an individual comprising LIF comprising: contacting the sample comprising LIF to a capture antibody that specifically binds to LIF; contacting the sample comprising LIF to a detecting antibody that specifically binds LIF; detecting the LIF in the sample that is bound to the capture antibody and the detecting antibody; wherein the LIF detecting or the LIF capture antibody comprises A4 or a LIF binding fragment thereof, wherein the method is performed in vitro. 32. The method of embodiment 31, wherein the LIF is human LIF. 33. The method of embodiment 31, wherein the LIF is detectable at a level of 1 nanogram per milliliter. 34. The method of embodiment 31, wherein the assay has internal variability of less than 20%. 35. The method of embodiment 31, wherein the individual is a human individual. 36. The method of embodiment 31, further comprising quantifying the LIF in the sample. 37. The method of embodiment 36, wherein the sample comprising LIF is in a fluid. 38. The method of embodiment 31, further comprising transmitting a report comprising information on a quantity of LIF in the sample. 39. A Leukemia Inhibitory Factor (LIF) binding antibody or fragment thereof, wherein the LIF binding antibody or fragment thereof comprises: an immunoglobulin heavy chain variable region with an amino acid sequence at least about 90% identical to the amino acid sequence set forth in SEQ ID NO: 41; and an immunoglobulin light chain variable region with an amino acid sequence at least about 90% identical to the amino acid sequence set forth in SEQ ID NO: 42. 40. Use of the LIF binding antibody of embodiment 39, in an in vitro assay to quantify LIF.

### EXAMPLES

The following illustrative examples are representative of embodiments of the compositions and methods described herein and are not meant to be limiting in any way.

### Example 1

### Generation of Rat Antibodies Specific for LIF

A cDNA encoding amino acids 23-202 of human LIF was cloned into expression plasmids (Aldevron GmbH, Freiburg, Germany). Groups of laboratory rats (Wistar) were immunized by intradermal application of DNA-coated gold-particles using a hand-held device for particle-bombardment (“gene gun”). Cell surface expression on transiently transfected HEK cells was confirmed with anti-tag antibodies recognizing a tag added to the N-terminus of the LIF protein. Serum samples were collected after a series of immunizations and tested in flow cytometry on HEK cells transiently transfected with the aforementioned expression plasmids. Antibody-producing cells were isolated and fused with mouse myeloma cells (Ag8) according to standard procedures. Hybridomas producing antibodies specific for LIF were identified by screening in a flow cytometry assay as described above. Cell pellets of positive hybridoma cells were prepared using an RNA protection agent (RNAlater, cat. #AM7020 by ThermoFisher Scientific) and further processed for sequencing of the variable domains of the antibodies.

### Example 2

### Generation of Mouse Antibodies Specific for LIF

A cDNA encoding amino acids 23-202 of human LIF was cloned into expression plasmids (Aldevron GmbH, Freiburg, Germany). Groups of laboratory mice (NMRI) were immunized by intradermal application of DNA-coated gold-particles using a hand-held device for particle-bombardment (“gene gun”). Cell surface expression on transiently transfected HEK cells was confirmed with anti-tag antibodies recognizing a tag added to the N-terminus of the LIF protein. Serum samples were collected after a series of immunizations and tested in flow cytometry on HEK cells transiently transfected with the aforementioned expression plasmids. Antibody-producing cells were isolated and fused with mouse myeloma cells (Ag8) according to standard procedures. Hybridomas producing antibodies specific for LIF were identified by screening in a flow cytometry assay as described above. Cell pellets of positive hybridoma cells were prepared using an RNA protection agent (RNAlater, cat. #AM7020 by ThermoFisher Scientific) and further processed for sequencing of the variable domains of the antibodies.

### Example 3

### Humanization of Rat Antibodies Specific for LIF

One clone from the rat immunization (5D8) was chosen for subsequent humanization. Humanization was conducted using standard CDR grafting methods. The heavy chain and light chain regions were cloned from the 5D8 hybridoma using standard molecular cloning techniques and sequenced by the Sanger method. A BLAST search was then conducted against human heavy chain and light chain variable sequences and 4 sequences from each were chosen as acceptor frameworks for humanization. These acceptor frameworks were deimmunized to remove T cell response epitopes. The heavy chain and light chain CDR1, CDR2 and CDR3 of 5D8 were cloned into the 4 different heavy chain acceptor frameworks (H1 to H4), and 4 different light chain frameworks (L1 to L4). Then all 16 different antibodies were tested for: expression in CHO-S cells (Selexis); inhibition of LIF-induced STAT3 phosphorylation; and binding affinity by Surface Plasmon Resonance (SPR). These experiments are summarized in

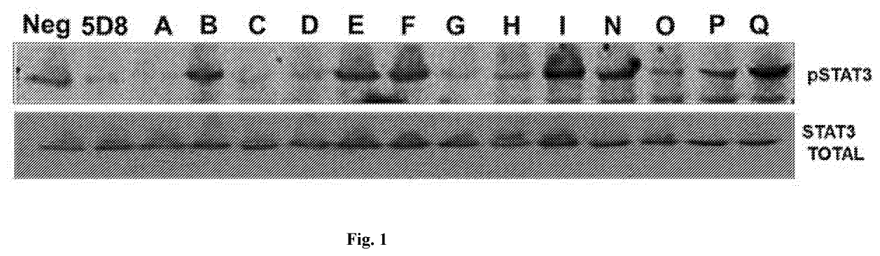

TABLE 1
Summary of 5D8 humanization

| Heavy chain | Inhibition of | Affinity by | |
|---|---|---|---|
| light chain | LIF-induced | SPR K_{D1} | Expression |
| combination | pSTAT3 from FIG. 1 | (pM) | (ug/mL) |
| H0L0 | +++ | 133 ± 46 | 393 |
| H1L1 | − | N/A | 627 |
| H1L2 | +++ | 55 ± 23 | 260 |
| H1L3 | +++ | 54 ± 31 | 70 |
| H1L4 | − | N/A | 560 |
| H2L1 | − | N/A | 369 |
| H2L2 | +++ | 52 ± 22 | 392 |
| H2L3 | ++ | 136 ± 19 | 185 |
| H2L4 | − | N/A | 78 |
| H3L1 | N/A | N/A | No expression |
| H3L2 | N/A | N/A | No expression |
| H3L3 | N/A | N/A | No expression |
| H3L4 | N/A | N/A | No expression |
| H4L1 | − | N/A | 259 |
| H4L2 | ++ | 913 ± 308 | 308 |
| H4L3 | + | | 252 |
| H4L4 | − | N/A | 186 |

N/A = Not attempted;
H0L0 = chimeric antibody with full rat heavy and light chain variable regions

The expression performance of the transfected cells was compared in Erlenmeyer flasks (seeding 3×10⁵ cells/mL, 200 mL culture volume) within fed-batch cultivation after 10 days of cell culture. At this point cells were harvested and the secreted antibody purified using a Protein A column and then quantitated. All humanized antibodies expressed except those using the H3 heavy chain. The H2 and L2 variable regions performed well compared to other variable regions (SEQ ID NO: 15 and SEQ ID NO: 19).

Inhibition of LIF-induced STAT3 phosphorylation at tyrosine 705 was determined by western blot. U251 glioma cells were plated in 6-well plates at a density of 100,000 cells/well. Cells were cultured in complete medium for 24 hours before any treatment and after that, cells were serum starved for 8 hours. After that, cells with the indicated antibodies over night at a concentration of 10 μg/ml. After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fat dried milk—TBST and incubated with the primary antibody overnight (p-STAT3, catalog #9145, Cell Signaling or STAT3, catalog #9132, Cell Signaling) or 30 minutes (β-actin-peroxidase, catalog #A3854, Sigma-Aldrich). Membranes were then washed with TBST, incubated with secondary and washed again. Proteins were detected by chemiluminescence (SuperSignal Substrate, catalog #34076, Thermo Fisher Scientific). These results are shown in FIG. 1. The darker the pSTAT3 band the less inhibition is present. Inhibition was high in lanes labeled 5D8 (non-humanized rat), A(H0L0), C (H1L2), D (H1L3), and G (H2L2); inhibition was moderate in H (H2L3), O (H4L2), and P (H4L3); inhibition was absent in B (H1L1), E (H1L4), F (H2L1), I (H2L4), N (H4L1) and Q (H4L4).

Antibodies that exhibited inhibition of LIF-induced STAT3 phosphorylation were then analyzed by SPR to determine binding affinity. Briefly, binding of the A(H0L0), C (H1L2), D (H1L3), and G (H2L2), H (H2L3) and O (H4L2) humanized antibodies to amine coupled hLIF was observed using a BiacoreTM 2002 Instrument. Kinetic constants and affinities were determined by mathematical sensorgram fitting (Langmuir interaction model [A +B =AB]) of all sensorgrams generated on all sensor chip surfaces at six ligand concentrations. The best fitted curves (minimal Chi2) of each concentration were used for calculation of kinetic constants and affinities. See Table 1.

Since the experimental setup used bivalent antibodies as analytes, best fitted sensorgrams, were also analyzed on basis of a bivalent analyte fitting model [A+B=AB; AB+B=AB2] in order to obtain a more detailed insight into the target binding mechanism of the humanized antibodies. Kinetic sensorgram analysis using a bivalent fitting model [A+B=AB; AB+B=AB2] confirmed the relative affinity ranking of the mAb samples.

The humanized 5D8 comprising H2 and L2 was selected for more in-depth analysis due to its high binding affinity and high yield from batch culture.

### Example 4

### Humanization of clone 5D8 improves binding to LIF

We selected the H2L2 clone (h5D8) for further analysis and compared binding by SPR to the parental rat 5D8 (r5D8) and a mouse clone 1B2. The 1B2 antibody is a previously disclosed mouse anti-LIF antibody previously deposited at the Deutsche Sammlung von Mikroorganismen and Zellkulturen GmbH (DSM ACC3054) and was included for comparison purposes. Recombinant human LI, purified from E. coli and HEK-293 cells, respectively, were used as ligands. The LIF from human or E. coli sources was covalently coupled to the surface of Biacore optical sensor chips using amine coupling chemistry, and binding affinities were calculated from the kinetic constants.

### Materials and Methods

Human LIF from E. coli was obtained from Millipore, reference LIF 1010; human LIF from HEK-293 cells was obtained from ACRO Biosystems, reference LIF-H521b. LIF was coupled to the sensor chips using the Biacore Amine Coupling Kit (BR-1000-50; GE-Healthcare, Uppsala). Samples were run on a Biacore™ 2002 Instrument using CM5 optical sensor chips (BR-1000-12; GE-Healthcare, Uppsala). Biacore HBS-EP buffer was used during the machine runs (BR-1001-88; GE-Healthcare, Uppsala). Kinetic analysis of binding sensorgrams was performed using BlAevaluation 4.1 software. Kinetic constants and affinities were determined by mathematical sensorgram fitting (Langmuir interaction model [A+B=AB]) of all sensorgrams generated on all sensor chip surfaces at increasing analyte concentrations. Sensorgrams were also analyzed on the basis of a bivalent analyte sensorgram fitting model [A+B=AB; AB+B=AB₂], including component analysis, in order to generate an estimate on the bivalent contribution to the determined Langmuir antibody—target affinities (e.g., avidity contribution). The best fitted curves (minimal Chi^{t}) of each concentration were used for calculation of kinetic constants and affinities. Summaries of these affinity experiments are shown in Table 2 (human LIF made in E. coli) and Table 3 (human LIF made in HEK 293 cells).

TABLE 2
Improved binding of 5D8 after humanization

| | K_{D} [pM] | |
|---|---|---|
| | Langmuir 1:1 | Bivalent analyte |
| hLIF (E. coli) | sensorgram fitting | fitting |
| Mouse 1B2 | 400 ± 210 | 1500 ± 200 |
| r5D8 (Rat) | 130 ± 30 | 780 ± 130 |
| h5D8 (humanized) | 26 ± 14 | 82 ± 25 |

TABLE 3
Improved binding of 5D8 after humanization

| | K_{D} [pM] | |
|---|---|---|
| | Langmuir 1:1 | Bivalent analyte |
| hLIF (HEK 293) | sensorgram fitting | fitting |
| Mouse 1B2 | 320 ± 150 | 3900 ± 900 |
| r5D8 (rat) | 135 ± 100 | 410 ± 360 |
| h5D8 (humanized) | 13 ± 6 | 63 ± 30 |

The Langmuir 1:1 sensorgram fitting model from this set of experiments indicates that the humanized 5D8 (h5D8) antibody bound with ˜10-25 times higher affinity to human LIF than mouse 1B2 and r5D8.

Next, the h5D8 antibody was tested against LIF of multiple species by SPR. h5D8 SPR binding kinetics were performed for recombinant LIF analytes derived from different species and expression systems: human LIF (E. coli, HEK293 cells); mouse LIF (E. coli, CHO cells); rat LIF (E. coli); cynomolgus monkey LIF (yeast, HEK293 cells).

### Materials and Methods

The h5D8 antibody was immobilized to the sensor chip surface by non-covalent, Fc specific capturing. Recombinant, Ig(Fc) specific S. aureus Protein A/G was used as capturing agent, allowing sterically uniform and flexible presentation of the anti-LIF antibody to the LIF analytes. Sources of the LIF analytes are as follows: Human LIF (from E. coli; Millipore reference LIF 1050); Human LIF (from HEK cells ACRO Biosystems LIF-H521); Mouse LIF (E. coli; Millipore Cat. No NF-LIF2010); Mouse LIF (from CHO cells; Reprokine Catalog # RCP09056); Monkey LIF (yeast Kingfisher Biotech Catalog # RP1074Y); Monkey LIF produced in HEK-293 cell. Overall h5D8 exhibited binding to LIF from several species. A summary of this affinity experiment is shown in Table 4.

TABLE 4
Broad species reactivity of humanized 5D8

| | Langmuir 1:1 sensorgram fitting | | |
|---|---|---|---|
| | mean Kₐ | mean K_{d} (1/S) | mean K_{D} |
| Analyte | (1/Ms)[10⁵] | [10⁻⁵] | [pM] |
| Human LIF (E.coli) | 8.5 ± 0.7 | 7.2 ± 0.7 | 86 ± 9 |
| Human LIF (HEK-293) | 5.5 ± 0.02 | 3.1 ± 0.7 | 56 ± 13 |
| Mouse LIF (E.coli) | 21.4 ± 3.7 | 5.7 ± 1.0 | 27 ± 6 |
| Mouse LIF(CHO cells) | 6.5 ± 0.7 | 1.1 ± 0.3 | 17 ± 4 |
| Cyno Monkey LIF (yeast) | 6.3 ± 0.8 | 5.4 ± 0.7 | 89 ± 10 |
| Cyno Monkey LIF (HEK-293) | 2.4 ± 0.2 | 3.3 ± 0.3 | 134 ± 6 |

### Example 5

### Humanized Clone 5D8 Inhibits LIF-Induced Phosphorylation of STAT3 In vitro

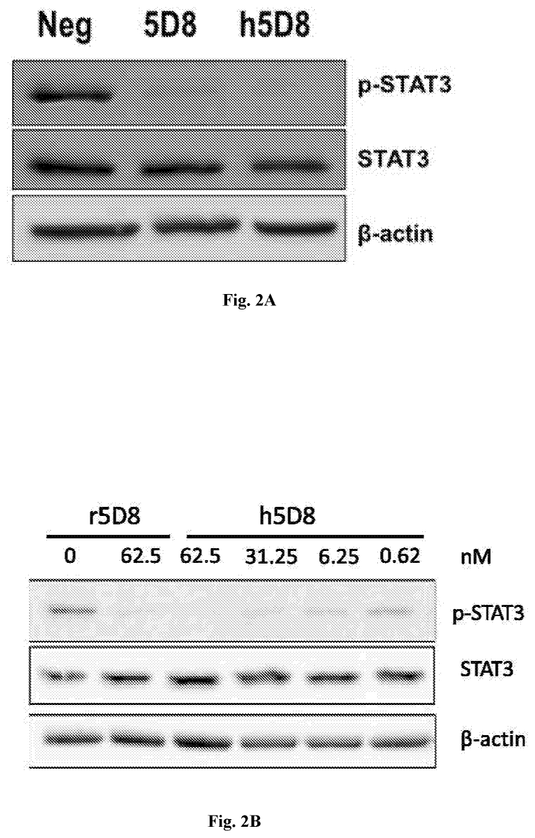

To determine the biological activity of h5D8, the humanized and parental versions were tested in a cell culture model of LIF activation. FIG. 2A shows that the humanized clone exhibited increased inhibition of STAT3 phosphorylation (Tyr 705) when a glioma cell line was incubated with human LIF. FIG. 2B shows an experiment with the same set up of FIG. 2A repeated with different dilutions of the h5D8 antibody.

### Methods

U251 glioma cells were plated in 6-well plates at a density of 150,000 cells/well. Cells were cultured in complete medium for 24 hours before any treatment. After that, cells were treated over night or not (control cells) with r5D8 anti-LIF antibody or h5D8 anti-LIF antibody at a concentration of 10 pg/ml.

After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fatty milk - TBST and incubated with the primary antibody overnight (p-STAT3, catalog #9145, Cell Signaling or STAT3, catalog #9132, Cell Signaling) or 30 minutes (β-actin-peroxidase, catalog #A3854, Sigma-Aldrich). Membranes were then washed with TBST, incubated with secondary antibody if necessary, and washed again. Proteins were detected by chemiluminescence (SuperSignal Substrate, catalog #34076, Thermo Fisher Scientific).

### Example 6

### IC₅₀ Value of h5D8 Antibody Treatment on Endogenous Levels of LIF in U-251 Cells

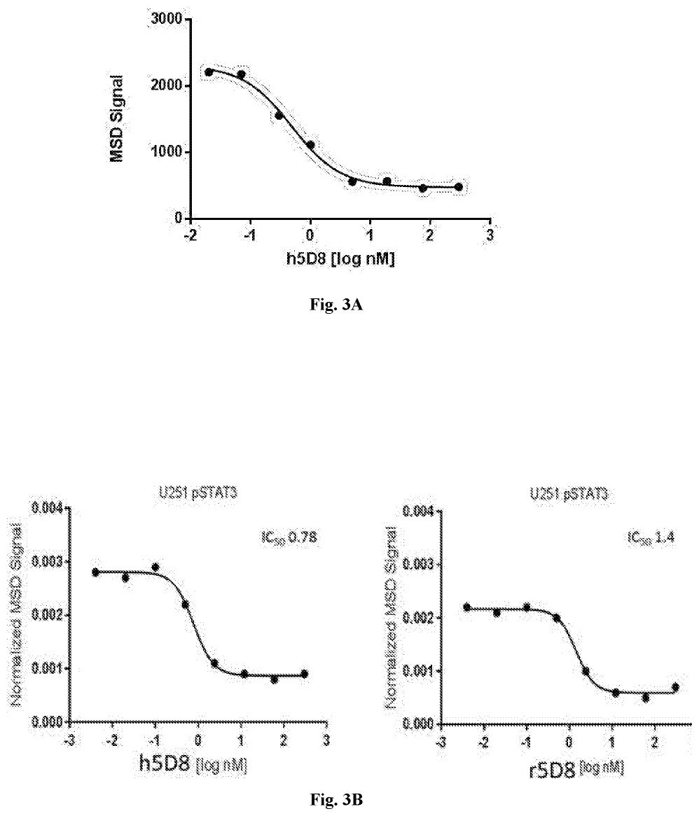

We also determined an IC₅₀ of as low as 490 picomolar (FIG. 3A) for biological inhibition for h5D8 under serum starved conditions in U-251 cells. See representative results FIG. 3A and 3B and Table 5.

TABLE 5

| Cell | Cell | | | | | | | JAK |
|---|---|---|---|---|---|---|---|---|
| Line | Line | Treat- | | | | | | inhi- |
| Tissue | Name | ment | | | | | IC₉₀ | bition |
| Endogenous | | | IC₅₀ (nM) | | | | (nM) | (%) |
| LIF Condition | | | n = 1 | n = 2 | Mean | SD | Mean | Mean |
| GBM | U251 | h5D8 | 0.78 | 0.54 | 0.66 | 0.12 | 4.1 | 84% |
| | | r5D8 | 1.6 | 1.5 | 1.4 | 0.15 | 8.5 | 86% |
| | | | 1.2 | 1.4 | | | | |

### Methods

The U-251 cells were seeded at 600,000 cells per 6cm plate (per condition). Cells were treated with h5D8 in corresponding concentration (titration) overnight at 37° C., under serum starvation (0.1% FBS). As a positive control for pSTAT3, recombinant LIF (R&D #7734-LF/CF) was used to stimulate the cells at 1.79 nM for 10min at 37° C. As a negative control of pSTAT3, the JAK I inhibitor (Calbiochem #420099) was used at luM for 30min at 37° C. Cells were then harvested on ice for lysates following the Meso Scale Discovery Multi-Spot Assay System Total STAT3 (Cat# K150SND-2) and Phospho-STAT3 (Tyr705) (Cat# K150SVD-2) kits' protocol, to measure protein levels detectable by the MSD Meso Sector S600.

### Example 7

### Additonal Antibodies that Specifically Bind to Human LIF

Other rat antibody clones (10G7 and 6B5) that specifically bind human LIF were identified and a summary of their binding characteristics are shown below in Table 6, clone 1B2 served as a comparison.

### Methods

Kinetic real time binding analysis was performed for anti-LIF mAbs 1B2, 10G7 and 6B5, immobilized on the surface of CM5 optical sensor chips, applying recombinant LIF target proteins [human LIF (E. coli); Millipore Cat. No. LIF 1010 and human LIF (HEK293 cells); ACRO Biosystems Cat. No. LIF-H521b] as analytes.

Kinetic constants and affinities were obtained by mathematical sensorgram fitting using a Langmuir 1:1 binding model applying global (simultaneous fitting of sensorgram sets) as well as single curve fitting algorithms. Plausibility of global fits was assessed by k_{obs} analysis.

TABLE 6
Affinity measurements of additional anti-LIF antibodies

| | | Langmuir 1:1 sensorgram fitting | | |
|---|---|---|---|---|
| | | mean Kₐ | mean K_{d} | mean K_{D} |
| Analyte | clone | (1/Ms) | (1/S) | [nM] |
| Human LIF | 1B2 | 1.1 ± 0.4E5 | 1.1 ± 0.3E−3 | 9.7 ± 1.4 |
| (E.coli) | | | | |
| Human LIF | 1B2 | 2.0 ± 0.04E6 | 1.4 ± 0.2E−3 | 0.7 ± 0.03 |
| (HEK-293) | | | | |
| Human LIF | 10G7 | 7.9 ± 5.8E4 | 6.0 ± 2.3E−4 | 12.6 ± 9.5 |
| (E.coli) | | | | |
| Human LIF | 10G7 | 3.6 ± 1.75E5 | 3.1 ± 0.5E−4 | 1.1 ± 0.6 |
| (HEK-293) | | | | |
| Human LIF | 6B5 | N/A | N/A | N/A |
| (E.coli) | | | | |
| Human LIF | 6B5 | 3.6 ± 1.7E5 | 3.1 ± 0.5E−4 | 62 ± 6 |
| (HEK-293) | | | | |

### Example 8

### Additional anti LIF Antibodies Inhibit LIF-Induced Phosphorylation of STAT3In Vitro

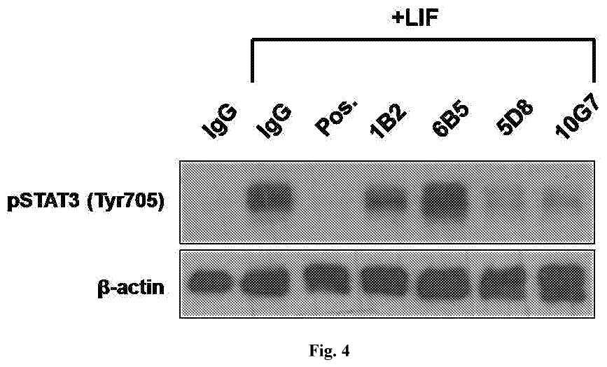

Additional clones were tested for their ability to inhibit LIF-induced phosphorylation of STAT3 in cell culture. As shown in FIG. 4 clones 10G7 and the previously detailed r5D8 exhibited high inhibition of LIF-induced STAT3 phosphorylation, compared to the 1B2 clone. Anti-LIF polyclonal anti-sera (pos.) was included as a positive control While 6B5 exhibited no inhibition, this may be explained by a possible lack of 6B5 binding to non-glycosylated LIF which was used in this experiment.

### Methods

Patient derived glioma cells were plated in 6-well plates at a density of 150,000 cells/well. Cells were cultured in GBM medium that consisted of Neurobasal medium (Life Technologies) supplemented with B27 (Life Technologies), penicillin/streptomycin and growth factors (20 ng/ml EGF and 20 ng/ml FGF-2 [PeproTech]) for 24 hours before any treatment. The following day, cells were treated or not with recombinant LIF produced in E. coli or a mix of recombinant LIF plus the indicated antibodies for 15 minutes (final concentration of 10 pg/ml for the antibodies and 20 ng/ml of recombinant LIF). After treatment, proteins were obtained in radio-immunoprecipitation assay (RIPA) lysis buffer containing phosphatase and protease inhibitors, quantified (BCA-protein assay, Thermo Fisher Scientific) and used in western blot. For western blot, membranes were blocked for 1 hour in 5% non-fatty milk—TBST and incubated with the primary antibody overnight (p-STAT3, catalog #9145, Cell Signaling) or 30 minutes (β-actin-peroxidase, catalog #A3854, Sigma-Aldrich). Membranes were then washed with TBST, incubated with secondary antibody if necessary, and washed again. Proteins were detected by chemiluminescence (SuperSignal Substrate, catalog #34076, Thermo Fisher Scientific).

### Example 9

### LIF is Highly Overexpressed Across Multiple Tumor Types

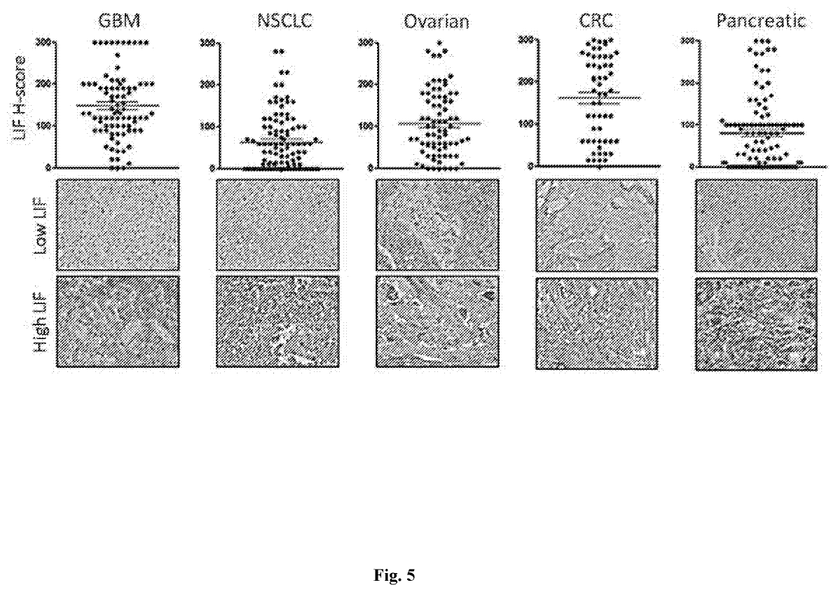

Immunohistochemistry was conducted on multiple human tumor types to determine the degree of LIF expression. As shown in FIG. 5 LIF is highly expressed in glioblastoma multiforme (GBM), non-small cell lung cancer (NSCLC), ovarian cancer, and colorectal cancer (CRC).

### Example 10

### Humanized Clone h5D8 Inhibits Tumor Growth in a Mouse Model of Non-Small cell Lung Carcinoma

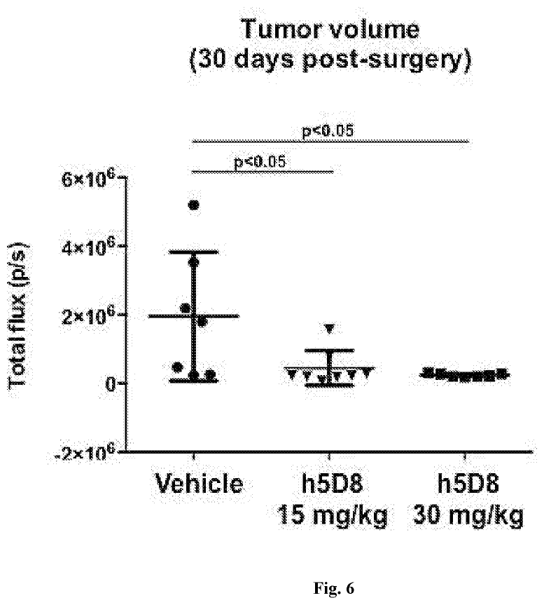

To determine the ability of the humanized 5D8 clone to inhibit a LIF positive cancer in vivo this antibody was tested in a mouse model of non-small cell lung carcinoma (NSCLC). FIG. 6 shows reduced tumor growth in mice treated with this antibody compared to a vehicle negative control.

### Methods

The murine non-small cell lung cancer (NSCLC) cell line KLN205 with high LIF levels was stably infected with lentivirus expressing the firefly luciferase gene for in vivo bioluminescence monitoring. To develop the mouse model, 5×10⁵KLN205 non-small cell lung cancer (NSCLC) cells were orthotopically implanted into the left lung of 8-week-old immunocompetent syngeneic DBA/2 mice by intercostal puncture. Mice were treated with a control vehicle or with 15 mg/kg or 30 mg/kg of the h5D8 antibody intraperitoneally twice a week and tumor growth was monitored by bioluminescence. For the bioluminescence imaging, mice received an intraperitoneal injection of 0.2 mL of 15 mg/mL D-luciferin under 1-2% inhaled isoflurane anesthesia. The bioluminescence signals were monitored using the IVIS system 2000 series (Xenogen Corp., Alameda, Calif., USA) consisting of a highly sensitive cooled CCD camera. Living Image software (Xenogen Corp.) was used to grid the imaging data and integrate the total bioluminescence signals in each boxed region. Data were analyzed using the total photon flux emission (photons/second) in the regions of interest (ROI). The results demonstrate that treatment with the h5D8 antibody promote tumor regression. Data are presented as mean±SEM.

### Example 11

### h5D8 Inhibits Tumor Growth in a Mouse Model of Glioblastoma Multiforme

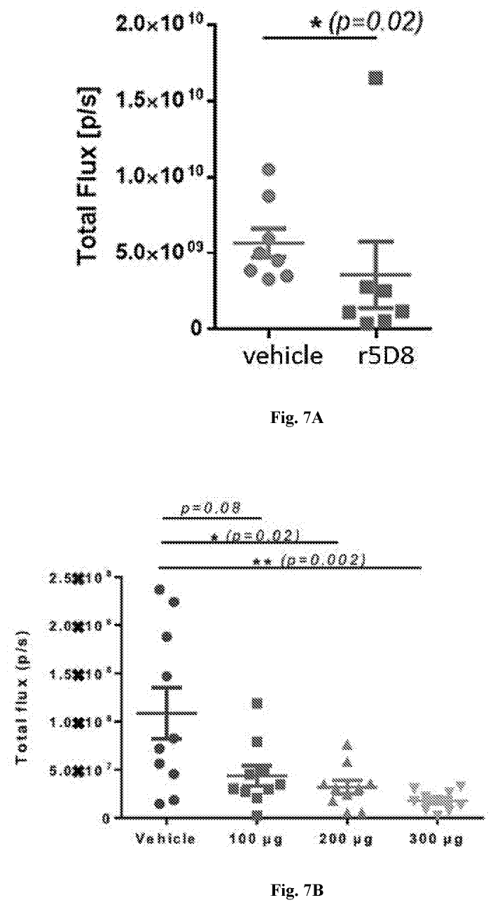

In an orthotopic GBM tumor model using a luciferase expressing human cell line U251, r5D8 significantly reduced tumor volumes in mice administered 300 μg r5D8 and h5D8 by intraperitoneal (IP) injection twice a week. Results of this study are shown in FIG. 7A (quantitation at day 26 post treatment). This experiment was also conducted using humanized h5D8 mice treated with 200 μg or 300 μg showed a statistically significant reduction in tumor after 7 days of treatment.

### Methods

U251 cells stably expressing luciferase were harvested, washed in PBS, centrifuged at 400 g for 5min, resuspended in PBS and counted with an automated cell counter (Countess, Invitrogen). Cells were kept on ice to maintain optimal viability. Mice were anaesthetized with intraperitoneal administration of Ketamine (Ketolar50®)/Xylacine (RompUng) (75 mg/kg and 10 mg/kg respectively). Each mouse was carefully placed in the stereotactic device and immobilized. Hair from the head was removed with depilatory cream, and the head skin was cut with a scalpel to expose the skull. A small incision was carefully made with a drill in the coordinates 1.8 mm lateral and 1mm anterior to the Lambda. 5μL of cells were inoculated using a Hamilton 30G syringe into the right corpus striatum, at 2.5 mm of depth. Head incision was closed with Hystoacryl tissue adhesive (Braun) and mice were injected with subcutaneous analgesic Meloxicam (Metacam®) (1 mg/kg). The final cell number implanted into each mouse was 3×10⁵.

Mice were treated twice a week with h5D8 administered intraperitoneally. Treatment was initiated on day 0, immediately after tumor cell inoculation. Mice received a total of 2 doses of h5D8 or vehicle control.

Body weight and tumor volume: Body weight was measured 2 times/week and tumor growth was quantified by bioluminescence on day 7 (Xenogen IVIS Spectrum). To quantify bioluminescence activity in vivo, mice were anaesthetized using isofluorane, and injected intraperitoneally with luciferin substrate (PerkinElmer) (167 μg/kg).

Tumor size as determined by bioluminescence (Xenogen IVIS Spectrum) was evaluated at day 7. The individual tumor measurements and mean±SEM for each treatment group were calculated. Statistical significance was determined by the unpaired non-parametric Mann-Whitney U-test.

### Example 12

### h5D8 Inhibits Tumor Growth in a Mouse Model of Ovarian Cancer

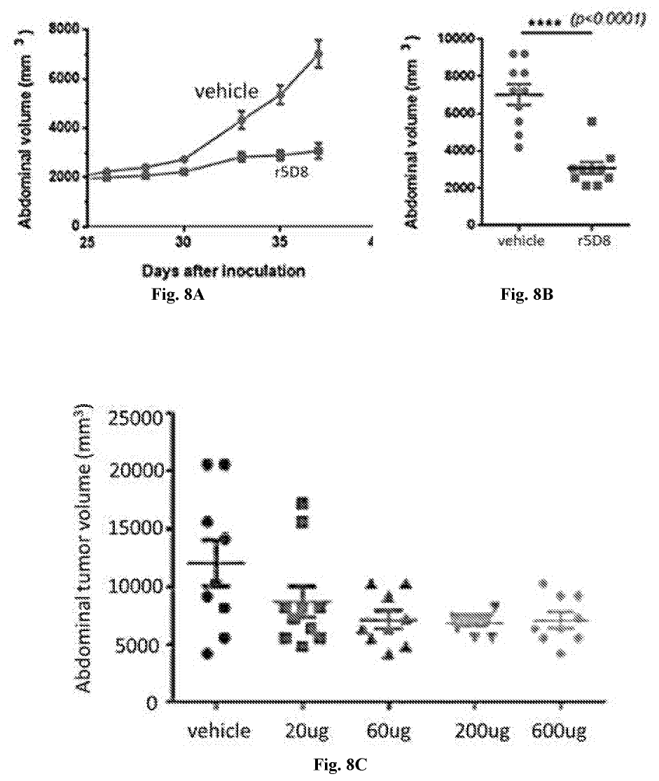

The efficacy of r5D8 was evaluated in two other syngeneic tumor models. In the ovarian orthotopic tumor model ID8, IP administration of 300 μg r5D8 twice weekly significantly inhibited tumor growth as measured by abdominal volume (FIG. 8A and 8B). Results in FIG. 8C show that h5D8 also reduced tumor volume at a dose of 200 μg and above.

### Methods

ID8 cells were cultured in Dulbecco's Modified Eagle Medium (DMEM) (Gibco, Invitrogen), supplemented with 10% Fetal Bovine Serum (FBS) (Gibco, Invitrogen), 40 U/mL Penicillin and 40 μg/mL Streptomycin (PenStrep) (Gibco, Invitrogen) and 0.25 μg/mL Plasmocin (Invivogen).

The ID8 cells were harvested, washed in PBS, centrifuged at 400 g for 5min and resuspended in PBS. Cells were kept on ice to maintain optimal viability and 200 μL of the cell suspension was injected intraperitoneally with a 27G needle. The final cell number implanted into mice was 5×10⁶.

Mice were treated twice weekly with h5D8 administered ip at different doses as indicated. Body weights were measured 2 times/week and tumor progression was monitored by measuring abdominal girth using a caliper (Fisher Scientific).

### Example 13

### r5D8 Inhibits Tumor Growth in a Mouse Model of Colorectal Cancer

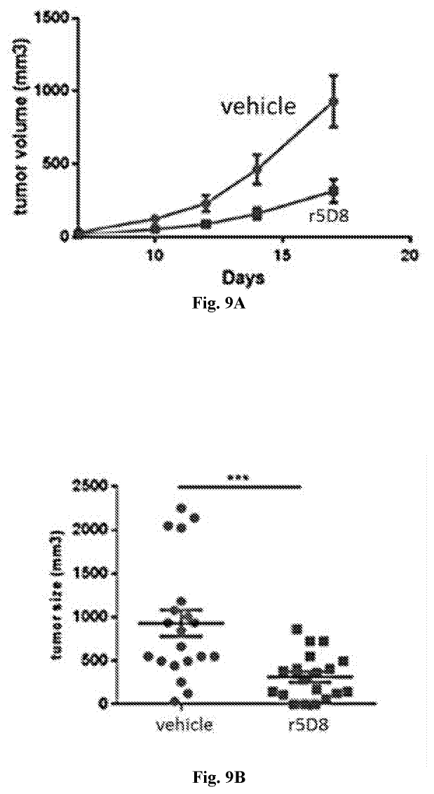

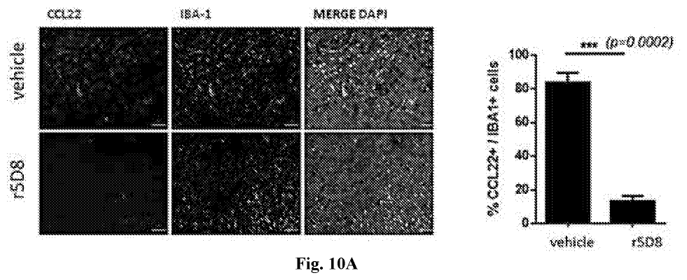

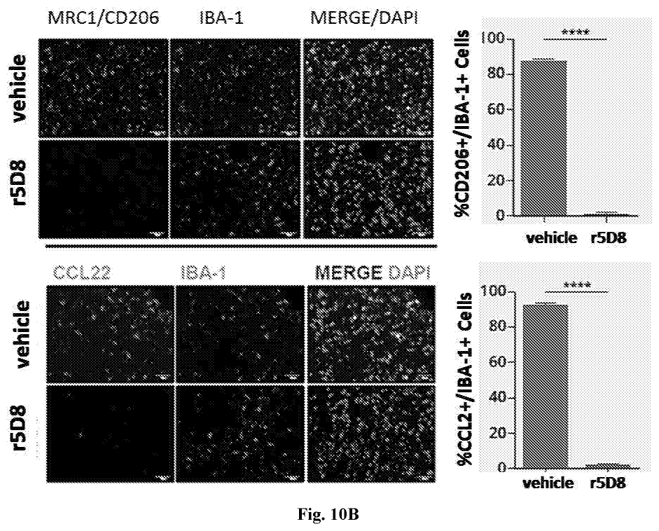

In mice with subcutaneous colon CT26 tumors, r5D8 (administered 300μg IP twice weekly) significantly inhibited tumor growth (FIG. 9A and 9B).

### Methods

CT26 cells were cultured in Roswell Park Memorial Institute medium (RPMI [Gibco, Invitrogen]), supplemented with 10% Fetal Bovine Serum (FBS), 40 U/mL penicillin and 40 1.1g/mL streptomycin (PenStrep) and 0.25 μg/mL Plasmocin.

CT26 cells (8×10⁵)were trypsinized, rinsed with PBS, centrifuged at 400 g for 5 minutes and resuspended in 100 !IL PBS. Cells were kept on ice to avoid cell death. The CT26 cells were administered to mice via subcutaneous injection using a 27G needle.

300μg r5D8, or vehicle control, was administered to the mice via intraperitoneal injection (IP) twice weekly from day 3 post CT26 cell implant.

Body weight and tumor volumes were measured three times per week. Tumor volume was measured using a caliper (Fisher Scientific).

### Example 14

### r5D8 Reduces Inflammatory Infiltration in Tumor Models

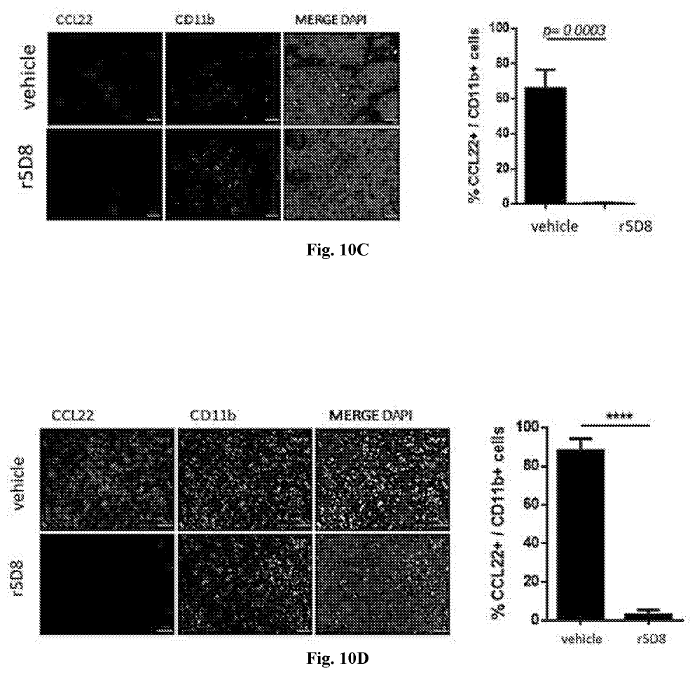

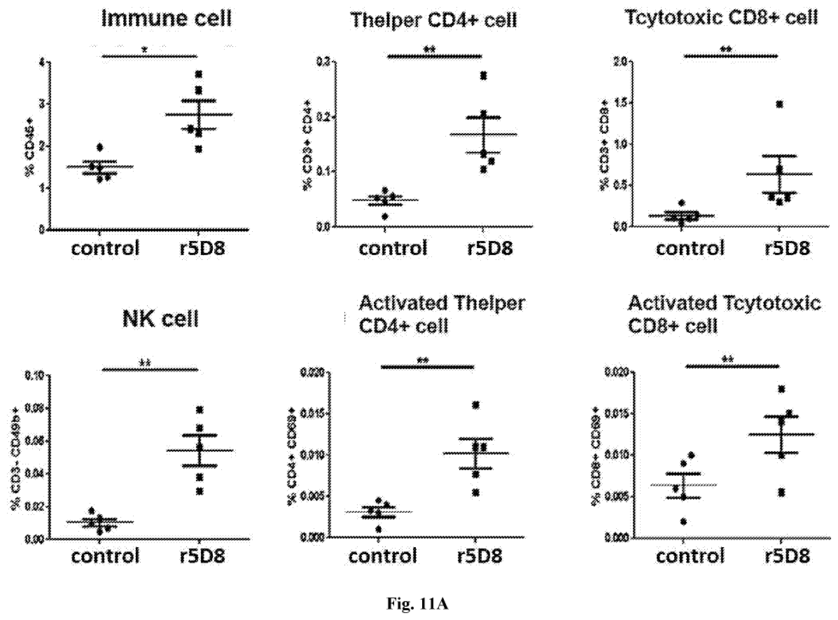

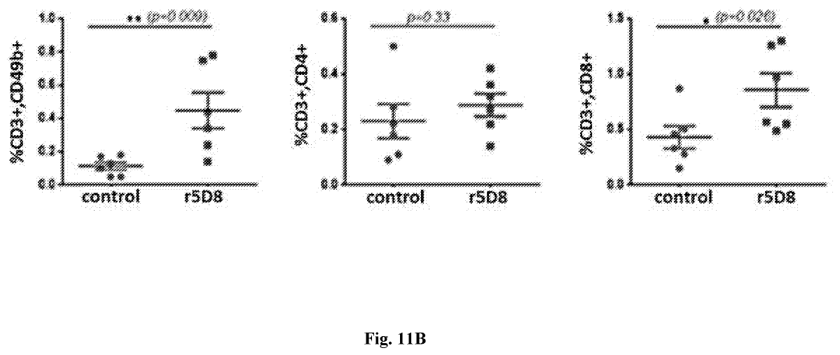

In the U251 GBM orthotopic model, expression of CCL22, a marker of M2 polarized macrophages, was significantly decreased in tumors treated with r5D8 as shown in FIG. 10A. This finding was also confirmed in a physiologically relevant organotypic tissue slice culture model using r5D8 in which three patient samples showed a significant decrease in CCL22 and CD206 (MRC1) expression (also a marker of M2 macrophages) after treatment, as shown in FIG. 10B (compare upper, control, to lower, treated, for both MRC1 and CCL22). Furthermore, r5D8 also decreased CCL22⁺M2 macrophages in syngeneic ID8 (FIG. 10C) and CT26 (FIG. 10D) tumors in immunocompetent mice.

### Example 15

### r5D8 Increases Non-Myeloid Effector Cells

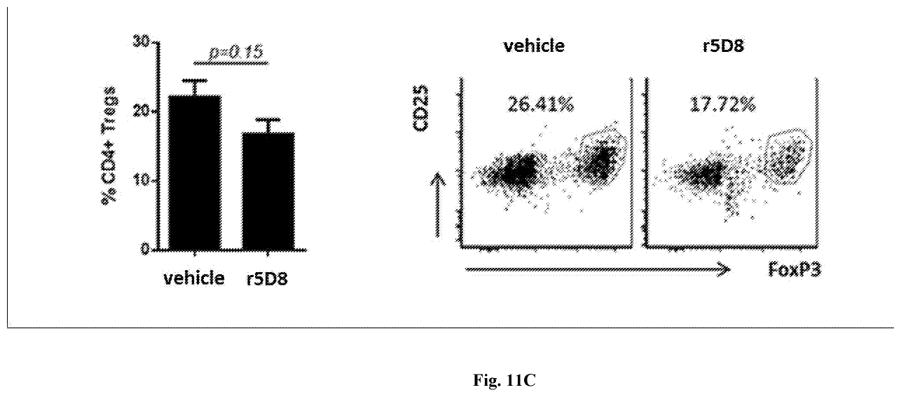

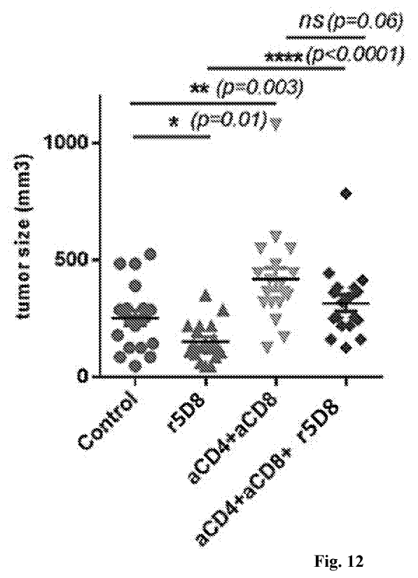

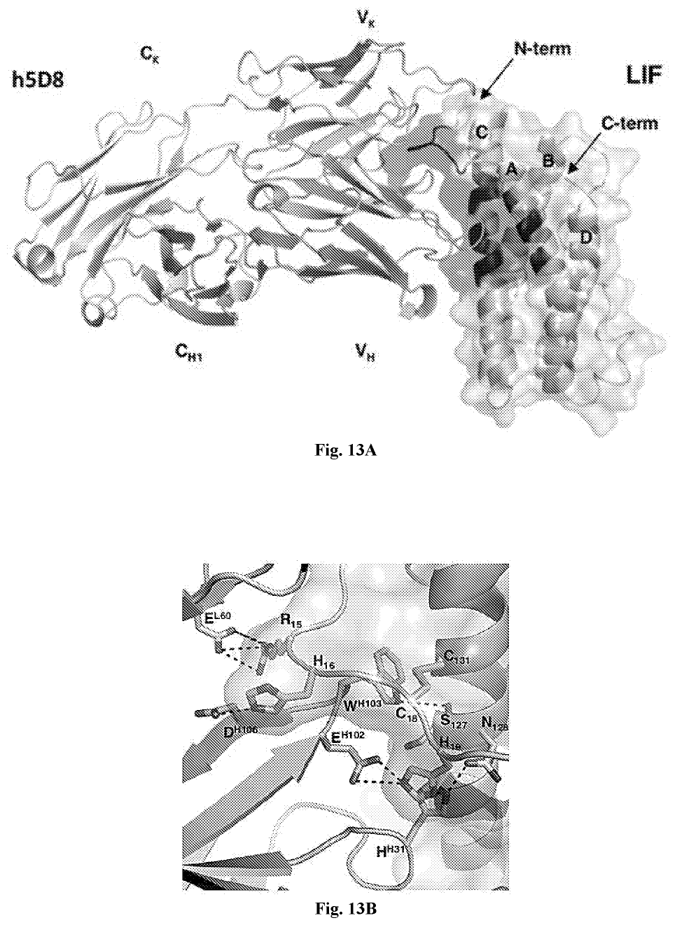

To investigate additional immune mechanisms, the effect of r5D8 on T cells and other non-myeloid immune effector cells within the tumor microenvironment were evaluated. In the ovarian orthotopic ID8 syngeneic model, r5D8 treatment resulted in an increase in intratumoral NK cells and an increase in total and activated CD4⁺ and CD8⁺ T cells as shown in FIG. 11A. Similarly, in the colon syngeneic CT26 tumor model, r5D8 increased intratumoral NK cells, increased CD4+ and CD8+ T cells and trended to decrease CD4⁺CD25⁺FoxP3⁺T-reg cells as shown in FIG. 11B. A trend for a decrease in CD4⁺CD25⁺FoxP3⁺T-reg cells was also observed in the syngeneic orthotopic KLN205 tumor model following r5D8treatment as shown in FIG. 11C. Consistent with a requirement for T cells to mediate efficacy, depletion of CD4⁺ and CD8⁺ T cells in the CT26 model inhibited the anti-tumor efficacy of r5D8 as shown in FIG. 12.

### Methods for T Cell Depletion

CT26 cells were cultured in RPMI culture medium (Gibco, Invitrogen), supplemented with 10% Fetal Bovine Serum (FBS [Gibco, Invitrogen]), 40 U/mL penicillin and 40 μg/mL streptomycin (PenStrep [Gibco, Invitrogen]) and 0.25 μg/mL Plasmocin (Invivogen). CT26 cells (5×10⁵) were collected, rinsed with PBS, centrifuged at 400 g for 5 minutes and resuspended in 100 μL PBS. Cells were kept on ice to avoid cell death. The CT26 cells were administered in both flanks to mice via subcutaneous injection using a 27G syringe. Mice were treated twice weekly with r5D8 administered intraperitoneally as indicated in the study design. Vehicle control (PBS), rat r5D8, and/or anti-CD4 and anti-CD8 was administered to the mice via intraperitoneal injection (IP) twice weekly as stated in the study design. All antibody treatments were administered concomitantly.

### Example 16

### Crystal Structure of h5D8 in Complex with Human LIF

The crystal structure of h5D8 was solved to a resolution of 3.1 angstroms in order to determine the epitope on LIF that h5D8 was bound to and to determine residues of h5D8 that participate in binding. The co-crystal structure revealed that the N-terminal loop of LIF is centrally positioned between the light and heavy chain variable regions of h5D8 (FIG. 13A). In addition, h5D8 interacts with residues on helix A and C of LIF, thereby forming a discontinuous and conformational epitope. Binding is driven by several salt-bridges, H-bonds and Van der Waals interactions (Table 7, FIG. 13B). The h5D8 epitope of LIF spans the region of interaction with gp130. See Boulanger, M. J., Bankovich, J., Kortemme, T., Baker, D. & Garcia, K. C. Convergent mechanisms for recognition of divergent cytokines by the shared signaling receptor gp130. Molecular cell 12, 577-589 (2003). The results are summarized below in Table 7 and depicted in FIG. 13.

TABLE 7
Summary of X-Ray crystal structure for h5D8 in complex with
human LIF

| LIF Residue | Interaction | h5D8 Residue |
|---|---|---|
| (epitope) | type | (paratope, Kabat numbering) |
| Ala13 | VDW | L-Tyr49, L-Asn53 |
| Ile14-O | HB | L-Ser50-OG |
| Ile | VDW | L-His30, L-Tyr32, L-Tyr49, L-Ser50 |
| | | H-Trp97 |
| Arg15-NE | SB | L-Glu55-OE1, L-Glu55-OE2 |
| Arg15-NH1 | SB | L-Glu55-OE1, L-Glu55-OE2 |
| Arg15-NH2 | SB | L-Glu55-OE1, L-Glu55-OE2 |
| Arg15-O | HB | L-Asn34-ND2 |
| Arg15 | VDW | L-Asn34, L-Leu46, L-Tyr49, L-Glu55, L- |
| | | Ser56 |
| | | H-Glu96, H-Trp97, H-Asp98, H-Leu99, |
| | | H-Asp101 |
| His16-NE2 | SB | H-Asp101-OD2 |
| His16 | VDW | L-Tyr32, L-Asn34, L-Met89 |
| | | H-Trp95, H-Glu96, H-Trp97, H-Asp101 |
| Pro17 | VDW | L-Tyr32, L-Ala91 |
| | | H-Trp97 |
| Cys18 | VDW | L-Tyr32 |
| | | H-Trp33, H-Trp97 |
| His19-NE2 | SB | H-Glu96-OE1, H-Glu96-OE2 |
| His19 | VDW | H-His31, H-Trp33, H-Glu96 |
| Asn20-OD1 | HB | H-Lys52-NZ |
| Asn20-ND2 | HB | H-Asp53-OD1 |
| Asn20 | VDW | H-Trp33, H-Lys52, H-Asp53 |
| Gln25-NE2 | HB | H-Asp53-OD2 |
| Gln25 | VDW | H-His31, H-Ser52C, H-Asp53 |
| Gln29 | VDW | H-His31 |
| Gln32 | VDW | H-Lys52B |
| Asp120-OD2 | HB | H-Ser30-OG |
| Asp120 | VDW | H-Thr28, H-Ser30 |
| Arg123-NE | HB | H-Thr28-OG |
| Arg123 | VDW | H-Thr28 |
| Gly124 | VDW | H-His31 |
| Leu125 | VDW | H-His31 |
| Ser127-OG | HB | H-Asp98-OD2 |
| Ser127-O | HB | H-Trp97-NE1 |
| Ser127 | VDW | H-His31, H-Trp97, H-Asp98 |
| Asn128-OD1 | HB | H-His31-NE2 |
| Asn128 | VDW | H-His31 |
| Leu130 | VDW | H-Trp97 |
| Cys131 | VDW | H-Trp97 |
| Cys134 | VDW | H-Trp97 |
| Ser135-O | HB | L-His30-NE2 |
| Ser135 | VDW | L-His30 |
| His138 | VDW | L-His30 |

VDW, Van der Waals low energy binding;
HB, hydrogen bond (medium (high energy binding) energy binding);
SB, salt bridge

### Methods

LIF was transiently expressed in HEK 293S (Gnt I^{-/-}) cells and purified using Ni-NTA affinity chromatography, followed by gel-filtration chromatography in 20 mM Tris pH 8.0 and 150 mM NaCl. The recombinant h5D8 Fab was transiently expressed in HEK 293F cells and purified using KappaSelect affinity chromatography, followed by cation exchange chromatography. Purified h5D8 Fab and LIF were mixed at a 1:2.5 molar ratio and incubated at room temperature for 30 min prior to deglycosylation using EndoH. Gel-filtration chromatography was subsequently used to purify the complex. The complex was concentrated to 20 mg/mL and set up for crystallization trials using sparse matrix screens. Crystals formed at 4° C. in a condition containing 19% (v/v) isopropanol, 19% (w/v) PEG 4000, 5% (v/v) glycerol, 0.095 M sodium citrate pH 5.6. The crystal diffracted to a resolution of 3.1 A at the 08ID-1 beamline at the Canadian Light Source (CLS). Data were collected, processed and scaled using XDS as per Kabsch et al. Xds. Acta crystallographica. Section D, Biological crystallography 66, 125-132 (2010). Structures were determined by molecular replacement using Phaser as per McCoy et al. Phaser crystallographic software. J Appl Crystallogr 40, 658-674 (2007). Several iterations of model building and refinement were performed using Coot and phenix.refine until the structures converged to an acceptable R_{work} and R_{free}. See Emsley et al. Features and development of Coot. Acta crystallographica. Section D, Biological crystallography 66, 486-501 (2010); and Adams, et al. PHENIX: a comprehensive Python-based system for macromolecular structure solution. Acta crystallographica. Section D, Biological crystallography 66, 213-221 (2010) respectively. The figures were generated in PyMOL (The PyMOL Molecular Graphics System, Version 2.0 Schrödinger, LLC).

### Example 17

### h5D8 has High Specificity for LIF

We sought to test binding of h5D8 to other LIF family members to determine the binding specificity. Using Octet96 analysis h5D8 binding to human LIF is approximately 100-fold greater than binding to LIFs highest homology IL-6 family member Oncostatin M (OSM) when both proteins are produced in E. coli. When both proteins are produced in a mammalian system h5D8 exhibits no binding to OSM. Data are summarized in Table 8.

TABLE 8
Summary of h5D8 Affinity Measurements for Cytokines as Measured by Octet

| | KD [M] | kon [1/Ms] | kdis [1/s] |
|---|---|---|---|
| h5D8 + huLIF | 4.3E−10 +/− 2.0E−11 | 3.1E+05 +/− 3.1E+03 | 1.3E−04 +/− 5.8E−06 |
| (E. coli) | | | |
| h5D8 + huLIF | 1.3E−09 +/− 7.2E−11 | 1.2E+05 +/− 1.3E+03 | 1.5E−04 +/− 8.5E−06 |
| (mammalian) | | | |
| h5D8 + huOSM | 3.6E−08 +/− 1.4E−09 | 8.5E+04 +/− 3.1E+03 | 3.1E−03 +/− 4.1E−05 |
| (E. coli) | | | |
| h5D8 + huOSM | ND | ND | ND |
| (mammalian) | | | |
| h5D8 +huIL-6 | ND | ND | ND |
| (E. coli) | | | |

ND = no binding

### Methods

Octet Binding Experiments: Reagents were used and prepared as per manufacturer's provided manual. A Basic Kinetics Experiment was performed using Octet Data Acquisition software ver. 9.0.0.26 as follows: Setup of sensors/program: i) Equilibration (60 seconds); ii) Loading (15 seconds); iii) Baseline (60 seconds); iv) Association (180 seconds); and v) Dissociation (600 seconds)

Octet Affinity of h5D8 for cytokines: A Basic Kinetics Experiment was performed using Octet Data Acquisition software ver. 9.0.0.26 as follows: Amine Reactive 2ndGeneration Biosensors (AR2G) were hydrated for a minimum of 15 minutes in water. Amine conjugation of h5D8 to the biosensors was performed according to ForteBio Technical Note 26 (please see References) using the Amine Coupling Second Generation Kit. Dip steps were as performed at 30° C., 1000 rpm as follows: i) 60 seconds Equilibration in water; ii) 300 seconds Activation in 20 mM ECD, 10mM sulfo-NHS in water; iii) 600 second Immobilization of 10 μg/ml h5D8 in 10 mM Sodium Acetate, pH 6.0; iv) 300 seconds Quench in 1M Ethanolamine, pH 8.5; v) 120 seconds Baseline in water. Kinetics experiments were then performed with the following Dip and Read steps at 30° C., 1000rpm: vi) 60 seconds Baseline in lx kinetics buffer; vii) 180 seconds Association of appropriate serial dilutions of a cytokine in lx kinetics buffer; viii) 300 seconds Dissociation in 1X kinetics buffer; ix) Three Regeneration/Neutralization cycles alternating between 10mM glycine pH 2.0 and lx kinetics buffer respectively (5 seconds in each for 3 cycles). Following regeneration, the biosensors were reused for subsequent binding analyses.

Human recombinant LIF produced from mammalian cells was from ACROBiosystems (LIF-H521b); human recombinant OSM produced in mammalian cells was from R & D (8475-OM/CF); and human recombinant OSM produced in E. coli cells was from R & D (295-OM-050/CF).

### Example 18

### Crystal Structure of h5D8 Fab

Five crystal structures of the h5D8 Fab under a wide spectrum of chemical conditions were determined. The high resolutions of these structures indicate that the conformations of CDR residues are associated with minor flexibility, and are highly similar in different chemical environments. A unique feature of this antibody is the presence of a non-canonical cysteine in position 100 of the variable heavy region. Structure analysis shows that the cysteine is unpaired and largely inaccessible to the solvent.

H5D8 Fab was obtained by papain digestion of its IgG, followed by purification using standard affinity, ion exchange and size chromatography techniques. Crystals were obtained using vapor diffusion methods and allowed to determine five crystal structures ranging between 1.65 A to 2.0 A in resolution. All structures were solved in the same crystallographic space group and with similar unit cell dimensions (P212121, a˜53.8 Å, b˜66.5 Å, c˜143.3 Å), despite crystallization conditions ranging across five different pH levels: 5.6, 6.0, 6.5, 7.5 and 8.5. As such, these crystal structures allow for comparison of the three-dimensional disposition of h5D8 Fab unimpeded by crystal packing artefacts and across a wide spectrum of chemical conditions.

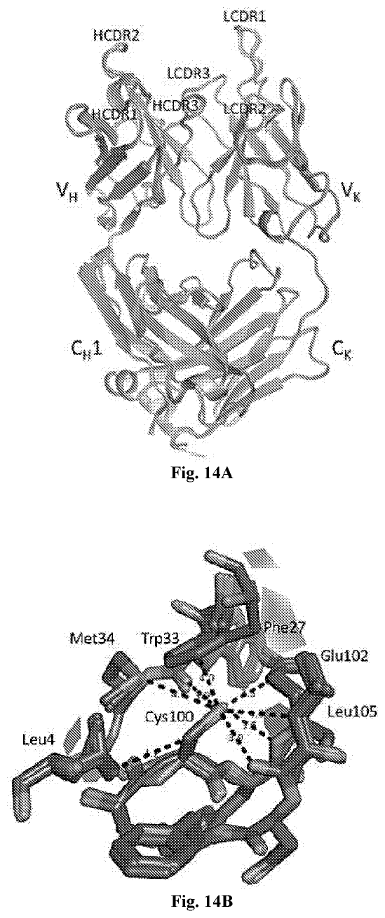

Electron density was observed for all complementarity determining region (CDR) residues, which were subsequently modeled. Noticeably, LCDR1 and HCDR2 adopted elongated conformations that together with shallow LCDR3 and HCDR3 regions formed a binding groove at the center of the paratope (FIG. 14A). The five structures were highly similar across all residues, with all-atoms root mean square deviations ranging between 0.197 Å and 0.327 Å (FIG. 14A). These results indicated that the conformations of CDR residues were maintained in various chemical environments, including pH levels ranging between 5.6 and 8.5 and ionic strengths ranging between 150 mM and 1 M. Analysis of the electrostatic surface of the h5D8 paratope revealed that positively and negatively charged regions equally contributed to hydrophilic properties, with no prevalent hydrophobic patches. h5D8 has the uncommon feature of a non-canonical cysteine at the base of HCDR3 (Cys100). In all five structures, this free cysteine is ordered and does not form any disulfide scrambles. Additionally, it is not modified by the addition of Cys (cysteinylation) or glutathione (glutathiolation) and makes Van der Waals interactions (3.5-4.3 Å distances) with main chain and side chain atoms of Leu4, Phe27, Trp33, Met34, Glu102 and Leu105 of the heavy chain (FIG. 14B). Finally, Cys100 is a predominantly buried structural residue that appears to be involved in mediating the conformations of CDR1 and HCDR3. It is thus unlikely to have reactivity with other cysteines, as observed by a homogeneous disposition of this region in our five crystal structures.

### Methods

H5D8-1 IgG was obtained from Catalent Biologics and was formulated in 25 mM histidine, 6% sucrose, 0.01% polysorbate 80, at pH 6.0. The formulated IgG was extensively buffer-exchanged into PBS using a 10K MWCO concentrator (Millipore) prior to digestion with 1:100 microgram papain (Sigma) for 1 hour at 37° C. in PBS, 1.25 mM EDTA, 10 mM cysteine. The papain-digested IgG was flown through a Protein A column (GE Healthcare) using an AKTA Start chromatography system (GE Healthcare). The Protein A flow-through, which contained the h5D8 Fab was recovered and buffer-exchanged into 20 mM sodium acetate, pH 5.6 using a 10K MWCO concentrator (Millipore). The resulting sample was loaded onto a Mono S cation exchange column (GE Healthcare) using an AKTA Pure chromatography system (GE Healthcare). Elution with a gradient of 1 M potassium chloride resulted in a predominant h5D8 Fab peak that was recovered, concentrated and purified to size homogeneity using a Superdex 200 Increase gel filtration column (GE Healthcare) in 20 mM Tris-HC1, 150 mM sodium chloride, at pH 8.0. The high purity of the h5D8 Fab was confirmed by SDS-PAGE under reducing and non-reducing conditions.

Purified h5D8 Fab was concentrated to 25 mg/mL using a 10K MWCO concentrator (Millipore). An Oryx 4 dispenser (Douglas Instruments) was used to set up vapor diffusion crystallization experiments with sparse matrix 96-conditions commercial screens JCSG TOP96 (Rigaku Reagents) and MCSG-1 (Anatrace) at 20° C. Crystals were obtained and harvested after four days in the following five crystallization conditions: 1) 0.085 M sodium citrate, 25.5% (w/v) PEG 4000, 0.17 M ammonium acetate, 15% (v/v) glycerol, pH 5.6; 2) 0.1 M MES, 20% (w/v) PEG 6000, 1 M lithium chloride, pH 6.0; 3) 0.1 M IVIES, 20% (w/v) PEG 4000, 0.6 M sodium chloride, pH 6.5; 4) 0.085 M sodium HEPES, 17% (w/v) PEG 4000, 8.5% (v/v) 2-propanol, 15% (v/v) glycerol, pH 7.5; and 5) 0.08 M Tris, 24% (w/v) PEG 4000, 0.16 M magnesium chloride, 20% (v/v) glycerol, pH 8.5. Prior to flash-freezing in liquid nitrogen, mother liquors containing the crystals were supplemented with 5-15% (v/v) glycerol or 10% (v/v) ethylene glycol, as required. Crystals were subjected to X-ray synchrotron radiation at the Advanced Photon Source, beamline 23-ID-D (Chicago, IL) and diffraction patterns were recorded on a Pilatus3 6M detector. Data were processed using XDS and structures were determined by molecular replacement using Phaser. Refinement was carried out in PHENIX with iterative model building in Coot. Figures were generated in PyMOL. All software were accessed through SBGrid.

### Example 19

### Mutations at Cysteine 100 of h5D8 Preserve Binding

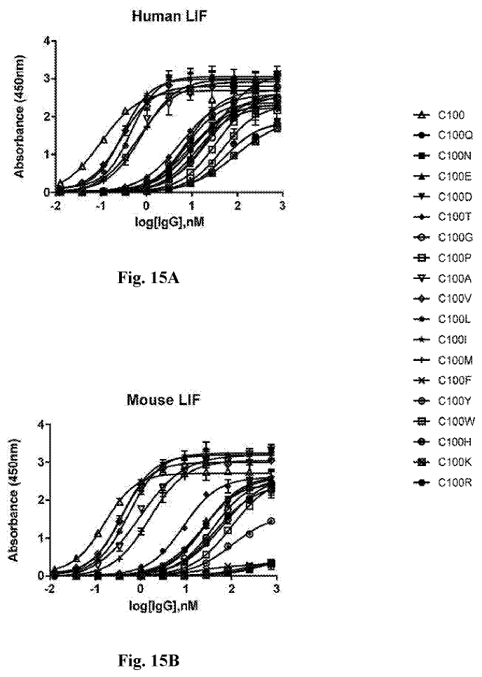

Analysis of h5D8 revealed a free cysteine residue at position 100 (C100) in the variable region of the heavy chain. H5D8 variants were generated by substituting C100 with each naturally occurring amino acid in order to characterize binding to and affinity for human and mouse LIF. Binding was characterized using ELISA and Octet assay. Results are summarized in Table 9. ELISA EC50 curves are shown in FIG. 15 (FIG. 15A human LIF and FIG. 15B Mouse LIF).

TABLE 9
Summary of affinities determined by Octet assay and EC50 determined by ELISA

| | Affinity/k_{D} (M) | | Binding EC50 (nM) | |
|---|---|---|---|---|
| Mutation | human LIF | mouse LIF | human LIF | mouse LIF |
| C100 | <1.0E−12 ± 2.252E−11 | 9.946E−11 ± 8.272E−12 | 0.09878 | 0.1605 |
| C100S | 8.311E−10 ± 5.886E−11 | 2.793E−09 ± 5.925E−11 | n.d. | n.d. |
| C100Q | 3.87E−09 ± 1.55E−10 | 2.84E−09 ± 4.85E−11 | 10.18 | 26.33 |
| C100N | 5.59E−09 ± 1.01E−10 | 6.68E−09 ± 9.8E−11 | 13.18 | 45.87 |
| C100E | 2.67E−09 ± 4.64E−11 | 4.1E−09 ± 7.56E−11 | 7.179 | 25.3 |
| C100D | 2.02E−09 ± 8.08E−11 | 6.49E−09 ± 7.16E−11 | 11.89 | 22.88 |
| C100T | 4.36E−10 ± 2.1E−11 | 1.02E−09 ± 1.77E−11 | 5.575 | 8.753 |
| C100G | 2.49E−09 ± 4.2E−11 | 3.33E−09 ± 5.42E−11 | 21.94 | 40.17 |
| C100P | 2.74E−10 ± 2.97E−10 | <1.0E−12 ± 7.64E−10 | 34.44 | 101.9 |
| C100A | <1.0E−12 ± 2.713E−11 | <1.0E−12 ± 1.512E−11 | 0.6705 | 0.9532 |
| C100V | <1.0E−12 ± 1.805E−11 | <1.0E−12 ± 8.086E−11 | 0.2785 | 0.3647 |
| C100L | <1.0E−12 ± 1.963E−11 | 1.998E−10 ± 1.055E−11 | 0.454 | 0.547 |
| C100I | <1.0E−12 ± 1.424E−11 | 3.361E−11 ± 7.545E−12 | 0.299 | 0.3916 |
| C100M | 1.155E−09 ± 3.400E−11 | 2.676E−09 ± 2.449E−11 | 0.7852 | 1.563 |
| C100F | 4.376E−09 ± 1.127E−10 | 1.147E−08 ± 9.099E−11 | 8.932 | 21.53 |
| C100Y | 1.444E−08 ± 1.159E−09 | 2.514E−08 ± 2.047E−09 | n.d. | n.d. |
| C100W | 2.508E−08 ± 7.036E−09 | 4.819E−08 ± 4.388E−09 | n.d. | n.d. |
| C100H | 1.304E−10 ± 1.416E−10 | 4.284E−09 ± 1.231E−10 | 8.254 | n.d. |
| C100K | 7.477E−08 ± 1.581E−09 | 6.053E−08 ± 2.589E−09 | n.d. | n.d. |
| C100R | 1.455E−07 ± 6.964E−09 | 5.142E−08 ± 3.247E−09 | n.d. | n.d. |

### Methods

ELISA: Binding of h5D8 C100 variants to human and mouse LIF was determined by ELISA. Recombinant human or mouse LIF protein was coated on Maxisorp 384-well plates at 1 ug/mL overnight at 4° C. Plates were blocked with lx blocking buffer for 2 hours at room temperature. Titrations of each h5D8 C100 variants were added and allowed to bind for 1 hour at room temperature. Plates were washed three times with PBS+0.05% Tween-20. HRP-conjugated anti-human IgG was added and allowed to bind for 30 min at room temperature. Plates were washed three times with PBS+0.05% Tween-20 and developed using lx TMB substrate. The reaction was stopped with 1M HC1 and absorbance at 450 nm was measured. Generation of figures and non-linear regression analysis was performed using Graphpad Prism.

Octet RED96: The affinity of h5D8 C100 variants to human and mouse LIF was determined by BLI using the Octet RED96 system. h5D8 C100 variants were loaded onto Anti-Human Fc biosensors at 7.5 ug/mL following a 30 second baseline in lx kinetics buffer. Titrations of human or mouse LIF protein were associated to the loaded biosensors for 90 seconds and allowed to dissociate in lx kinetics buffer for 300 seconds. KDs were calculated by the data analysis software using a 1:1 global fit model.

### Example 20

### h5D8 Blocks Binding of LIF to gp130 In Vitro

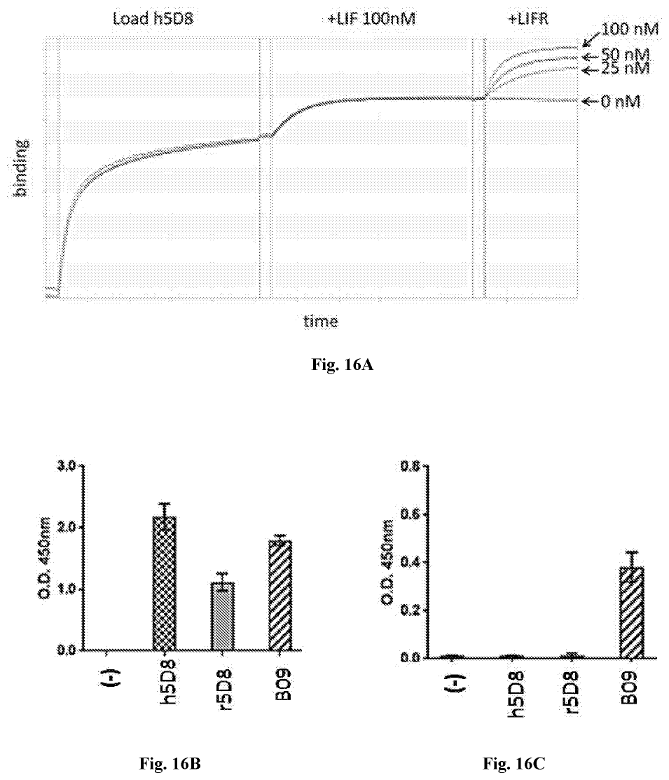

To determine whether h5D8 prevented LIF from binding to LIFR, a molecular binding assay using the Octet RED 96 platform was performed. H5D8 was loaded onto AHC biosensors by anti-human Fc capture. Then, the biosensors were dipped in LIF and, as expected, association was observed (FIG. 16A, middle third). Subsequently, the biosensors were dipped in different concentrations of LIFR. A dose-dependent association was observed (FIG. 16A, right third). The control experiment demonstrated that this association was LIF-specific (not shown), and not due to a non-specific interaction of LIFR with h5D8 or with the biosensors.

To further characterize the binding of h5D8 and LIF, a series of ELISA binding experiments was conducted. H5D8 and LIF were pre-incubated and were then introduced to plates coated with either recombinant human LIFR (hLIFR) or gp130. The lack of binding between the h5D8/LIF complex and the coated substrate would indicate that h5D8 in some way disrupted the binding of LIF to the receptor. Additionally, control antibodies that either did not bind LIF (isotype control, indicated by (-)) or that bind LIF at known binding sites (B09 does not compete with either gp130 or LIFR for LIF binding; r5D8 is the rat parental version of h5D8) were also used. The ELISA results demonstrated that the h5D8/LIF complex was able to bind hLIFR (as was r5D8/LIF complex), indicating that these antibodies did not prevent the LIF/LIFR association (FIG. 16A). In contrast, the h5D8/LIF complex (and a r5D8/LIF complex) was not able to bind recombinant human gp130 (FIG. 16B). This indicates that the gp130 binding site of LIF was affected when LIF was bound to h5D8.

### Example 21

### LIF and LIFR Expression in Human Tissues

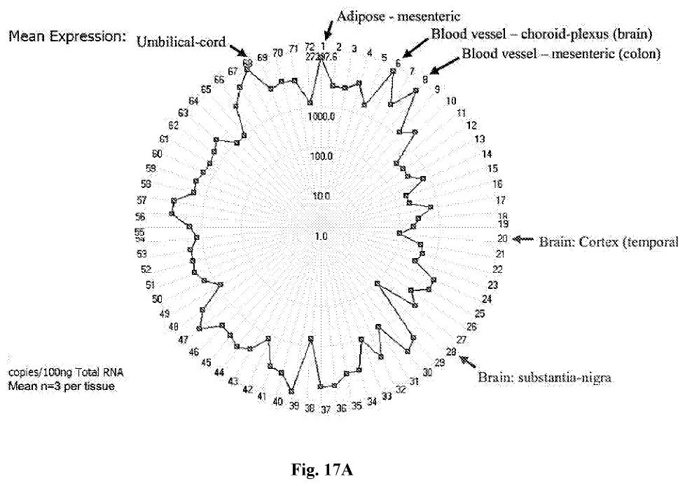

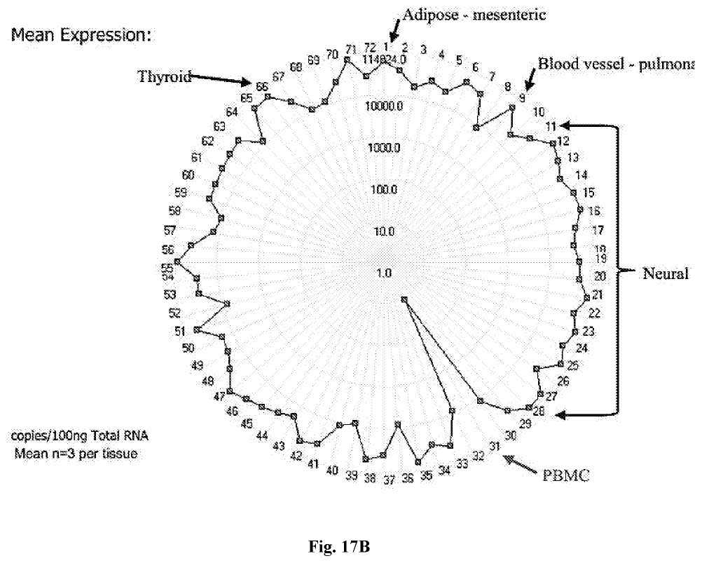

Quantitative real-time PCR was performed on many different types of human tissue in order to determine expression levels of LIF and LIFR. The mean expression levels shown in FIG. 17A and 17B are given as copies per 10Ong of total RNA. Most tissues expressed at least 100 copies per 10Ong of total RNA. LIF mRNA expression was highest in human adipose tissue (mesenteric-ileum [1]), blood-vessel tissue (choroid-plexus [6] and mesenteric [8]) and umbilical cord [68] tissue and lowest in brain tissue (cortex [20] and substantia-nigra [28]). LIFR mRNA expression was highest in human adipose tissue (mesenteric-ileum [1]), blood vessel tissue (pulmonary [9]), brain tissue [11-28] and thyroid [66] tissue and was lowest in PBMCs [31]. LIF and LIFR mRNA expression levels in cynomolgus tissues were similar to those observed in human tissues, wherein LIF expression was high in adipose tissue and LIFR expression was high in adipose tissue and low in PBMCs (data not shown).

The tissue numbering for FIG. 17A and FIG. 17B is: 1—adipose (mesenteric-ileum); 2—adrenal gland; 3—bladder; 4—bladder (trigone); 5—blood-vessel (cerebral: middle-cerebral-artery); 6—blood vessel (choroid-plexus); 7—blood vessel (coronary artery); 8—blood vessel (mesenteric (colon)); 9—blood vessel (pulmonary); 10—blood vessel (renal); 11—brain (amygdala); 12—brain (caudate); 13—brain (cerebellum); 14 brain—(cortex: cingulate-anterior); 15—brain (cortex: cingulate-posterior); 16—brain (cortex: frontal-lateral); 17—brain (cortex: frontal-medial); 18—brain (cortex: occipital); 19—brain (cortex: parietal); 20—brain (cortex: temporal); 21—brain (dorsal-raphe-nucleus); 22—brain (hippocampus); 23—brain (hypothalamus: anterior); 24—brain (hypothalamus: posterior); 25—brain (locus coeruleus); 26—brain (medulla oblongata); 27—brain (nucleus accumbens); 28—brain (substantia nigra); 29—breast; 30—caecum; 31—peripheral blood mononuclear cell (PBMCs); 32—colon; 33—dorsal root ganlia (DRG); 34—duodenum; 35—fallopian tube; 36—gallbladder; 37—heart (left atrium); 38—heart (left ventricle); 39—ileum;40—jejunum;41—kidney (cortex); 42—kidney (medulla);43—kidney (pelvis); 44—liver (parenchyma); 45—liver (bronchus: primary); 46—liver (bronchus: tertiary); 47—lung (parenchyma); 48—lymph gland (tonsil); 49—muscle (skeletal); 50—esophagus; 51—ovary; 52—pancreas; 53—pineal gland; 54—pituitary gland; 55—placenta; 56—prostate; 57—rectum; 58—skin (foreskin); 69—spinal cord; 60—spleen (parenchyma); 61—stomach (antrum); 62—stomach (body); 63—stomach (fundus); 64—stomach (pyloric canal); 65—testis; 66—thyroid gland; 67—trachea; 68—umbilical cord; 69—ureter; 70—uterus (cervix); 71—uterus (myometrium); and 72—vas deferens.

### Example 22

### Dose Selection, Dose Increments and Flat Dosing

Anti-LIF antibody dose selection, dose increments and flat dosing are described below. Mice and cynomolgus monkeys were used for the safety evaluation of h5D8.

No treatment-related adverse effects were observed in 4-week GLP toxicity studies in mice and monkeys which received weekly IV dosing up to 100 mg/kg. Thus, the highest non-severely toxic dose (HNSTD) is >100 mg/kg and the no-observed-adverse-effect-level (NOAEL) was established as 100 mg/kg IV in both species under the conditions of the studies. The dosage was scaled to establish a human equivalent dose (HED) A body surface area (BSA)-based scaling approach was adopted for the estimation of the HED Based on these GLP toxicology studies a maximum recommended starting dose (MRSD) was estimated as shown below:

0.81 mg/kg IV HED from mouse NOAEL with 10-fold safety factor

>10 mg/kg IV based on 1/10 the severely toxic dose in mice

3.2 mg/kg IV HED from cynomolgus monkey NOAEL with 10-fold safety factor

>16.7 mg/kg IV based on 1/6 the HNSTD

Based on the toxicology studies, and taking a conservative approach for an advanced cancer patient population in the Phase 1 study, a MRSD of 1 mg/kg (or 75 mg flat dose) IV was supported by the data.

The pharmacologically active dose (PAD) has also been considered in setting the MRSD. Based on pharmacology, PK and LIF level data in mouse pharmacology models available to date, the following approach was used to estimate the PAD. Based on the dose-response in the U251 mouse xenograft model, the optimal efficacious dose was considered to be about 300 μg IP twice weekly; this dose level was associated with a trough serum level before the last dose of about 230 μg/mL. There was evidence that maximal levels of serum LIF levels had been achieved at this 300 μg dose in this model, which was also supported by serum LIF level data in the mouse GLP toxicity study at doses of 10, 30 and 100 mg/kg. Using a PK model based on a 2-compartmental model fitted to the monkey PK data and scaled for humans, a clinical dose of 1500 mg every 3 weeks would provide a C _{trough} of about 500 μg/mL. Similarly, the minimally effective dose of 20 μg twice weekly in this U251 mouse xenograft model was associated with a trough serum level before the last dose of about 20 μg/mL; there was evidence that only about 50% of maximal serum LIF level was achieved at this 20-μg dose, supported by evidence of minimal LIF levels at a dose of 0.5 mg/kg IV in the mouse PK-tolerability study. A clinical dose of 75 mg every 3 weeks would provide a C _{trough} of about 25 μg/mL. Additional PK-PD (LIF levels) data available from mouse syngeneic models supported the PAD derived from the U251 mouse xenograft model.

Thus, a starting dose of 75 mg i.v. was considered appropriate based on both the toxicology data in mice and monkeys and the minimal effective dose in a mouse xenograft model. A maximum clinical dose of 1500 to 2000 mg was supported by the toxicology data. A flat-dosing approach was appropriate based on the observation of a linear PK in animal models, in conjunction with the absence of test-article related adverse findings.

### Example 23

### Assay to Determine Levels of Leukemia Inhibitory Factor

### Preparation of MSD Plates

MSD plates (Meso Scale Diagnostics; Cat. No. R93BA-1) are spot-coated with freshly diluted rabbit A4 monoclonal capture antibody (5μ.1 of 100μg/ml in PBS+0.03% Triton X-100 per well of a 96-well plate) and incubated overnight at room temperature. The next day the plate is blocked with a blocking solution and the sample preparation is performed. Plates may be used immediately, or dried and refrigerated for future use.

### Sample Preparation and Standards

Quality controls are prepared in bulk in 100% Normal human serum or plasma (NHS or NHP) pool (matrix) containing a saturating amount of h5D8 therapeutic antibody (20ug/m1) and recombinant human LIF (rhLIF; ACRO Biosystems; Cat. No. LIF-H521b) and may be stored at −80 ° C. and thawed prior to use. The standards are prepared (double concentrated) in normal human serum or plasma containing saturating amounts of therapeutic antibody with rhLIF in a geometric dilution series. Standards may be stored at −80 ° C. and thawed prior to use. Thawed quality controls and standard series are pipetted into the wells of the deep-well plate. If previously frozen, subject samples of plasma or serum are thawed. Standards, quality controls and samples are diluted 1: 2 with Diluent 2 (Meso Scale Diagnostics; Cat. No. R51BB-4) at a final concentration 50% matrix and containing l0ug/ml therapeutic antibody, and incubated at room temperature for 30 minutes.

### Assay

Quality controls and samples are transferred in triplicate to the coated and blocked MSD plate. After incubation at room-temperature for 1 hour, MSC-1/LIF complexes are removed by a wash step. Sulfo-Tag™ mouse monoclonal detection antibody (Biolegend; clone M017C3; Cat. No. 530301) is added and after incubation unbound material is removed by another washing step. The bound MSC-1/LIF complex is detected by the Sulfo-Tag™ by an MSD 5600 instrument. The intensity quantified by the system is proportional to the amount of bound antibody complexes and is expressed as RLU (Relative Light Units). Total LIF concentrations are calculated in pg/ml by interpolation against the standard curve, and are multiplied by the dilution factor 2 to arrive at the total LIF concentration in the plasma or serum sample.

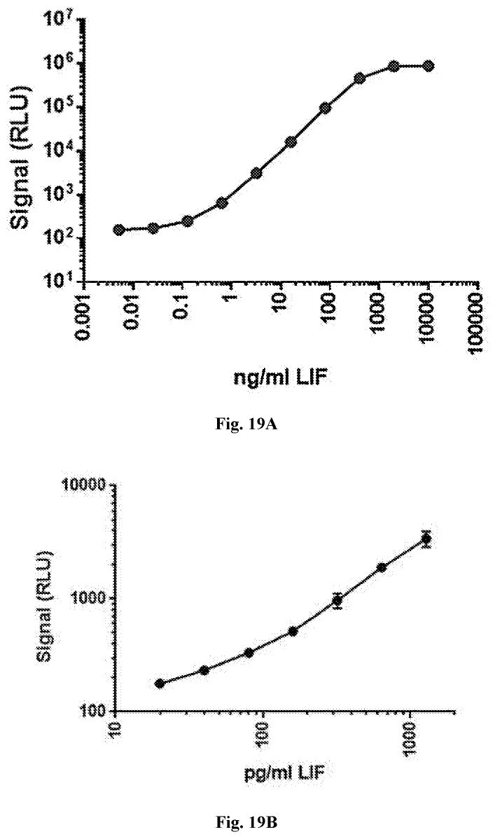

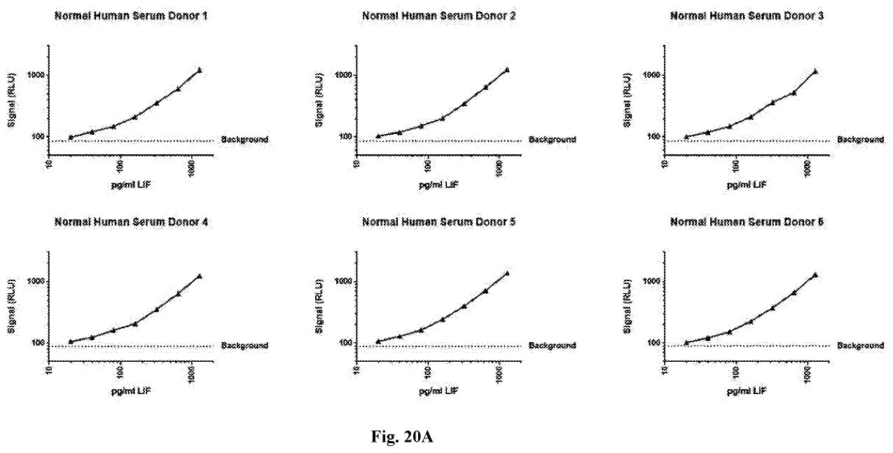

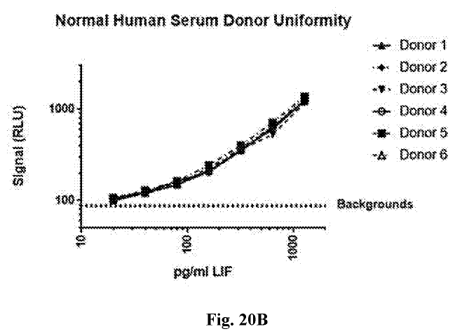

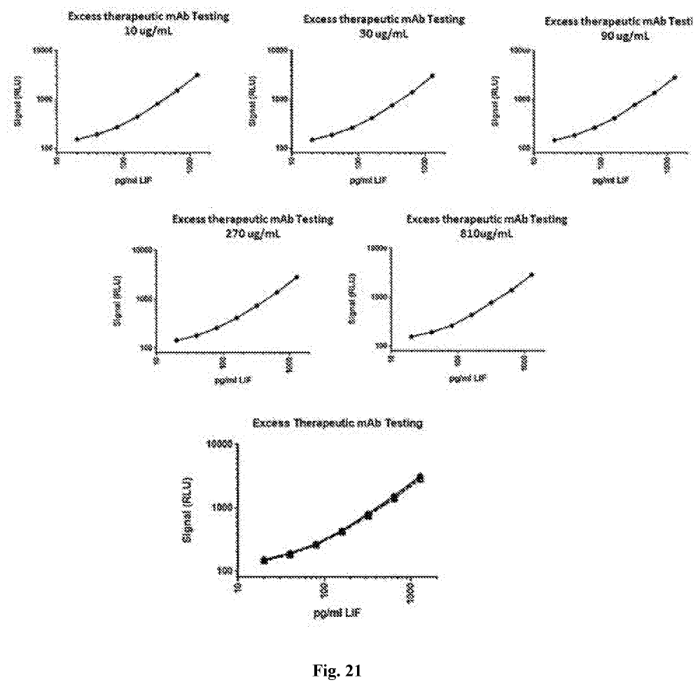

This assay has a wide dynamic range as shown in FIGS. 19A and 19B; high uniformity as shown in FIGS. 20 A and B; and is stable over a high concentration of therapeutic antibody concentrations as shown in FIG. 21.

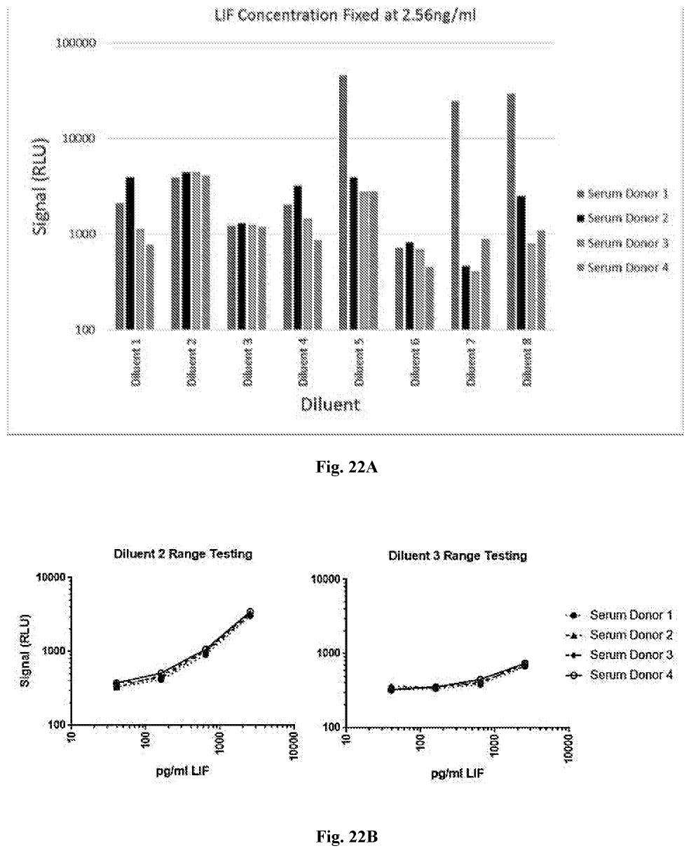

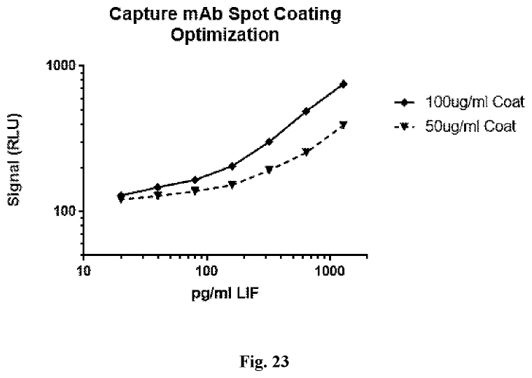

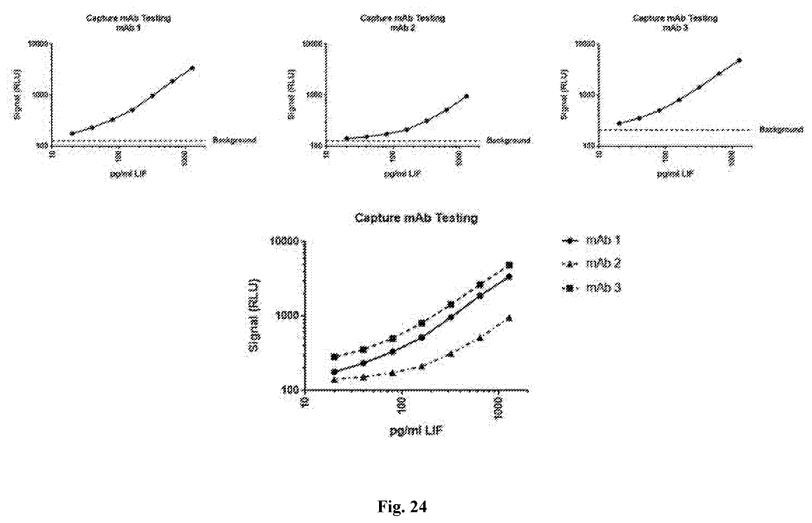

This assay was subjected to much optimization including the diluent used as shown in FIG. 22A and 22B; spot coating concentration of the capture antibody as shown in FIG. 23; and detection antibody used as shown in FIG. 24.

### Example 24

### Epitopes Bound by Therapeutic and Detection Antibodies

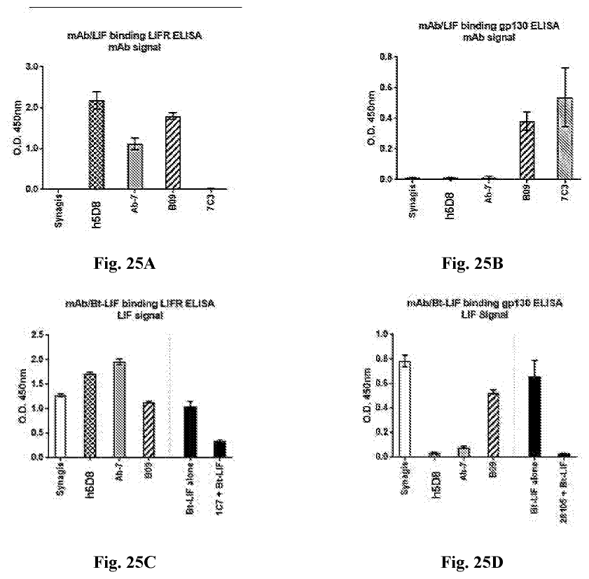

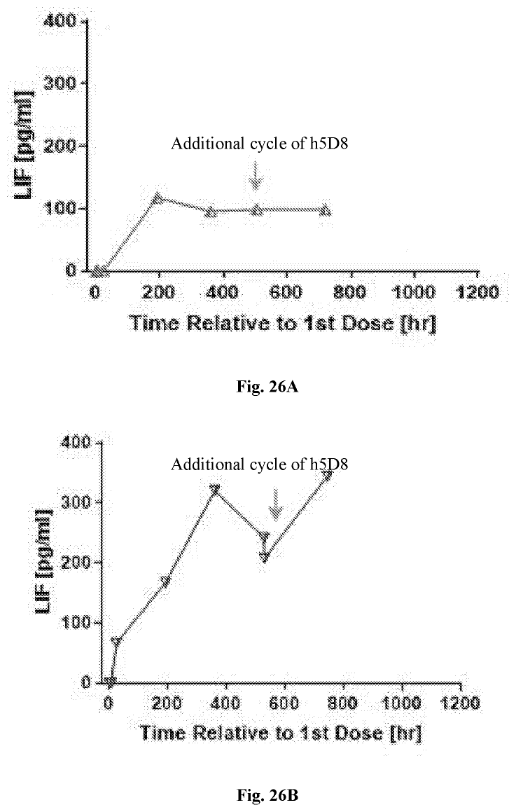

In order to utilize a capture ELISA to measure engagement of target by a therapeutic antibody, the capture and detection antibodies must bind to distinct regions of the target. Additionally, if total target (both bound and unbound by therapeutic antibody) is to be measured all three antibodies involved (therapeutic, detection and capture) must bind the target and not interfere with binding of the other two antibodies. To this end we sought to determine binding in ELISA assays of preformed LIF/antibody complexes. As shown in FIGS. 25A and 25B detecting antibody M017C3 likely binds to the a portion of LIF that interacts with the LIF receptor since LIF complexed with 7C3 binds to gp130 coated plates and not LIF receptor coated plates. Therapeutic antibody h5D8 likely binds to a portion of LIF that interacts with gp130 since LIF complexed with h5D8 binds to LIF receptor coated plates and not gp130 coated plates. Similar results are seen when the LIF is biotinylated and detected using avidin labeled HRP as shown in FIGS. 25C and 25D.

The capture antibody A4 (set forth in SEQ ID NOs: 41 and 42) was determined by screening potential antibodies using competitive binding assays against LIF. These experiments reveled antibody A4 as exhibited high binding to LIF and did not interfere with binding by therapeutic antibody h5D8 or M017C3.

### Example 25

### Determining LIF Levels in Patients

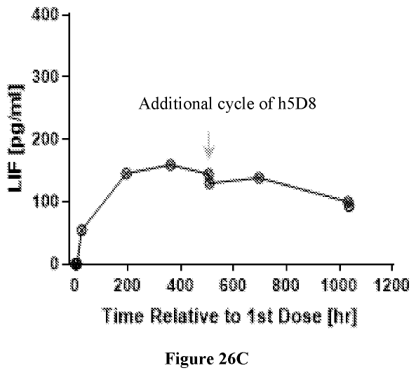

The LIF level assay as described in Example 23 was used to determine LIF levels in three human subjects with different cancers. Subject A is a 57-year-old white male diagnosed with a myxoid liposarcoma. Subject was treated with neoadjuvant Adriamycin and Ifosfamide for about 4 months and neoadjuvant radiotherapy (5000 cGy) to the right calf for about 35 days. Subject then underwent a curative wide resection of the right calf, dissection of the right posterior tibial nerve and popliteal, anterior and posterior tibial and peroneal vessels. Subject recurred with pleural disease and malignant lymphadenopathy in the chest. Subject was treated with Gemcitabine and Taxotere for about 41 days, the best response progressive disease. Subject was then treated with Dacarbazine for 4 months, with a best response of partial response. Subject had radiologic progression 22 days later and entered the h5D8 trial. Subject received his first dose of h5D8 1125 mg, and his most recent dose (C6) (1500 mg) about 136 days later. At baseline, Subject had no clinically significant laboratory abnormalities, and his ECOG performance status was 1. Subject has no peripheral tumor markers. Two pleural masses were selected as target lesions, and three non-target lesions were selected (1 pleural mass and 2 LNS). A biopsy was collected from a metastatic lung site to determine biomarkers for h5D8 treatment. At the time the biopsy was taken, Subject showed evidence of elevated LIF levels. LIF level of Subject A is shown in FIG. 26A.

Subject B is a 66-year-old male diagnosed with stage IV melanoma. Subject was administered one treatment prior to the h5D8 trial. The best response was not evaluable and the results are not shown (Nivolumab; 4 months). The failed Nivolumab treatment indicated profound tumor immune suppression. Subject is on the treatment regime (1125 mg) (6 weeks) and the best response recorded has been “progressive disease.” A biopsy was collected from a metastatic skin site. At the time the biopsy was collected, a generally higher level of LIF was observed in the Subject via the total LIF assay. The results of the total LIF assay are shown in FIG. 26B.

Subject C is a 66 year old white female diagnosed with ovarian cancer. Subject's h5D8 C5 assessment at 12 weeks showed an increase in CA19-9 (412 to 1072 U/ml) but her target lesions showed no significant increases by RECIST criteria (107 to 109 mm). Subject is on the treatment regime (1500 mg) (+16 weeks) and the best response recorded has been “stable disease.” A biopsy was collected from a metastatic lymph node site to determine biomarkers for h5D8 treatment. At the time the biopsy was taken, the Subject showed evidence of elevated LIF levels as a result of h5D8 administration. The LIF level of Subject C is shown in FIG. 26C.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, unless otherwise indicated, the term “about” refers to an amount that is near the stated amount by at least 10%.

As used herein the term “treat” or “treating” refers to interventions to a physiological or disease state of an individual designed or intended to ameliorate at least one sign or symptom associated with said physiological or disease state. Described herein treat or treating with respect to cancer refers to interventions intended to induce a complete response, a partial response, a delay of progression of the cancer or tumor being treated, a decrease in tumor size or tumor burden, or a delay in growth of tumor or tumor burden. Treating also refers to interventions intended to reduce metastases or malignancy of a cancer or a tumor. The skilled artisan will recognize that given a heterogeneous population of individuals afflicted with a disease, not all individuals will respond equally, or at all, to a given treatment. Nevertheless, these individuals are considered treated. Unsuccessful treatments generally result in progression of disease, and a necessity for additional treatment with a different therapeutic. In certain aspects the antibodies and methods described herein can be used to maintain remission of a cancer or prevent reoccurrence of the same cancer or a different cancer related to the treated cancer.

As used herein, the term “total LIF” refers to both LIF bound to a therapeutic antibody and LIF unbound to a therapeutic antibody. As used herein, the term “total LIF level” refers to a quantification of total LIF. As described herein, assays that measure total LIF level measure both LIF bound to a therapeutic antibody and LIF unbound to a therapeutic antibody. As described herein, total LIF level can be determined for a variety of patient samples, including but not limited to, blood, plasma, or serum.

SEQUENCES

| SEQ | |
|---|---|
| ID | |
| NO | Sequence |
| 1 | GFTFSHAWMH |
| 2 | GFTFSHAW |
| 3 | HAWMH |
| 4 | QIKAKSDDYATYYAESVKG |
| 5 | IKAKSDDYAT |
| 6 | TCWEWDLDF |
| 7 | WEWDLDF |
| 8 | TSWEWDLDF |
| 9 | RSSQSLLDSDGHTYLN |
| 10 | QSLLDSDGHTY |
| 11 | SVSNLES |
| 12 | SYS |
| 13 | MQATHAPPYT |
| 14 | EVQLVESGGGLVKPGGSLKLSCAASGFTFSHAWMHWVRQAPGKGLEWVAQIKAKSDDYATYYAESVKGRFTISR |
| | DDSKNTLYLQMNSLKTEDTAVYYCTCWEWDLDFWGQGTLVTVSS |
| 15 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTCWEWDLDFWGQGTMVTVSS |
| 16 | EVQLVESGGGVVQPGRSLRLSCAASGFTFSHAWMHWVRQAPGKGLEWVAQIKAKSDDYATYYAESVKGRFSISR |
| | DNAKNSLYLQMNSLRVEDTVVYYCTCWEWDLDFWGQGTTVTVSS |
| 17 | EVQLMESGGGLVKPGGSLRLSCATSGFTFSHAWMHWVRQAPGKGLEWVGQIKAKSDDYATYYAESVKGRFTISR |
| | DDSKSTLFLQMNNLKTEDTAVYYCTCWEWDLDFWGQGTLVTVSS |
| 18 | DVVMTQSPLSLPVTLGQPASISCRSSQSLLDSDGHTYLNWFQQRPGQSPRRLIYSVSNLESGVPDRFSGSGSGT |
| | DFTLKISRVEAEDVGLYYCMQATHAPPYTFGQGTKLEIK |
| 19 | DIVMTQTPLSSPVTLGQPASISCRSSQSLLDSDGHTYLNWLQQRPGQPPRLLIYSVSNLESGVPDRFSGSGAGT |
| | DFTLKISRVEAEDVGVYYCMQATHAPPYTFGQGTKLE1K |
| 20 | DIVMTQTPLSLSVTPGQPASISCRSSQSLLDSDGHTYLNWLLQKPGQPPQLLIYSVSNLESGVPNRFSGSGSGT |
| | DFTLKISRVEAEDVGLYYCMQATHAPPYTFGGGTKVEIK |
| 21 | DVVMTQSPLSQPVTLGQPASISCRSSQSLLDSDGHTYLNWLQQRPGQSPRRLIYSVSNLESGVPDRFNGSGSGT |
| | DFTLSISRVEAEDVGVYYCMQATHAPPYTFGQGTKVEIK |
| 22 | MGWTLVFLFLLSVTAGVHSEVQLVESGGGLVKPGGSLKLSCAASGFTFSHAWMHWVRQAPGKGLEWVAQIKAKS |
| | DDYATYYAESVKGRFTISRDDSKNTLYLQMNSLKTEDTAVYYCTCWEWDLDFWGQGTLVTVSSASTKGPSVFPL |
| | APSSKSTSGGTAALGCLVKDYFPEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKLTVDKSRWQQGNVFS |
| | CSVMHEALHNHYTQKSLSLSPGK |
| 23 | MGWTLVFLFLLSVTAGVHSQVQLQESGGGLVKPGGSLRLSCAASGFTFSHAWMHWVRQAPGKGLEWVGQIKAKS |
| | DDYATYYAESVKGRFTISRDDSKNTLYLQMNSLKTEDTAVYYCTCWEWDLDFWGQGTMVTVSSASTKGPSVFPL |
| | APSSKSTSGGTAALGCLVKDYFPEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKLTVDKSRWQQGNVFS |
| | CSVMHEALHNHYTQKSLSLSPGK |
| 24 | MGWTLVFLFLLSVTAGVHSEVQLVESGGGVVQPGRSLRLSCAASGFTFSHAWMHWVRQAPGKGLEWVAQIKAKS |
| | DDYATYYAESVKGRFSISRDNAKNSLYLQMNSLRVEDTVVYYCTCWEWDLDFWGQGTTVTVSSASTKGPSVFPL |
| | APSSKSTSGGTAALGCLVKDYFPEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKLTVDKSRWQQGNVFS |
| | CSVMHEALHNHYTQKSLSLSPGK |
| 25 | MGWTLVFLFLLSVTAGVHSEVQLMESGGGLVKPGGSLRLSCATSGFTFSHAWMHWVRQAPGKGLEWVGQIKAKS |
| | DDYATYYAESVKGRFTISRDDSKSTLFLQMNNLKTEDTAVYYCTCWEWDLDFWGQGTLVTVSSASTKGPSVFPL |
| | APSSKSTSGGTAALGCLVKDYFPEPVTVSWNSGALTSGVHTFPAVLQSSGLYSLSSVVTVPSSSLGTQTYICNV |
| | NHKPSNTKVDKKVEPKSCDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLDSDGSFFLYSKLTVDKSRWQQGNVFS |
| | CSVMHEALHNHYTQKSLSLSPGK |
| 26 | MVSSAQFLGLLLLCFQGTRCDVVMTQSPLSLPVTLGQPASISCRSSQSLLDSDGHTYLNWFQQRPGQSPRRLIY |
| | SVSNLESGVPDRFSGSGSGTDFTLKISRVEAEDVGLYYCMQATHAPPYTFGQGTKLEIKRTVAAPSVFIFPPSD |
| | EQLKSGTASVVCLLNNFYPREAKVQWKVDNALQSGNSQESVTEQDSKDSTYSLSSTLTLSKADYEKHKVYACEV |
| | THQGLSSPVTKSFNRGEC |
| 27 | MVSSAQFLGLLLLCFQGTRCDIVMTQTPLSSPVTLGQPASISCRSSQSLLDSDGHTYLNWLQQRPGQPPRLLIY |
| | SVSNLESGVPDRFSGSGAGTDFTLKISRVEAEDVGVYYCMQATHAPPYTFGQGTKLEIKRTVAAPSVFIFPPSD |
| | EQLKSGTASVVCLLNNFYPREAKVQWKVDNALQSGNSQESVTEQDSKDSTYSLSSTLTLSKADYEKHKVYACEV |
| | THQGLSSPVTKSFNRGEC |
| 28 | MVSSAQFLGLLLLCFQGTRCDIVMTQTPLSLSVTPGQPASISCRSSQSLLDSDGHTYLNWLLQKPGQPPQLLIY |
| | SVSNLESGVPNRFSGSGSGTDFTLKISRVEAEDVGLYYCMQATHAPPYTFGGGTKVEIKRTVAAPSVFIFPPSD |
| | EQLKSGTASVVCLLNNFYPREAKVQWKVDNALQSGNSQESVTEQDSKDSTYSLSSTLTLSKADYEKHKVYACEV |
| | THQGLSSPVTKSFNRGEC |
| 29 | MVSSAQFLGLLLLCFQGTRCDVVMTQSPLSQPVTLGQPASISCRSSQSLLDSDGHTYLNWLQQRPGQSPRRLIY |
| | SVSNLESGVPDRFSGSGSGTDFTLSISRVEAEDVGVYYCMQATHAPPYTFGQGTKVEIKRTVAAPSVFIFPPSD |
| | EQLKSGTASVVCLLNNFYPREAKVQWKVDNALQSGNSQESVTEQDSKDSTYSLSSTLTLSKADYEKHKVYACEV |
| | THQGLSSPVTKSFNRGEC |
| 30 | EVQLVESGGGLVKPGGSLKLSCAASGFTFSHAWMHWVRQAPGKGLEWVAQIKAKSDDYATYYAESVKGRFTISR |
| | DDSKNTLYLQMNSLKTEDTAVYYCTCWEWDLDFWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSRDELTKNQVSLTCLV |
| | KGFYPSDIAVEWESNGQPENNYKTTPPVLDSDGSFFLYSKLTVDKSRWQQGNVFSCSVMHEALHNHYTQKSLSL |
| | SPGK |
| 31 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTCWEWDLDFWGQGTMVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSRDELTKNQVSLTCLV |
| | KGFYPSDIAVEWESNGQPENNYKTTPPVLDSDGSFFLYSKLTVDKSRWQQGNVFSCSVMHEALHNHYTQKSLSL |
| | SPGK |
| 32 | EVQLVESGGGVVQPGRSLRLSCAASGFTFSHAWMHWVRQAPGKGLEWVAQIKAKSDDYATYYAESVKGRFSISR |
| | DNAKNSLYLQMNSLRVEDTVVYYCTCWEWDLDFWGQGTTVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSRDELTKNQVSLTCLV |
| | KGFYPSDIAVEWESNGQPENNYKTTPPVLDSDGSFFLYSKLTVDKSRWQQGNVFSCSVMHEALHNHYTQKSLSL |
| | SPGK |
| 33 | EVQLMESGGGLVKPGGSLRLSCATSGFTFSHAWMHWVRQAPGKGLEWVGQIKAKSDDYATYYAESVKGRFTISR |
| | DDSKSTLFLQMNNLKTEDTAVYYCTCWEWDLDFWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSRDELTKNQVSLTCLV |
| | KGFYPSDIAVEWESNGQPENNYKTTPPVLDSDGSFFLYSKLTVDKSRWQQGNVFSCSVMHEALHNHYTQKSLSL |
| | SPGK |
| 34 | DVVMTQSPLSLPVTLGQPASISCRSSQSLLDSDGHTYLNWFQQRPGQSPRRLIYSVSNLESGVPDRFSGSGSGT |
| | DFTLKISRVEAEDVGLYYCMQATHAPPYTFGQGTKLEIKRTVAAPSVFIFPPSDEQLKSGTASVVCLLNNFYPR |
| | EAKVQWKVDNALQSGNSQESVTEQDSKDSTYSLSSTLTLSKADYEKHKVYACEVTHQGLSSPVTKSFNRGEC |
| 35 | DIVMTQTPLSSPVTLGQPASISCRSSQSLLDSDGHTYLNWLQQRPGQPPRLLIYSVSNLESGVPDRFSGSGAGT |
| | DFTLKISRVEAEDVGVYYCMQATHAPPYTFGQGTKLEIKRTVAAPSVFIFPPSDEQLKSGTASVVCLLNNFYPR |
| | EAKVQWKVDNALQSGNSQESVTEQDSKDSTYSLSSTLTLSKADYEKHKVYACEVTHQGLSSPVTKSFNRGEC |
| 36 | DIVMTQTPLSLSVTPGQPASISCRSSQSLLDSDGHTYLNWLLQKPGQPPQLLIYSVSNLESGVPNRFSGSGSGT |
| | DFTLKISRVEAEDVGLYYCMQATHAPPYTFGGGTKVEIKRTVAAPSVFIFPPSDEQLKSGTASVVCLLNNFYPR |
| | EAKVQWKVDNALQSGNSQESVTEQDSKDSTYSLSSTLTLSKADYEKHKVYACEVTHQGLSSPVTKSFNRGEC |
| 37 | DVVMTQSPLSQPVTLGQPASISCRSSQSLLDSDGHTYLNWLQQRPGQSPRRLIYSVSNLESGVPDRFSGSGSGT |
| | DFTLSISRVEAEDVGVYYCMQATHAPPYTFGQGTKVEIKRTVAAPSVFIFPPSDEQLKSGTASVVCLLNNFYPR |
| | EAKVQWKVDNALQSGNSQESVTEQDSKDSTYSLSSTLTLSKADYEKHKVYACEVTHQGLSSPVTKSFNRGEC |
| 38 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTSWEWDLDFWGQGTMVTVSS |
| 39 | QVQLQESGGGLVKPGGSLRLSCAASGFTFSHAWMHWVRQAPGKGLEWVGQIKAKSDDYATYYAESVKGRFTISR |
| | DDSKNTLYLQMNSLKTEDTAVYYCTSWEWDLDFWGQGTMVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSREEMTKNQVSLTCLV |
| | KGFYPSDIAVEWESNGQPENNYKTTPPVLDSDGSFFLYSKLTVDKSRWQQGNVFSCSVMHEALHNHYTQKSLSL |
| | SPGK |
| 40 | SPLPITPVNATCAIRHPCHNNLMNQIRSQLAQLNGSANALFILYYTAQGEPFPNNLDKLCGPNVTDFPPFHANG |
| | TEKAKLVELYRIVVYLGTSLGNITRDQKILNPSALSLHSKLNATADILRGLLSNVLCRLCSKYHVGHVDVTYGP |
| | DTSGKDVFQKKKLGCQLLGKYKQIIAVLAQAF |
| 41 | EQLEESVGDLVKPGASLTLTCTASGFSFSGLYYMCWVRQAPGKGLEWIACIWTGSTDSTYYATWAKGRFTISKT |
| | SSTTVTLQMTSLTVADTATYFCARGGGVPGDGYALWGPGTLVTVSS |
| 42 | GTELVMTQTPASVSEPVGGTVTINCQASEDISSNLVWYQQKSGQPPKLLIYDASMLASGVPSRFKGSGSGTQFT |
| | LTISDLECADGATYYCQSYYVASSSYFVNGFGGGTEVV |
| 43 | QSLEESGGDLVKPEGSLTLTCTASGFSFSTDYWICWVRQAPGKGLEWIACIYVGTSGDTYYATWAKGRFTISKT |
| | SSTTVTLQMTSLTAADTATYFCAGADNPYDYFNLWGPGTLVTVSS |
| 44 | GTPDMTQTPASMEVAVGGTVTIKCQASETISGYLSWYQQKPGQRPKLLMYRASTLASGVSSRFKGSGSGTQFTL |
| | TISGVECADAATYYCQQGYSYSDTDNVFGGGTEVV |
| 45 | EQLVESGGGLVQPEGSLTLTCKASGIDFSSNYWICWVRQAPGKGLEWIACIYVGSSGDTYYANWAKGRFAISKT |
| | SSTTVTLEVTSLTAADTATYFCARTVEPYDNLHFWGPGTLVSVSS |
| 46 | GTPEMTQTPASMEVAVGGTVTIKCQASETISGYLSWYQQKPGQRPKLLMYRASTLASGVSSRFKGSGSGTQFTL |
| | TISGVECADAATYYCQQGYSYSDTDNVFGGGTEVV |

What is claimed is:

1. A Leukemia Inhibitory Factor (LIF) complex, the complex comprising: LIF, a LIF capture antibody that specifically binds to LIF, a LIF detecting antibody that specifically binds to LIF, and optionally a LIF therapeutic antibody that specifically binds LIF, wherein the LIF detecting or the LIF capture antibody comprises A4 or a LIF binding fragment thereof .

2. Use of the LIF complex of claim 1, wherein the LIF capture antibody or the LIF detecting antibody does not compete for binding with the LIF therapeutic antibody.

3. The LIF complex of any one of claims 1 to 2, wherein the LIF therapeutic antibody comprises:
a) an immunoglobulin heavy chain complementarity determining region 1 (VH-CDR1) comprising the amino acid sequence set forth in any one of SEQ ID NOs: 1-3;
b) an immunoglobulin heavy chain complementarity determining region 2 (VH-CDR2) comprising the amino acid sequence set forth in any one of SEQ ID NOs: 4 or 5;
c) an immunoglobulin heavy chain complementarity determining region 3 (VH-CDR3) comprising the amino acid sequence set forth in any one of SEQ ID NOs: 6-8;
d) an immunoglobulin light chain complementarity determining region 1 (VL-CDR1) comprising the amino acid sequence set forth in any one of SEQ ID NOs: 9 or 10;
e) an immunoglobulin light chain complementarity determining region 2 (VL-CDR2) comprising the amino acid sequence set forth in any one of SEQ ID NOs: 11 or 12; and
f) an immunoglobulin light chain complementarity determining region 3 (VL-CDR3) comprising the amino acid sequence set forth in SEQ ID NO: 13.

4. The LIF complex of any one of claims 1 to 2, wherein the LIF therapeutic antibody comprises:
a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and
n immunoglobulin light chain variable region (VL) sequence with an amino acid sequence at least about 80%, 90%, 95%, 97%, 98%, or 99% identical to the amino acid sequence set forth in any one of SEQ ID NOs: 18-21.

5. The LIF complex of any one of claims 1 to 2, wherein the LIF therapeutic antibody comprises:
a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and
b) an immunoglobulin light chain sequence with an amino acid sequence at least about 80%, 90%, 95%, 97%, 98%, or 99% identical to the amino acid sequence set forth in any one of SEQ ID NOs: 34-37.

6. The LIF complex of any one of claims 1 to 5, wherein the LIF capture antibody is coupled to a surface, wherein the surface comprises an electrically conductive substance, or wherein the electrically conductive substance is an electrode.

7. The LIF complex of any one of claims 1 to 5, wherein the LIF detecting antibody is coupled to a detectable moiety, wherein the detectable moiety that generates a chemical signal, an electrochemical signal, a luminescent signal, or a fluorescent signal, or wherein the detectable moiety generates an electrochemical signal

8. The LIF complex of any one of claims 1 to 7, wherein the LIF detecting antibody and the LIF capture antibody do not bind to a region of LIF that physically interacts with gp130.

9. The LIF complex of any one of claims 1 to 8, wherein the LIF capture antibody of the LIF detecting antibody comprises:
a) an immunoglobulin heavy chain variable region sequence with an amino acid sequence at least about 80%, 90%, 95%, 97%, 98%, or 99% identical to the amino acid sequence set forth in SEQ ID NO: 41; and
b) an immunoglobulin light chain variable region sequence with an amino acid sequence at least about 80%, 90%, 95%, 97%, 98%, or 99% identical to the amino acid sequence set forth in SEQ ID NO: 42.

10. The LIF complex of any one of claims 1 to 9, wherein the complex is contained in at least one well of a multi-well plate, wherein the complex is contained in at least one well of a 96-well plate, a 384-well plate, or a 1536-well plate, or wherein the complex is detectable at a level of 1 nanogram per milliliter.

11. A method of quantifying Leukemia Inhibitory Factor (LIF) in a sample from an individual comprising LIF comprising:
a) contacting the sample comprising LIF to a capture antibody that specifically binds to LIF;
b) contacting the sample comprising LIF to a detecting antibody that specifically binds LIF;
c) detecting the LIF in the sample that is bound to the capture antibody and the detecting antibody;
wherein the LIF detecting or the LIF capture antibody comprises A4 or a LIF binding fragment thereof .

12. The method of claim 11, wherein the individual has been treated with a LIF therapeutic antibody.

13. The method of any one of claims 11 to 12, wherein the LIF therapeutic antibody comprises:
a) an immunoglobulin heavy chain complementarity determining region 1 (VH-CDR1) comprising the amino acid sequence set forth in any one of SEQ ID NOs: 1-3;
b) an immunoglobulin heavy chain complementarity determining region 2 (VH-CDR2) comprising the amino acid sequence set forth in any one of SEQ ID NOs: 4 or 5;
c) an immunoglobulin heavy chain complementarity determining region 3 (VH-CDR3) comprising the amino acid sequence set forth in any one of SEQ ID NOs: 6-8;
d) an immunoglobulin light chain complementarity determining region 1 (VL-CDR1) comprising the amino acid sequence set forth in any one of SEQ ID NOs: 9 or 10;
e) an immunoglobulin light chain complementarity determining region 2 (VL-CDR2) comprising the amino acid sequence set forth in any one of SEQ ID NOs: 11 or 12; and
f) an immunoglobulin light chain complementarity determining region 3 (VL-CDR3) comprising the amino acid sequence set forth in SEQ ID NO: 13.

14. The method of any one of claims 11 to 12, wherein the LIF therapeutic antibody comprises:
a) an immunoglobulin heavy chain variable region (VH) sequence with an amino acid sequence at least about 80%, 90%, 95%, 97%, 98%, or 99% identical to the amino acid sequence set forth in any one of SEQ ID NOs: 14, 15, 17 or 38; and
n immunoglobulin light chain variable region (VL) sequence with an amino acid sequence at least about 80%, 90%, 95%, 97%, 98%, or 99% identical to the amino acid sequence set forth in any one of SEQ ID NOs: 18-21.

15. The method of any one of claims 11 to 12, wherein the LIF therapeutic antibody comprises:
a) an immunoglobulin heavy chain sequence with an amino acid sequence at least about 80%, 90%, 95%, 97%, 98%, or 99% identical to the amino acid sequence set forth in any one of SEQ ID NOs: 30-33 or 39; and
b) an immunoglobulin light chain sequence with an amino acid sequence at least about 80%, 90%, 95%, 97%, 98%, or 99% identical to the amino acid sequence set forth in any one of SEQ ID NOs: 34-37.

16. The method of any one of claims 11 to 15, wherein the LIF capture antibody is coupled to a surface, wherein the surface comprises an electrically conductive substance, or wherein the electrically conductive substance is an electrode.

17. The method of any one of claims 11 to 15, wherein the LIF detecting antibody is coupled to a detectable moiety, wherein the detectable moiety that generates a chemical signal, an electrochemical signal, a luminescent signal, or a fluorescent signal, or wherein the detectable moiety that generates an electrochemical signal.

18. The method of any one of claims 11 to 17, wherein the LIF capture antibody and the LIF detecting antibody do not bind to a region of LIF that physically interacts with gp130.

19. The method of any one of claims 11 to 18, wherein the LIF capture antibody or the LIF detecting antibody comprises:
a) an immunoglobulin heavy chain variable region sequence with an amino acid sequence at least about 80%, 90%, 95%, 97%, 98%, or 99% identical to the amino acid sequence set forth in SEQ ID NO: 41; and
b) an immunoglobulin light chain variable region sequence with an amino acid sequence at least about 80%, 90%, 95%, 97%, 98%, or 99% identical to the amino acid sequence set forth in SEQ ID NO: 42.

20. The method of any one of claims 11 to 19, wherein the sample comprising LIF is contained in at least one well of a multi-well plate, or wherein the sample comprising LIF is contained in at least one well of a 96-well plate, a 384-well plate, or a 1536-well plate.

21. A Leukemia Inhibitory Factor (LIF) binding antibody or fragment thereof, wherein the LIF binding antibody or fragment thereof comprises:
a) an immunoglobulin heavy chain variable region with an amino acid sequence at least about 90% identical to the amino acid sequence set forth in SEQ ID NO: 41; and
b) an immunoglobulin light chain variable region with an amino acid sequence at least about 90% identical to the amino acid sequence set forth in SEQ ID NO: 42.

22. The LIF binding antibody or fragment thereof of claim 21, wherein immunoglobulin heavy chain variable region comprises an amino acid sequence at least about 95% identical to the amino acid sequence set forth in SEQ ID NO: 41; and
a) the immunoglobulin light chain variable region comprises an amino acid sequence at least about 95% identical to the amino acid sequence set forth in SEQ ID NO: 42.

23. The LIF binding antibody or fragment thereof of any one of claim 21 or 22, wherein the immunoglobulin heavy chain variable region comprises the amino acid sequence set forth in SEQ ID NO: 41; and
a) the immunoglobulin light chain variable region comprises the amino acid sequence set forth in SEQ ID NO: 42.

24. The LIF binding antibody or fragment thereof of any one of claims 21 to 23, wherein the LIF binding antibody is coupled to a detectable moiety, wherein the detectable moiety generates a chemical signal, an electrochemical signal, a luminescent signal, or a fluorescent signal, or wherein the detectable moiety generates an electrochemical signal.

25. The LIF binding antibody or fragment thereof of any one of claims 21 to 23, wherein the LIF binding antibody is specifically bound to LIF.


SEQUENCE LISTING (46 sequences)

SEQ ID NO: 1 - protein, 10 aa, Artificial Sequence
  Synthetic Peptide
GFTFSHAWMH

SEQ ID NO: 2 - protein, 8 aa, Artificial Sequence
  Synthetic Protein
GFTFSHAW

SEQ ID NO: 3 - protein, 5 aa, Artificial Sequence
  Synthetic Protein
HAWMH

SEQ ID NO: 4 - protein, 19 aa, Artificial Sequence
  Synthetic Protein
QIKAKSDDYATYYAESVKG

SEQ ID NO: 5 - protein, 10 aa, Artificial Sequence
  Synthetic Protein
IKAKSDDYAT

SEQ ID NO: 6 - protein, 9 aa, Artificial Sequence
  Synthetic Protein
TCWEWDLDF

SEQ ID NO: 7 - protein, 7 aa, Artificial Sequence
  Synthetic Protein
WEWDLDF

SEQ ID NO: 8 - protein, 9 aa, Artificial Sequence
  Synthetic Protein
TSWEWDLDF

SEQ ID NO: 9 - protein, 16 aa, Artificial Sequence
  Synthetic Protein
RSSQSLLDSDGHTYLN

SEQ ID NO: 10 - protein, 11 aa, Artificial Sequence
  Synthetic Protein
QSLLDSDGHTY

SEQ ID NO: 11 - protein, 7 aa, Artificial Sequence
  Synthetic Protein
SVSNLES

SEQ ID NO: 12 - protein, 3 aa, Artificial Sequence
  Synthetic Protein
SVS

SEQ ID NO: 13 - protein, 10 aa, Artificial Sequence
  Synthetic Protein
MQATHAPPYT

SEQ ID NO: 14 - protein, 118 aa, Artificial Sequence
  Synthetic Protein
EVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSS

SEQ ID NO: 15 - protein, 118 aa, Artificial Sequence
  Synthetic Protein
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSS

SEQ ID NO: 16 - protein, 118 aa, Artificial Sequence
  Synthetic Protein
EVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSS

SEQ ID NO: 17 - protein, 118 aa, Artificial Sequence
  Synthetic Protein
EVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSS

SEQ ID NO: 18 - protein, 113 aa, Artificial Sequence
  Synthetic Protein
DVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIK

SEQ ID NO: 19 - protein, 113 aa, Artificial Sequence
  Synthetic Protein
DIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIK

SEQ ID NO: 20 - protein, 113 aa, Artificial Sequence
  Synthetic Protein
DIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIK

SEQ ID NO: 21 - protein, 113 aa, Artificial Sequence
  Synthetic Protein
DVVMTQSPLSQPVTLGQPASISCRSSQSLLDSDGHTYLNWLQQRPGQSPRRLIYSVSNLESGVPDRFNGSGSGTDFTLSISRVEAEDVGVYYCMQATHAPPYTFGQGTKVEIK

SEQ ID NO: 22 - protein, 467 aa, Artificial Sequence
  Synthetic Protein
MGWTLVFLFLLSVTAGVHSEVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 467 aa, Artificial Sequence
  Synthetic Protein
MGWTLVFLFLLSVTAGVHSQVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 467 aa, Artificial Sequence
  Synthetic Protein
MGWTLVFLFLLSVTAGVHSEVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 467 aa, Artificial Sequence
  Synthetic Protein
MGWTLVFLFLLSVTAGVHSEVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 240 aa, Artificial Sequence
  Synthetic Protein
MVSSAQFLGLLLLCFQGTRCDVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 240 aa, Artificial Sequence
  Synthetic Protein
MVSSAQFLGLLLLCFQGTRCDIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 240 aa, Artificial Sequence
  Synthetic Protein
MVSSAQFLGLLLLCFQGTRCDIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 240 aa, Artificial Sequence
  Synthetic Protein
MVSSAQFLGLLLLCFQGTRCDVVMTQSPLSQPVTLGQPASISCRSSQSLLDSDGHTYLNWLQQRPGQSPRRLIYSVSNLESGVPDRFSGSGSGTDFTLSISRVEAEDVGVYYCMQATHAPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 448 aa, Artificial Sequence
  Synthetic Protein
EVQLVESGGGLVKPGGSLKLSCAASGFTFSHAWMHWVRQAPGKGLEWVAQIKAKSDDYATYYAESVKGRFTISRDDSKNTLYLQMNSLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 448 aa, Artificial Sequence
  Synthetic Protein
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTCWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 448 aa, Artificial Sequence
  Synthetic Protein
EVQLVESGGGVVQPGRSLRLSCAASGFTFSHAWMHWVRQAPGKGLEWVAQIKAKSDDYATYYAESVKGRFSISRDNAKNSLYLQMNSLRVEDTVVYYCTCWEWDLDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 448 aa, Artificial Sequence
  Synthetic Protein
EVQLMESGGGLVKPGGSLRLSCATSGFTFSHAWMHWVRQAPGKGLEWVGQIKAKSDDYATYYAESVKGRFTISRDDSKSTLFLQMNNLKTEDTAVYYCTCWEWDLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 220 aa, Artificial Sequence
  Synthetic Protein
DVVMTQSPLSLPVTLGQPASISCRSSQSLLDSDGHTYLNWFQQRPGQSPRRLIYSVSNLESGVPDRFSGSGSGTDFTLKISRVEAEDVGLYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 220 aa, Artificial Sequence
  Synthetic Protein
DIVMTQTPLSSPVTLGQPASISCRSSQSLLDSDGHTYLNWLQQRPGQPPRLLIYSVSNLESGVPDRFSGSGAGTDFTLKISRVEAEDVGVYYCMQATHAPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 220 aa, Artificial Sequence
  Synthetic Protein
DIVMTQTPLSLSVTPGQPASISCRSSQSLLDSDGHTYLNWLLQKPGQPPQLLIYSVSNLESGVPNRFSGSGSGTDFTLKISRVEAEDVGLYYCMQATHAPPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 220 aa, Artificial Sequence
  Synthetic Protein
DVVMTQSPLSQPVTLGQPASISCRSSQSLLDSDGHTYLNWLQQRPGQSPRRLIYSVSNLESGVPDRFSGSGSGTDFTLSISRVEAEDVGVYYCMQATHAPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 118 aa, Artificial Sequence
  Synthetic Protein
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTSWEWDLDFWGQGTMVTVSS

SEQ ID NO: 39 - protein, 448 aa, Artificial Sequence
  Synthetic Protein
QVQLQESGGGLVKPGGSLRLSCAASGFTFSHAWMHWVRQAPGKGLEWVGQIKAKSDDYATYYAESVKGRFTISRDDSKNTLYLQMNSLKTEDTAVYYCTSWEWDLDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 180 aa, Artificial Sequence
  Synthetic Protein
SPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 41 - protein, 120 aa, Artificial Sequence
  Synthetic Protein
EQLEESVGDLVKPGASLTLTCTASGFSFSGLYYMCWVRQAPGKGLEWIACIWTGSTDSTYYATWAKGRFTISKTSSTTVTLQMTSLTVADTATYFCARGGGVPGDGYALWGPGTLVTVSS

SEQ ID NO: 42 - protein, 112 aa, Artificial Sequence
  Synthetic Protein
GTELVMTQTPASVSEPVGGTVTINCQASEDISSNLVWYQQKSGQPPKLLIYDASMLASGVPSRFKGSGSGTQFTLTISDLECADGATYYCQSYYVASSSYFVNGFGGGTEVV

SEQ ID NO: 43 - protein, 119 aa, Artificial Sequence
  Synthetic Protein
QSLEESGGDLVKPEGSLTLTCTASGFSFSTDYWICWVRQAPGKGLEWIACIYVGTSGDTYYATWAKGRFTISKTSSTTVTLQMTSLTAADTATYFCAGADNPYDYFNLWGPGTLVTVSS

SEQ ID NO: 44 - protein, 109 aa, Artificial Sequence
  Synthetic Protein
GTPDMTQTPASMEVAVGGTVTIKCQASETISGYLSWYQQKPGQRPKLLMYRASTLASGVSSRFKGSGSGTQFTLTISGVECADAATYYCQQGYSYSDTDNVFGGGTEVV

SEQ ID NO: 45 - protein, 119 aa, Artificial Sequence
  Synthetic Protein
EQLVESGGGLVQPEGSLTLTCKASGIDFSSNYWICWVRQAPGKGLEWIACIYVGSSGDTYYANWAKGRFAISKTSSTTVTLEVTSLTAADTATYFCARTVEPYDNLHFWGPGTLVSVSS

SEQ ID NO: 46 - protein, 109 aa, Artificial Sequence
  Synthetic Peptide
GTPEMTQTPASMEVAVGGTVTIKCQASETISGYLSWYQQKPGQRPKLLMYRASTLASGVSSRFKGSGSGTQFTLTISGVECADAATYYCQQGYSYSDTDNVFGGGTEVV